(19) United States
(12) Patent Application Publication
(10) Pub. No.: US 2022/0411367 A1
(43) Pub. Date: Dec. 29, 2022

(54) ARYLMETHYLENE HETEROCYCLIC COMPOUNDS AS KV1.3 POTASSIUM SHAKER CHANNEL BLOCKERS

(71) Applicant: D. E. Shaw Research, LLC, New York, NY (US)

(72) Inventors: Fabrizio GIORDANETTO, New York, NY (US); Morten Østergaard JENSEN, Copenhagen (DK); Vishwanath JOGINI, Hyderabad (IN); Roger John SNOW, Danbury, CT (US)

(21) Appl. No.: 17/766,889

(22) PCT Filed: Oct. 6, 2020
(86) PCT No.: PCT/US2020/054360

§ 371 Date: Apr. 6, 2022

Related U.S. Application Data
(60) Provisional application No. 62/911,652, filed on Oct. 7, 2019.

(51) Int. Cl. C07D 207/06; C07D 471/04; C07D 491/107; C07D 223/04; C07D 205/04; C07D 491/113; C07D 401/04; C07D 401/06; C07D 413/06; C07D 471/10; C07D 405/04; C07D 487/04

(52) U.S. Cl. CPC C07D 207/06; C07D 471/04; C07D 491/107; C07D 223/04; C07D 205/04; C07D 491/113; C07D 401/04; C07D 401/06; C07D 413/06; C07D 471/10; C07D 405/04; C07D 487/04

(57) ABSTRACT

A compound of Formula (I), or a pharmaceutically acceptable salt thereof, is described, wherein the substituents are as defined herein. Pharmaceutical compositions comprising the same and method of using the same are also described.

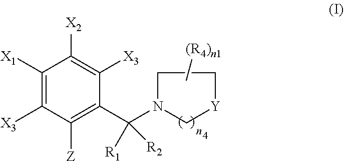

This application claims the benefit of and priority to U.S. Provisional Patent Application No. 62/911,652, filed on Oct. 7, 2019, the content of which is hereby incorporated by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals as potassium channel blockers.

### BACKGROUND

Voltage-gated Kv1.3 potassium (K⁺) channels are expressed in lymphocytes (T and B lymphocytes), the central nervous system, and other tissues and regulate a large number of physiological processes such as neurotransmitter release, heart rate, insulin secretion, and neuronal excitability. Kv1.3 channels can regulate membrane potential and thereby indirectly influence calcium signaling in human effector memory T cells. Effector memory T cells are mediators of several conditions, including multiple sclerosis, Type I diabetes mellitus, psoriasis, spondylitis, parodontitis, and rheumatoid arthritis. Upon activation, effector-memory T cells increase expression of the Kv1.3 channel. Amongst human B cells, naive and early memory B cells express small numbers of Kv1.3 channels when they are quiescent. In contrast, class-switched memory B cells express high numbers of Kv1.3 channels. Furthermore, the Kv1.3 channel promotes the calcium homeostasis required for T-cell receptor-mediated cell activation, gene transcription, and proliferation (Panyi, G., et al., 2004, Trends Immunol., 565-569). Blockade of Kv1.3 channels in effector memory T cells suppresses activities like calcium signaling, cytokine production (interferon-gamma, interleukin 2) and cell proliferation.

Autoimmune Disease is a family of disorders resulting from tissue damage caused by attack from the body's own immune system. Such diseases may affect a single organ, as in multiple sclerosis and Type I diabetes mellitus, or may involve multiple organs as in the case of rheumatoid arthritis and systemic lupus erythematosus. Treatment is generally palliative, with anti-inflammatory and immunosuppressive drugs, which can have severe side effects. A need for more effective therapies has led to search for drugs that can selectively inhibit the function of effector memory T cells, known to be involved in the etiology of autoimmune diseases. These inhibitors are thought to be able to ameliorate autoimmune diseases symptoms without compromising the protective immune response. Effector memory T cells (TEMs) express high numbers of the Kv1.3 channel and depend on these channels for their function. In vivo, Kv1.3 channel blockers paralyze TEMs at the sites of inflammation and prevent their reactivation in inflamed tissues. Kv1.3 channel blockers do not affect the motility within lymph nodes of naive and central memory T cells. Suppressing the function of these cells by selectively blocking the Kv1.3 channel offers the potential for effective therapy of autoimmune diseases with minimal side effects.

Multiple Sclerosis (MS) is caused by autoimmune damage to the Central Nervous System (CNS). Symptoms include muscle weakness and paralysis, which severely affect quality of life for patients. MS progresses rapidly and unpredictably and eventually leads to death. The Kv1.3 channel is also highly expressed in auto-reactive effector memory T cells from MS patients (Wulff H., et al., 2003, J. Clin. Invest., 1703-1713; Rus H., et al., 2005, PNAS, 11094-11099). Animal models of multiple sclerosis have been successfully treated using blockers of the Kv1.3 channel.

Compounds which are selective Kv1.3 channel blockers are thus potential therapeutic agents as immunosuppressants or immune system modulators. The Kv1.3 channel is also considered as a therapeutic target for the treatment of obesity and for enhancing peripheral insulin sensitivity in patients with type-2 diabetes mellitus. These compounds can also be utilized in the prevention of graft rejection, and the treatment of immunological (e.g., autoimmune) and inflammatory disorders.

Tubulointerstitial fibrosis is a progressive connective tissue deposition on the kidney parenchyma, leading to renal function deterioration and is involved in the pathology of chronic kidney disease, chronic renal failure, nephritis, and inflammation in glomeruli and is a common cause of end-stage renal failure. Overexpression of Kv1.3 channels in lymphocytes can promote their proliferation leading to chronic inflammation and overstimulation of cellular immunity, which are involved in the underlying pathology of these renal diseases and are contributing factors in the progression of tubulointerstitial fibrosis. Inhibition of the lymphocyte Kv1.3 channel currents suppress proliferation of kidney lymphocytes and ameliorate the progression of renal fibrosis (Kazama I., et al., 2015, Mediators Inflamm., 1-12).

Kv1.3 channels also play a role in gastroenterological disorders including inflammatory bowel diseases (IBD) such as ulcerative colitis (UC) and Crohn's disease. Ulcerative colitis is a chronic IBD characterized by excessive T-cell infiltration and cytokine production. Ulcerative colitis can impair quality of life and can lead to life-threatening complications. High levels of Kv1.3 channels in CD4 and CD8 positive T-cells in the inflamed mucosa of UC patients have been associated with production of pro-inflammatory compounds in active UC. Kv1.3 channels are thought to serve as a marker of disease activity and pharmacological blockade might constitute a novel immunosuppressive strategy in UC. Present treatment regimens for UC, including corticosteroids, salicylates, and anti-TNF-α reagents, are insufficient for many patients (Hansen L. K., et al., 2014, J. Crohns Colitis, 1378-1391). Crohn's disease is a type of IBD which may affect any part of the gastrointestinal tract. Crohn's disease is thought to be the result of intestinal inflammation due to a T-cell-driven process initiated by normally safe bacteria. Thus, Kv1.3 channel inhibition can be utilized in treating the Crohn's disease.

In addition to T cells, Kv1.3 channels are also expressed in microglia, where the channel is involved in inflammatory cytokine and nitric oxide production and in microglia-mediated neuronal killing. In humans, strong Kv1.3 channel expression has been found in microglia in the frontal cortex of patients with Alzheimer's disease and on CD68⁺ cells in multiple sclerosis brain lesions. It has been suggested that Kv1.3 channel blockers might be able to preferentially target detrimental proinflammatory microglia functions. Kv1.3 channels are expressed on activated microglia in infarcted rodent and human brain. Higher Kv1.3 channel current densities are observed in acutely isolated microglia from the infarcted hemisphere than in microglia isolated from the contralateral hemisphere of a mouse model of stroke (Chen Y. J., et al., 2017, Ann. Clin. Transl. Neurol., 147-161).

Expression of Kv1.3 channels is elevated in microglia of human Alzheimer's disease brains, suggesting that Kv1.3 channel is a pathologically relevant microglial target in Alzheimer's disease (Rangaraju S., et al., 2015, J. Alzheimers Dis., 797-808). Soluble AβO enhances microglial Kv1.3 channel activity. Kv1.3 channels are required for AβO-induced microglial pro-inflammatory activation and neurotoxicity. Kv1.3 channel expression/activity is upregulated in transgenic Alzheimer's disease animals and human Alzheimer's disease brains. Pharmacological targeting of microglial Kv1.3 channels can affect hippocampal synaptic plasticity and reduce amyloid deposition in APP/PS1 mice. Thus, Kv1.3 channel may be a therapeutic target for Alzheimer's disease.

Kv1.3 channel blockers could be also useful for ameliorating pathology in cardiovascular disorders such as ischemic stroke, where activated microglia significantly contributes to the secondary expansion of the infarct.

Kv1.3 channel expression is associated with the control of proliferation in multiple cell types, apoptosis, and cell survival. These processes are crucial for cancer progression. In this context, Kv1.3 channels located in the inner mitochondrial membrane can interact with the apoptosis regulator Bax (Serrano-Albarras, A., et al., 2018, Expert Opin. Ther. Targets, 101-105). Thus, inhibitors of Kv1.3 channels may be used as anticancer agents.

A number of peptide toxins with multiple disulfide bonds from spiders, scorpions, and anemones are known to block Kv1.3 channels. A few selective, potent peptide inhibitors of the Kv1.3 channel have been developed. A synthetic derivative of stichodactyla toxin (shk) with an unnatural amino acid (shk-186) is the most advanced peptide toxin. Shk has demonstrated efficacy in preclinical models and is currently in a phase I clinical trial for treatment of psoriasis. Shk can suppress proliferation of TEM cells resulting in improved condition in animal models of multiple sclerosis. Unfortunately, Shk also binds to the closely-related Kvi channel subtype found in CNS and the heart. There is a need for Kv1.3 channel-selective inhibitors to avoid potential cardio- and neuro-toxicity. Additionally, small peptides like shk-186 are rapidly cleared from the body after administration, resulting in short circulating half-lives, frequent administration events. Thus, there is a need for the development of long-acting, selective Kv1.3 channel inhibitors for the treatment of chronic inflammatory diseases.

Thus, there remains a need for development of novel Kv1.3 channel blockers as pharmaceutical agents.

### SUMMARY OF THE INVENTION

In one aspect, compounds useful as potassium channel blockers having a structure of Formula I

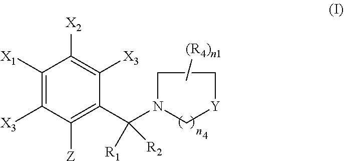

are described, where the various substituents are defined herein. The compounds of Formula I described herein can block Kv1.3 potassium (K⁺) channels and be used in the treatment of a variety of conditions. Methods for synthesizing these compounds are also described herein. Pharmaceutical compositions and methods of using these compositions described herein are useful for treating conditions in vitro and in vivo. Such compounds, pharmaceutical compositions, and methods of treatment have a number of clinical applications, including as pharmaceutically active agents and methods for treating cancer, an immunological disorder, a Central Nerve System (CNS) disorder, an inflammatory disorder, a gastroenterological disorder, a metabolic disorder, a cardiovascular disorder, a kidney disease or a combination thereof.

In one aspect, a compound of Formula I or a pharmaceutically acceptable salt thereof is described,

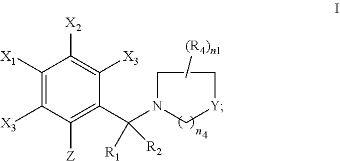

where

each occurrence of Y is independently C(R₄)₂, NR₄, O, S, SO, SO₂, or SO(═NRₐ);

Z is ORₐ;

X₁ is H, halogen, CN, alkyl, halogenated alkyl, cycloalkyl, or halogenated cycloalkyl;

X₂ is H, halogen, CN, alkyl, halogenated alkyl, cycloalkyl, or halogenated cycloalkyl;

each occurrence of X₃ is independently H, halogen, CN, alkyl, halogenated alkyl, cycloalkyl, or halogenated cycloalkyl;

R₁ and R₂ are each independently H, alkyl, (CR₆R₇)ₙ₃ORₐ, (CR₆R₇)ₙ₃NRₐR_{b}, (CR₆R₇)ₙ₃(C═O)NR_{b}Rₐ, or (CR₆R₇)ₙ₃NR_{b}(C═O)Rₐ;

each occurrence of R₄ is independently H, halogen, alkyl, cycloalkyl, halogenated alkyl, halogenated cycloalkyl, optionally substituted saturated heterocycle, optionally substituted aryl, optionally substituted heteroaryl, CN, oxo, (C═O)R_{b}, (C═O)OR_{b}, (CR₆R₇)ₙ₃ORₐ, (CR₆R₇)ₙ₃NRₐR_{b}, (CR₆R₇)ₙ₃SO₂Rₐ, (CR₆R₇)ₙ₃SO₂NRₐR_{b}, (CR₆R₇)ₙ₃NRₐSO₂R_{b}, (CR₆R₇)ₙ₃NRₐ(C═O)R_{b}, (CR₆R₇)ₙ₃(C═O)NRₐR_{b}, (CR₆R₇)ₙ₃NRₐ(C═O)NRₐR_{b}, (C═O)(CR₆R₇)ₙ₃OR_{b}, (C═O)(CR₆R₇)ₙ₃NRₐR_{b}, or an optionally substituted 5- or 6-membered heterocycle containing 1-3 heteroatoms each selected from the group consisting of N, O, and S;

or two R₄ taken together forming an optionally substituted carbocycle, saturated heterocycle, or heteroaryl containing 0-3 heteroatoms each selected from the group consisting of N, O, and S;

each occurrence of R₆ and R₇ are independently H, alkyl, cycloalkyl, optionally substituted aryl, or optionally substituted heteroaryl;

each occurrence of Rₐ and R_{b} are independently H, alkyl, alkenyl, cycloalkyl, halogenated alkyl, halogenated cycloalkyl, optionally substituted saturated heterocycle, optionally substituted aryl, or optionally substituted heteroaryl; or alternatively Rₐ and R_{b} together with the nitrogen atom that they are connected to form an optionally substituted heterocycle including the nitrogen atom and 0-3 additional heteroatoms each selected from the group consisting of N, O, and S;

the alkyl, cycloalkyl, carbocycle, heterocycle, aryl, and heteroaryl in X₁, X₂, X₃, R₁, R₂, R₄, R₆, and R₇, where applicable, are optionally substituted by 1-4 substituents each independently selected from the group consisting of alkyl, cycloalkyl, halogenated cycloalkyl, halogenated alkyl, halogen, (CRₐR_{b})ₙ₃ORₐ, (CRₐR_{b})ₙ₃NRₐR_{b}, (CRₐR_{b})ₙ₃NRₐ(C═O)R_{b}, (CRₐR_{b})ₙ₃(C═O)NRₐR_{b}, and oxo where valence permits;

each occurrence of n₁ is independently an integer from 0-4 where valence permits;

each occurrence of n₃ is independently an integer from 0-4; and

each occurrence of n₄ is independently 0, 1, or 2.

In any one of the embodiments described herein, the structural moiety

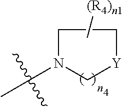

has the structure of

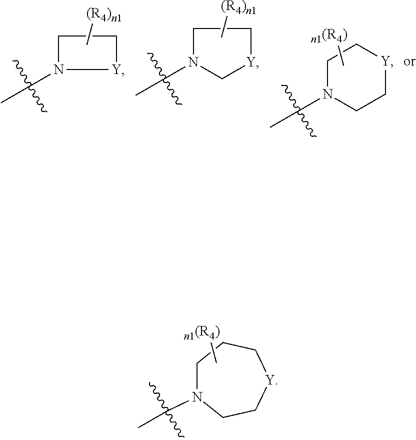

In any one of the embodiments described herein, Y is C(R₄)₂.

In any one of the embodiments described herein, Y is NR₄.

In any one of the embodiments described herein, Y is O.

In any one of the embodiments described herein, Y is S, SO, SO₂, or SO(═NRₐ).

In any one of the embodiments described herein, Y is NR₄, CMeR₄, or CHR₄.

In any one of the embodiments described herein, the structural moiety

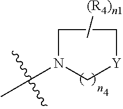

has the structure of

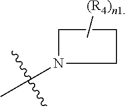

In any one of the embodiments described herein, the structural moiety

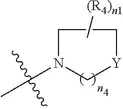

has the structure of

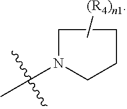

In any one of the embodiments described herein, the structural moiety

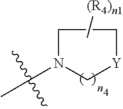

has the structure of

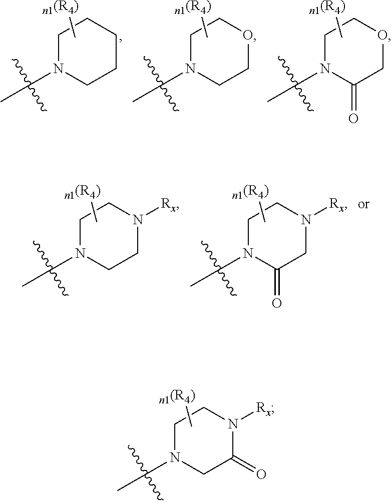

where Rₓ is R₄.

In any one of the embodiments described herein, the structural moiety

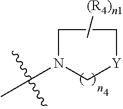

has the structure of

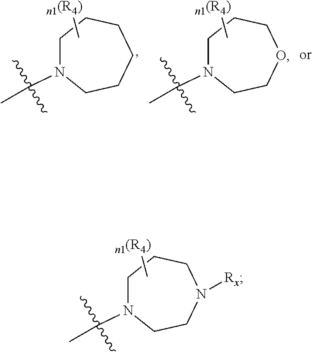

where Rₓ is R₄.

In any one of the embodiments described herein, R₁ and R₂ are each independently H or alkyl.

In any one of the embodiments described herein, R₁ and R₂ are each independently H or Me.

In any one of the embodiments described herein, R₁ and R₂ are each independently H, (CR₆R₇)ₙ₃ORₐ, (CR₆R₇)ₙ₃NRₐR_{b}, (CR₆R₇)ₙ₃(C═O)NR_{b}Rₐ, or (CR₆R₇)ₙ₃NR_{b}(C═O)Rₐ.

In any one of the embodiments described herein, R₁ and R₂ are each independently H, CH₂OH, CH₂NH₂, or CONH₂.

In any one of the embodiments described herein, at least one occurrence of R₄ is independently (CR₆R₇)ₙ₃ORₐ, (CR₆R₇)ₙ₃NRₐR_{b}, (CR₆R₇)ₙ₃SO₂Rₐ, (CR₆R₇)ₙ₃NRₐ(C═O)R_{b}, or (CR₆R₇)ₙ₃(C═O)NRₐR_{b}.

In any one of the embodiments described herein, at least one occurrence of R₄ is independently (CR₆R₇)ₙ₃NRₐ(C═O)R_{b} or (CR₆R₇)ₙ₃(C═O)NRₐR_{b}.

In any one of the embodiments described herein, one or more occurrences of R₄ are (CR₆R₇)ₙ₃ORₐ or (CR₆R₇)ₙ₃NRₐR_{b}.

In any one of the embodiments described herein, one or more occurrences of R₄ are ORₐ, NRₐR_{b}, —CH₂ORₐ, —CH₂NRₐR_{b}, —CH₂CH₂ORₐ, or —CH₂CH₂NRₐR_{b}.

In any one of the embodiments described herein, at least one occurrence of R₄ is an optionally substituted 5- or 6-membered heterocycle containing 1-3 heteroatoms each selected from the group consisting of N, O, and S.

In any one of the embodiments described herein, two R₄ taken together forming an optionally substituted carbocycle, saturated heterocycle, or heteroaryl containing 0-3 heteroatoms each selected from the group consisting of N, O, and S.

In any one of the embodiments described herein, at least one occurrence of R₄ is CH₂OH, CH₂NH₂,

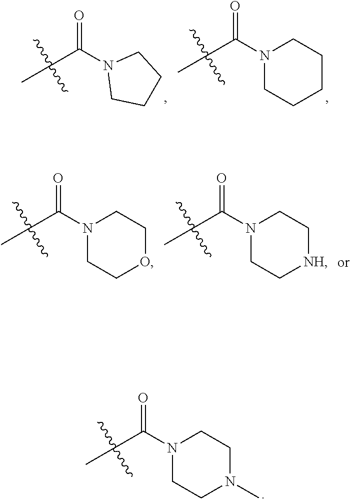

In any one of the embodiments described herein, at least one occurrence of R₄ is a heterocycle selected from the group consisting of

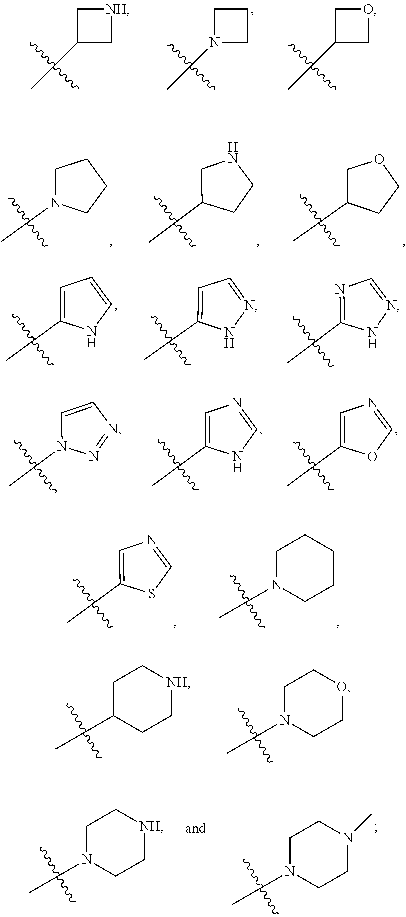

where the heterocycle is optionally substituted by alkyl, OH, oxo, or (C═O)C₁₋₄alkyl where valence permits.

In any one of the embodiments described herein, at least one occurrence of R₄ is H, alkyl, cycloalkyl, optionally substituted saturated heterocycle, optionally substituted aryl, optionally substituted heteroaryl, CN, CF₃, OCF₃, ORₐ, (CR₆R₇)ₙ₃ORₐ, or oxo.

In any one of the embodiments described herein, at least one occurrence of R₄ is (C═O)R_{b}, (C═O)OR_{b}, SO₂Rₐ, (C═O)(CR₆R₇)ₙ₃OR_{b}, (C═O)(CR₆R₇)ₙ₃NRₐR_{b}, (CR₆R₇)ₙ₃NRₐR_{b}, (CR₆R₇)ₙ₃NRₐSO₂R_{b}, (CR₆R₇)ₙ₃NRₐ(C═O)R_{b}, (CR₆R₇)ₙ₃NRₐ(C═O)NRₐR_{b}, or (CR₆R₇)ₙ₃(C═O)NRₐR_{b}.

In any one of the embodiments described herein, at least one occurrence of R₄ is independently H or alkyl.

In any one of the embodiments described herein, two R₄ groups taken together with the carbon atom that they are connected to form a 3-7 membered optionally substituted carbocycle or heterocycle.

In any one of the embodiments described herein, two R₄ groups taken together with the two carbon atoms that they are connected to form a fused bicyclic system having the structure of

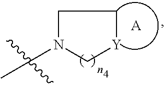

where A is a 3-7 membered optionally substituted carbocycle, saturated heterocycle, or heteroaryl.

In any one of the embodiments described herein, the structural motif

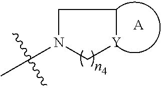

has the structure of

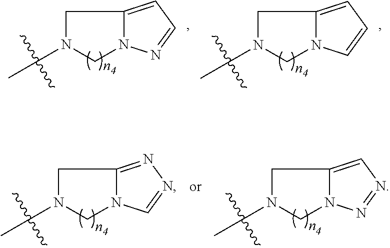

In any one of the embodiments described herein, each occurrence of R₆ and R₇ are independently H or alkyl.

In any one of the embodiments described herein, Z is OH or OMe.

In any one of the embodiments described herein, Z is OH.

In any one of the embodiments described herein, X₁ is H, CN, halogen, fluorinated alkyl, or alkyl.

In any one of the embodiments described herein, X₁ is H, CN, Cl, Br, Me, or CF₃.

In any one of the embodiments described herein, X₁ is H or Cl.

In any one of the embodiments described herein, X₂ is H, CN, halogen, fluorinated alkyl, or alkyl.

In any one of the embodiments described herein, X₂ is H, CN, Cl, Br, Me, or CF₃.

In any one of the embodiments described herein, X₂ is H or Cl.

In any one of the embodiments described herein, X₃ is H, halogen, CN, alkyl, or halogenated alkyl.

In any one of the embodiments described herein, X₃ is H, Cl, Br, Me, or CF₃.

In any one of the embodiments described herein, X₃ is H or Cl.

In any one of the embodiments described herein, the structural moiety

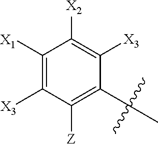

has the structure of

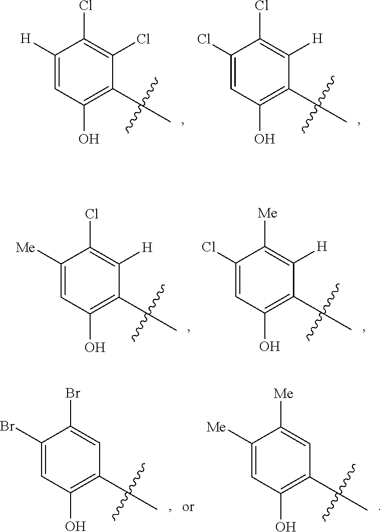 [SMILES: *c1c(O)ccc(Cl)c1Cl ; *c1cc(Cl)c(Cl)cc1O ; *c1cc(Cl)c(C)cc1O ; *c1cc(C)c(Cl)cc1O ; *c1cc(Br)c(Br)cc1O ; *c1cc(C)c(C)cc1O]

In any one of the embodiments described herein, n₁ is 0, 1, 2, or 3.

In any one of the embodiments described herein, each occurrence of n₃ is independently 0, 1, or 2.

In any one of the embodiments described herein, n₄ is 1 or 2.

In any one of the embodiments described herein, at least one occurrence of Rₐ or R_{b} is independently H, alkyl, cycloalkyl, saturated heterocycle, aryl, or heteroaryl.

In any one of the embodiments described herein, at least one occurrence of Rₐ or R_{b} is independently H, Me, Et, Pr, or a heterocycle selected from the group consisting of

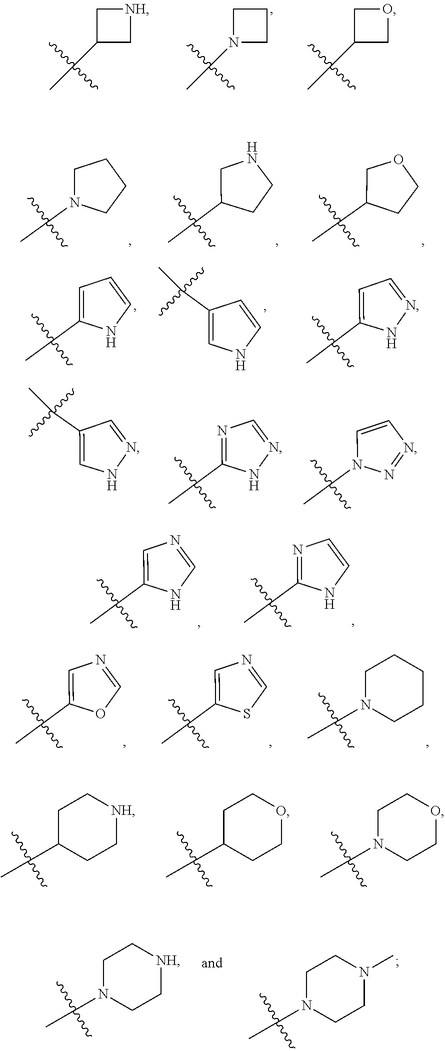

where the heterocycle is optionally substituted by alkyl, OH, oxo, or (C═O)C₁₋₄alkyl where valence permits.

In any one of the embodiments described herein, Rₐ and R_{b} together with the nitrogen atom that they are connected to form an optionally substituted heterocycle including the nitrogen atom and 0-3 additional heteroatoms each selected from the group consisting of N, O, and S.

In any one of the embodiments described herein, the compound is selected from the group consisting of compounds 1-66 as shown in Table 1.

In another aspect, a pharmaceutical composition is described, including at least one compound according to any one of the embodiments described herein or a pharmaceutically acceptable salt thereof and a pharmaceutically acceptable carrier or diluent.

In yet another aspect, a method of treating a condition in a mammalian species in need thereof is described, including administering to the mammalian species a therapeutically effective amount of at least one compound according to any one of the embodiments described herein or a pharmaceutically acceptable salt thereof, where the condition is selected from the group consisting of cancer, an immunological disorder, a Central Nerve System (CNS) disorder, an inflammatory disorder, a gastroenterological disorder, a metabolic disorder, a cardiovascular disorder, and a kidney disease.

In any one of the embodiments described herein, the immunological disorder is transplant rejection or an autoimmune disease.

In any one of the embodiments described herein, the autoimmune disease is rheumatoid arthritis, multiple sclerosis, systemic lupus erythematosus, or Type I diabetes mellitus.

In any one of the embodiments described herein, the Central Nerve System (CNS) disorder is Alzheimer's disease.

In any one of the embodiments described herein, the inflammatory disorder is an inflammatory skin condition, arthritis, psoriasis, spondylitis, parodontitis, or an inflammatory neuropathy.

In any one of the embodiments described herein, the gastroenterological disorder is an inflammatory bowel disease.

In any one of the embodiments described herein, the metabolic disorder is obesity or Type II diabetes mellitus.

In any one of the embodiments described herein, the cardiovascular disorder is an ischemic stroke.

In any one of the embodiments described herein, the kidney disease is chronic kidney disease, nephritis, or chronic renal failure.

In any one of the embodiments described herein, the condition is selected from the group consisting of cancer, transplant rejection, rheumatoid arthritis, multiple sclerosis, systemic lupus erythematosus, Type I diabetes mellitus, Alzheimer's disease, inflammatory skin condition, inflammatory neuropathy, psoriasis, spondylitis, parodontitis, Crohn's disease, ulcerative colitis, obesity, Type II diabetes mellitus, ischemic stroke, chronic kidney disease, nephritis, chronic renal failure, and a combination thereof.

In any one of the embodiments described herein, the mammalian species is human.

In yet another aspect, a method of blocking Kv1.3 potassium channel in a mammalian species in need thereof is described, including administering to the mammalian species a therapeutically effective amount of at least one compound according to any one of the embodiments described herein or a pharmaceutically acceptable salt thereof.

In any one of the embodiments described herein, the mammalian species is human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited, to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited, to one or more of the following groups: hydrogen, halogen, alkyl, halogenated alkyl (i.e., an alkyl group bearing a single halogen substituent or multiple halogen substituents such as CF₃ or CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary such groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NR_{d}S(═O)₂NR_{b}R_{c}, NRₐP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings wherein two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NR_{d}S(═O)₂NR_{b}R_{c}, NRₐP(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl or naphthyl. The term “carbocycle” encompasses cycloalkyl, cycloalkenyl, cycloalkynyl and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms and sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, indolinyl, isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “oxo” refers to

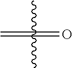 [SMILES: *=O]

substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be re-arranged to satisfy the valence requirement. For instance, a pyridine with a 2-oxo substituent group may have the structure of

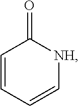

which also includes its tautomeric form of

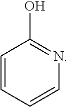

The term “alkylamino” refers to a group having the structure —NHR′, wherein R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,2,4-triazolyl, and tetrazolyl.

The terms “halogen” or “halo” refer to chlorine, bromine, fluorine or iodine.

The term “substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such but not limited to a phenol or carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention. As used herein, any depicted structure of the compound includes the tautomeric forms thereof.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999), the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements and, hence, may be preferred in some circumstances. Isotopically labeled compounds can generally be prepared by carrying out the procedures disclosed in the Schemes and/or in the Examples below, by substituting a readily available isotopically labeled reagent for a non-isotopically labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; Cardio-facio-cutaneous syndrome; Hereditary gingival fibromatosis type 1; Autoimmune lymphoproliferative syndrome; and capillary malformation-arterovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

### Compounds

Novel compounds as Kv1.3 potassium channel blockers are described. Applicants have surprisingly discovered that the compounds disclosed herein exhibit potent Kv1.3 potassium channel-inhibiting properties. Additionally, Applicants have surprisingly discovered that the compounds disclosed herein selectively block the Kv1.3 potassium channel and do not block the hERG channel and thus have desirable cardiovascular safety profiles.

In one aspect, a compound of Formula I or a pharmaceutically acceptable salt thereof is described,

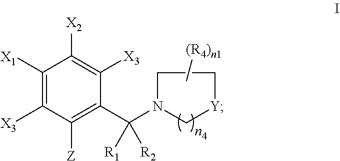

wherein

each occurrence of Y is independently C(R₄)₂, NR₄, O, S, SO, SO₂, or SO(═NRₐ);

Z is ORₐ;

X₁ is H, halogen, CN, alkyl, halogenated alkyl, cycloalkyl, or halogenated cycloalkyl;

X₂ is H, halogen, CN, alkyl, halogenated alkyl, cycloalkyl, or halogenated cycloalkyl;

each occurrence of X₃ is independently H, halogen, CN, alkyl, halogenated alkyl, cycloalkyl, or halogenated cycloalkyl;

R₁ and R₂ are each independently H, alkyl, (CR₆R₇)ₙ₃ORₐ, (CR₆R₇)ₙ₃NRₐR_{b}, (CR₆R₇)ₙ₃(C═O)NR_{b}Rₐ, or (CR₆R₇)ₙ₃NR_{b}(C═O)Rₐ;

each occurrence of R₄ is independently H, halogen, alkyl, cycloalkyl, halogenated alkyl, halogenated cycloalkyl, optionally substituted saturated heterocycle, optionally substituted aryl, optionally substituted heteroaryl, CN, oxo, (C═O)R_{b}, (C═O)OR_{b}, (CR₆R₇)ₙ₃ORₐ, (CR₆R₇)ₙ₃NRₐR_{b}, (CR₆R₇)ₙ₃SO₂Rₐ, (CR₆R₇)ₙ₃SO₂NRₐR_{b}, (CR₆R₇)ₙ₃NRₐSO₂R_{b}, (CR₆R₇)ₙ₃NRₐ(C═O)R_{b}, (CR₆R₇)ₙ₃(C═O)NRₐR_{b}, (CR₆R₇)ₙ₃NRₐ(C═O)NRₐR_{b}, (C═O)(CR₆R₇)ₙ₃OR_{b}, (C═O)(CR₆R₇)ₙ₃NRₐR_{b}, or an optionally substituted 5- or 6-membered heterocycle containing 1-3 heteroatoms each selected from the group consisting of N, O, and S;

or two R₄ taken together forming an optionally substituted carbocycle, saturated heterocycle, or heteroaryl containing 0-3 heteroatoms each selected from the group consisting of N, O, and S;

each occurrence of R₆ and R₇ are independently H, alkyl, cycloalkyl, optionally substituted aryl, or optionally substituted heteroaryl;

each occurrence of Rₐ and R_{b} are independently H, alkyl, alkenyl, cycloalkyl, halogenated alkyl, halogenated cycloalkyl, optionally substituted saturated heterocycle, optionally substituted aryl, or optionally substituted heteroaryl; or alternatively Rₐ and R_{b} together with the nitrogen atom that they are connected to form an optionally substituted heterocycle comprising the nitrogen atom and 0-3 additional heteroatoms each selected from the group consisting of N, O, and S;

the alkyl, cycloalkyl, carbocycle, heterocycle, aryl, and heteroaryl are optionally substituted by 1-4 substituents each independently selected from the group consisting of alkyl, cycloalkyl, halogenated cycloalkyl, halogenated alkyl, halogen, (CR₆R₇)ₙ₃ORₐ, (CR₆R₇)ₙ₃NRₐR_{b}, (CR₆R₇)ₙ₃NRₐ(C═O)R_{b}, (CR₆R₇)ₙ₃(C═O)NRₐR_{b}, and oxo where valence permits;

each occurrence of n₁ is independently an integer from 0-4 where valence permits;

each occurrence of n₃ is independently an integer from 0-4; and

each occurrence of n₄ is independently 0, 1 or 2.

In some embodiments, n₁ is an integer from 1-4. In some embodiments, n₁ is an integer from 1-3. In some embodiments, n₁ is 1 or 2. In some embodiments, n₁ is 1. In some embodiments, n₁ is 0.

In some embodiments, n₃ is an integer from 0-4. In some embodiments, n₃ is an integer from 1-3. In some embodiments, n₃ is 0. In some embodiments, n₃ is 1 or 2. In some embodiments, n₃ is 1.

In some embodiments, n₄ is an integer from 0-2. In some embodiments, n₄ is 0. In some embodiments, n₄ is 2. In some embodiments, n₄ is 1.

In some embodiments, the structural moiety

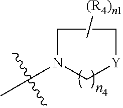

has the structure of

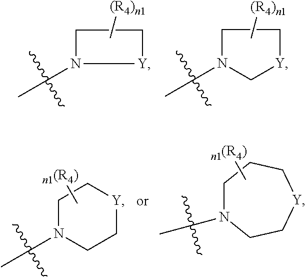

wherein the various substituents are defined herein. In some embodiments, the structural moiety

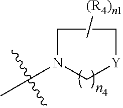

has the structure of

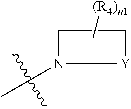

In some embodiments, the structural moiety

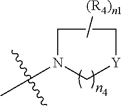

has the structure of

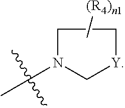

In some embodiments, the structural moiety

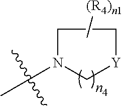

has the structure of

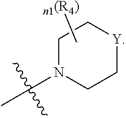

In some embodiments, the structural moiety

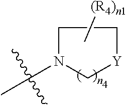

has the structure of

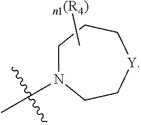

In some embodiments, Y is C(R₄)₂. In other embodiments, Y is NR₄. In still other embodiments, Y is O. In still other embodiments, Y is S, SO, SO₂, or SO(═NRₐ). In some specific embodiments, Y is NR₄, CMeR₄, or CHR₄. In some specific embodiments, Y is NH. In some specific embodiments, Y is CH₂.

In some embodiments, the structural moiety

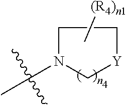

has the structure of

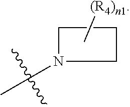

In other embodiments, the structural moiety

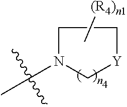

has the structure of

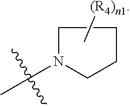

In still other embodiments, the structural moiety

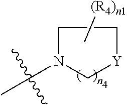

has the structure of

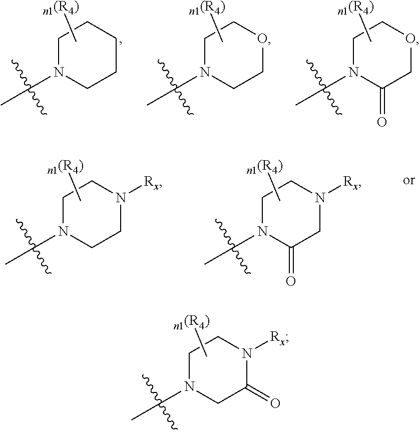

wherein Rₓ is R₄.
In some specific embodiments, the structural moiety

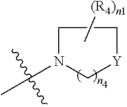

has the structure of

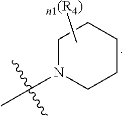

In some specific embodiments, the structural moiety

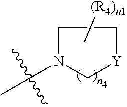

has the structure of

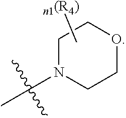

In some embodiments, the structural moiety

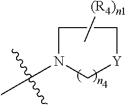

has the structure of

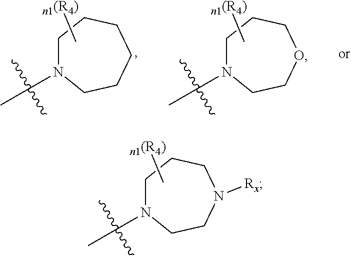

wherein Rₓ is R₄. In some specific embodiments, the structural moiety

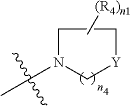

has the structure of

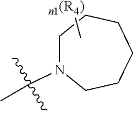

In some specific embodiments, the structural moiety

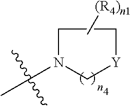

has the structure of

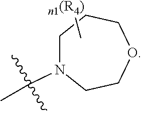

In some specific embodiments, the structural moiety

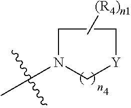

has the structure of

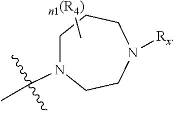

In some embodiments, R₁ and R₂ are each H or alkyl. In some embodiments, R₁ and R₂ are both H. In some embodiments, R₁ and R₂ are alkyl, such as Me, Et, propyl, isopropyl, n-butyl, iso-butyl, or sec-butyl. In some embodiments, R₁ and R₂ are H and alkyl, respectively.

In some embodiments, at least one occurrence of R₁ and R₂ is (CR₆R₇)ₙ₃ORₐ or (CR₆R₇)ₙ₃NRₐR_{b}. In some embodiments, at least one occurrence of R₁ and R₂ is H.

In some embodiments, R₁ and R₂ are each independently H, (CR₆R₇)ₙ₃ORₐ, (CR₆R₇)ₙ₃NRₐR_{b}, (CR₆R₇)ₙ₃(C═O)NR_{b}Rₐ, or (CR₆R₇)ₙ₃NR_{b}(C═O)Rₐ. In some specific embodiments, R₁ and R₂ are each independently H, Me, CH₂OH, CH₂NH₂, CONH₂, CONHMe₂, CONMe₂, NH(CO)Me, or NMe(CO)Me. In some embodiments, R₁ and R₂ are each independently H, CH₂OH, CH₂NH₂, or CONH₂. In other embodiments, R₁ and R₂ are each independently selected from the group consisting of H and Me.

In some embodiments, at least one occurrence of R₄ is independently (CR₆R₇)ₙ₃ORₐ, (CR₆R₇)ₙ₃NRₐR_{b}, (CR₆R₇)ₙ₃SO₂Rₐ, (CR₆R₇)ₙ₃NRₐ(C═O)R_{b}, or (CR₆R₇)ₙ₃(C═O)NRₐR_{b}. In some embodiments, at least one occurrence of R₄ is independently (CR₆R₇)ₙ₃NRₐ(C═O)R_{b} or (CR₆R₇)ₙ₃(C═O)NRₐR_{b}. In some embodiments, at least one occurrence of R₄ is independently (CR₆R₇)ₙ₃ORₐ or (CR₆R₇)ₙ₃NRₐR_{b}. In some embodiments, at least one occurrence of R₄ is independently ORₐ, NRₐR_{b}, —CH₂ORₐ, —CH₂NRₐR_{b}, —CH₂CH₂ORₐ, or —CH₂CH₂NRₐR_{b}. In some specific embodiments, R₄ is NH₂, CH₂NH₂, CH₂CH₂NH₂, CONH₂, CONHMe₂, CONMe₂, NH(CO)Me, NMe(CO)Me, CH₂CONH₂, CH₂CONHMe₂, CH₂CONMe₂, CH₂NH(CO)Me, or CH₂NMe(CO)Me. In other specific embodiments, at least one occurrence of R₄ is CH₂NH₂,

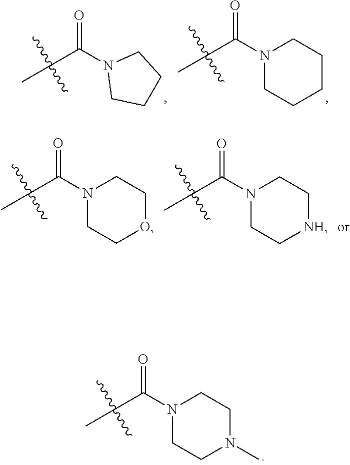

In other specific embodiments, at least one occurrence of R₄ is CH₂OH, CH₂NH₂,

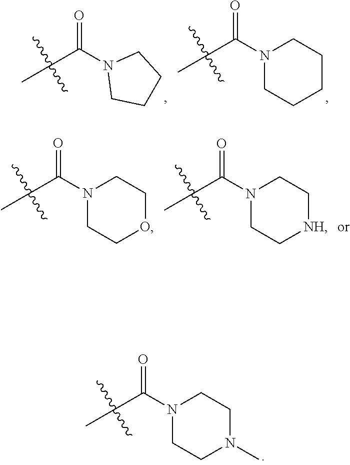

In still other embodiments, at least one occurrence of R₄ is an optionally substituted 4-, 5- or 6-membered heterocycle containing 1-3 heteroatoms each selected from the group consisting of N, O, and S. In further embodiments, at least one occurrence of R₄ is a heterocycle selected from the group consisting of

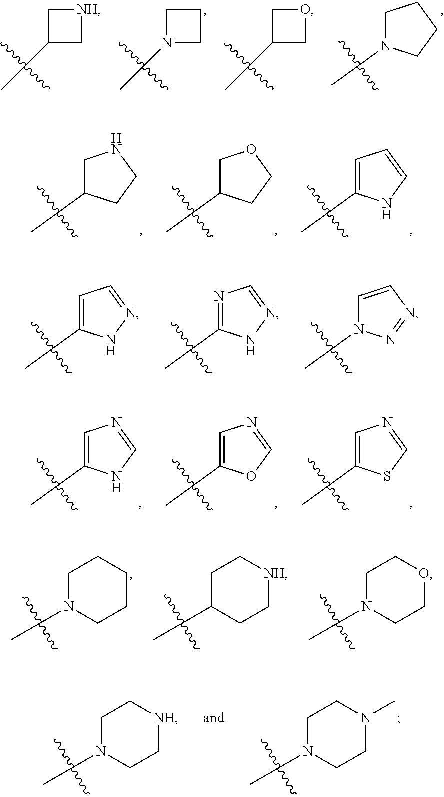

wherein the heterocycle is optionally substituted by alkyl, OH, oxo, or (C═O)C₁₋₄alkyl where valence permits. In further embodiments, two R₄ taken together forming an optionally substituted carbocycle, saturated heterocycle, or heteroaryl containing 0-3 heteroatoms each selected from the group consisting of N, O, and S.

In some embodiments, at least one occurrence of R₄ is H, alkyl, cycloalkyl, optionally substituted saturated heterocycle, optionally substituted aryl, optionally substituted heteroaryl, CN, CF₃, OCF₃, ORₐ, (CR₆R₇)ₙ₃ORₐ, or oxo. In some embodiments, at least one occurrence of R₄ is (C═O)R_{b}, (C═O)OR_{b}, SO₂Rₐ, (C═O)(CR₆R₇)ₙ₃OR_{b}, (C═O)(CR₆R₇)ₙ₃NRₐR_{b}, (CR₆R₇)ₙ₃NRₐR_{b}, (CR₆R₇)ₙ₃NRₐSO₂R_{b}, (CR₆R₇)ₙ₃NRₐ(C═O)R_{b}, (CR₆R₇)ₙ₃NRₐ(C═O)NRₐR_{b}, or (CR₆R₇)ₙ₃(C═O)NRₐR_{b}.

In some specific embodiments, at least one occurrence of R₄ is H, halogen, alkyl, OH, NH₂, CN, CF₃, OCF₃, CONH₂, CONHMe₂, or CONMe₂. In some specific embodiments, R₄ is H, halogen, alkyl, cycloalkyl, CN, CF₃, ORₐ, (CR₆R₇)ₙ₃ORₐ, (C═O)OR_{b}, (C═O)(CR₆R₇)ₙ₃OR_{b}, (C═O)(CR₆R₇)ₙ₃NRₐR_{b}, (CR₆R₇)ₙ₃NRₐR_{b}, (CR₆R₇)ₙ₃NRₐ(C═O)R_{b}, (CR₆R₇)ₙ₃SO₂NRₐR_{b}, (CR₆R₇)ₙ₃SO₂Rₐ, oxo, or (CR₆R₇)ₙ₃(C═O)NRₐR_{b}. In some embodiments, at least one occurrence of R₄ is independently H or alkyl.

In some specific embodiments, R₄ is H, halogen, alkyl, ORₐ, NRₐR_{b}, or oxo. In other specific embodiments, R₄ is H, F, Cl, Br, Me, Et, Pr, iso-Pr, Bu, iso-Bu, sec-Bu, or tert-Bu. In other specific embodiments, R₄ is OH, NH₂, NHMe, NMe₂, NHEt, NMeEt, NEt₂, or oxo. In still other specific embodiments, at least one occurrence of R₄ is H, halogen, alkyl, OH, NH₂, CN, CF₃, OCF₃, CONH₂, CONHMe₂, or CONMe₂.

In other embodiments, two R₄ groups taken together with the two carbon atoms that they are connected to form a fused bicyclic system having the structure of

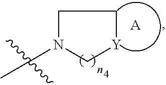

wherein A is a 3-7 membered optionally substituted carbocycle, saturated heterocycle, or heteroaryl. In some embodiments, the structural motif

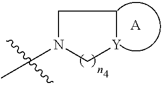

has the structure of

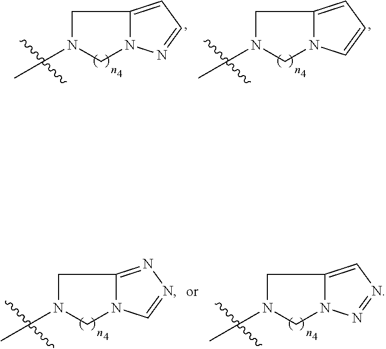 [SMILES: *N1Cc2ccnn2C1 ; *N1Cc2cccn2C1 ; *N1Cc2nncn2C1 ; *N1Cc2cnnn2C1]

In some embodiments, each occurrence of R₆ and R₇ are independently H or alkyl. In some specific embodiments, CR₆R₇ is CH₂, CHMe, CMe₂, CHEt, or CEt₂. In some specific embodiments, CR₆R₇ is CH₂.

In some embodiments, Z is ORₐ. In some embodiments, Z is OH, or OMe. In some embodiments, Z is OH.

In some embodiments, X₁ is H, halogen, CN, alkyl, halogenated alkyl, cycloalkyl, or halogenated cycloalkyl. In any one of the embodiments described herein, X₁ may be H, halogen, fluorinated alkyl, or alkyl. In some embodiments, X₁ is H or halogen. In other embodiments, X₁ is fluorinated alkyl or alkyl. In other embodiments, X₁ is cycloalkyl. In some embodiments, X₁ is H, F, Cl, Br, Me, or CF₃. In some embodiments, X₁ is H, F, or Cl. In some embodiments, X₁ is F or Cl. In some embodiments, X₁ is H or Cl. In some embodiments, X₁ is F. In some embodiments, X₁ is CF₃.

In some embodiments, X₂ is H, halogen, CN, alkyl, halogenated alkyl, cycloalkyl, or halogenated cycloalkyl. In any one of the embodiments described herein, X₂ may be H, halogen, fluorinated alkyl, or alkyl. In some embodiments, X₂ is H or halogen. In other embodiments, X₂ is fluorinated alkyl or alkyl. In other embodiments, X₂ is cycloalkyl. In some embodiments, X₂ is H, F, Cl, Br, Me, or CF₃. In some embodiments, X₂ is H, F, or Cl. In some embodiments, X₂ is F or Cl. In some embodiments, X₂ is H or Cl. In some embodiments, X₂ is F. In some embodiments, X₂ is CF₃.

In some embodiments, each occurrence of X₃ is independently H, halogen, CN, alkyl, halogenated alkyl, cycloalkyl, or halogenated cycloalkyl. In any one of the embodiments described herein, X₃ may be H, halogen, fluorinated alkyl, or alkyl. In some embodiments, X₃ is H or halogen. In other embodiments, X₃ is fluorinated alkyl or alkyl. In other embodiments, X₃ is cycloalkyl. In some embodiments, X₃ is H, F, Cl, Br, Me, or CF₃. In some embodiments, X₃ is H, F, or Cl. In some embodiments, X₃ is F or Cl. In some embodiments, X₃ is H or Cl. In some embodiments, X₃ is F. In some embodiments, X₃ is CF₃.

In some embodiments, the structural moiety

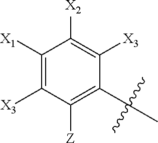

has the structure of

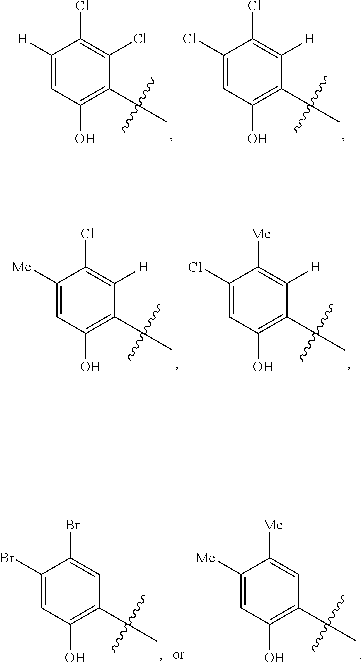 [SMILES: *c1c(O)ccc(Cl)c1Cl ; *c1cc(Cl)c(Cl)cc1O ; *c1cc(Cl)c(C)cc1O ; *c1cc(C)c(Cl)cc1O ; *c1cc(Br)c(Br)cc1O ; *c1cc(C)c(C)cc1O]

In some embodiments, Z is OH or OMe. In some embodiments, Z is OH.

In any one of the embodiments described herein, at least one occurrence of Rₐ or R_{b} is independently H or optionally substituted alkyl, cycloalkyl, saturated heterocycle, aryl, or heteroaryl. In some embodiments, at least one occurrence of Rₐ or R_{b} is independently H, Me, Et, Pr, or Bu. In some embodiments, at least one occurrence of Rₐ or R_{b} is independently a heterocycle selected from the group consisting of

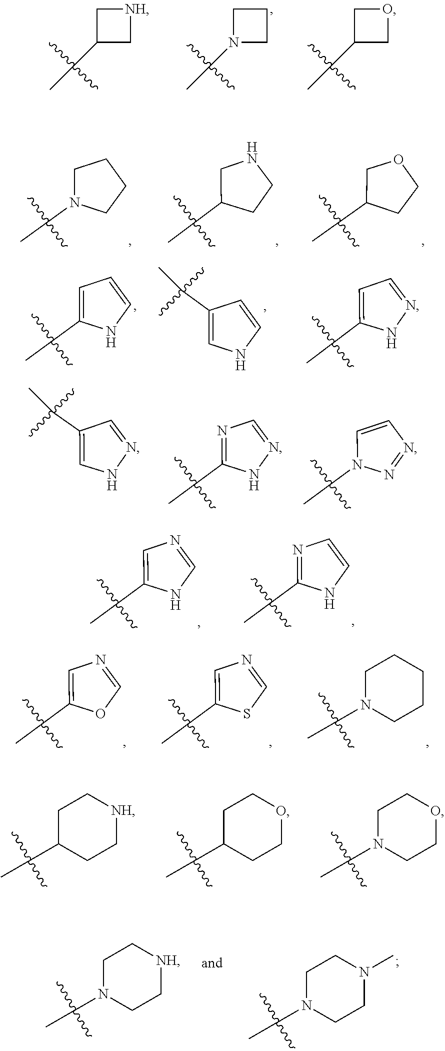

wherein the heterocycle is optionally substituted by alkyl, OH, oxo, or (C═O)C₁₋₄alkyl where valence permits.

In some embodiments, Rₐ and R_{b} together with the nitrogen atom that they are connected to form an optionally substituted heterocycle comprising the nitrogen atom and 0-3 additional heteroatoms each selected from the group consisting of N, O, and S.

In some embodiments, the compound of Formula I is selected from the group consisting of compounds 1-66 as shown in Table 1 below.

### Abbreviations

ACN Acetonitrile
Boc tert-Butyloxycarbonyl
DCE Dichloroethane
DCM Dichloromethane
DIBAL-H Diisobutylaluminium hydride
DMF Dimethyl formamide
EA Ethyl acetate
HATU N-[(dimethylamino)(3H-1,2,3-triazolo(4,4-b)pyridin-3-yloxy)methylene]-N-methylmethanaminium hexafluorophosphate
LDA Lithium diisopropylamide
PE Petroleum ether
PMHS Polymethylhydrosiloxane
TEA Triethylamine
TFA Trifluoroacetic acid
THF Tetrahydrofuran

### Methods of Preparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations, but not limitations of the preparation of some of the starting materials and compounds disclosed herein.

Schemes 1-3 below describe synthetic routes which may be used for the synthesis of compounds of the present invention, e.g., compounds having a structure of Formula I or a precursor thereof. Various modifications to these methods may be envisioned by those skilled in the art to achieve similar results to that of the inventions given below. In the embodiments below, the synthetic route is described using compounds having the structure of Formula I or a precursor thereof as examples. The general synthetic routes described in Schemes 1-3, and examples described in the Example section below, illustrate methods used for the preparation of the compounds described herein.

Compounds I-1 and I-3, as shown immediately below in Scheme 1, can be prepared by any method known in the art and/or are commercially available. The substituents shown in Scheme 1 are defined herein. Compounds as disclosed herein where R₁ and R₂ are H can be made by reductive amination of an aryl aldehyde I-2a with a cyclic amine I-3 to give compound I-4a (Scheme 1). If not commercially available, the aldehyde I-2a can be obtained by formylation of a substituted benzene I-1 with paraformaldehyde, magnesium chloride, and a base such as TEA in a solvent such as ACN. The reductive amination of aryl aldehyde I-2a with a cyclic amine I-3 may be carried out with a reducing agent such as sodium triacetoxy borohydride in a solvent such as DCE, or with PMHS and tin chloride in a solvent such as methanol. For compounds disclosed herein where Z is OH, no protecting group is necessary for the reductive amination step. For compounds disclosed herein where R₄ contains an amino group, the amine may be protected with a protecting group, e.g., Boc or trifluoroacetamide. Any other protecting groups for amine known in the art can be used. The protecting group is then removed after the reductive amination step.

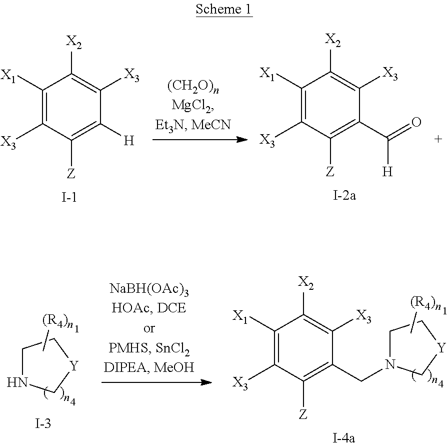

Compounds I-2c and I-3, as shown immediately below in Scheme 2, can be prepared by any method known in the art and/or are commercially available. As shown in Scheme 2, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. The substituents shown in Scheme 2 are defined herein. Compounds disclosed herein where R₁ is an alkyl group can be prepared from benzaldehyde I-2c by reaction with a Grignard reagent R₁MgBr. The resulting alcohol I-5 is then converted to bromide I-6 with a bromination agent, e.g., phosphorus tribromide. Reaction of I-6 with cyclic amine I-3 in the presence of a base such as potassium carbonate in a solvent such as DMF gives I-4c (Scheme 2). In some embodiments, this method can also be used for compounds where R₁ and R₂ are both H.

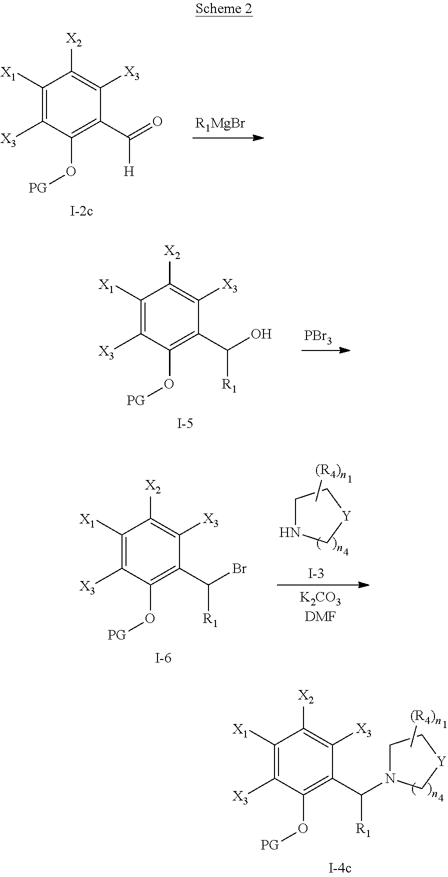

Compounds I-1b and I-3, as shown immediately below in Scheme 3, can be prepared by any method known in the art and/or are commercially available. As shown in Scheme 3, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. The substituents shown in Scheme 3 are defined herein. Compounds disclosed herein where R₁ is a functional group can be synthesized from phenol I-1b as shown in Scheme 3. Reaction of phenol I-1b with ethyl glyoxalate in the presence of a Lewis acid such as titanium tetrachloride in a solvent such as DCM gives alcohol I-5b. The phenol group in I-5b is then selectively protected, e.g., as an ether such as a methyl or allyl ether I-5c. I-5c is converted to bromide I-6b using a bromination agent such as phosphorus tribromide in a solvent such as DCM. Reaction of I-6b with amine I-3 provides I-4d. The ester group in I-4d can be converted to a variety of R₁ groups such as amide, hydroxymethyl or aminomethyl using methods known in the art. The protecting group PG can be optionally removed to afford a compound of Formula I.

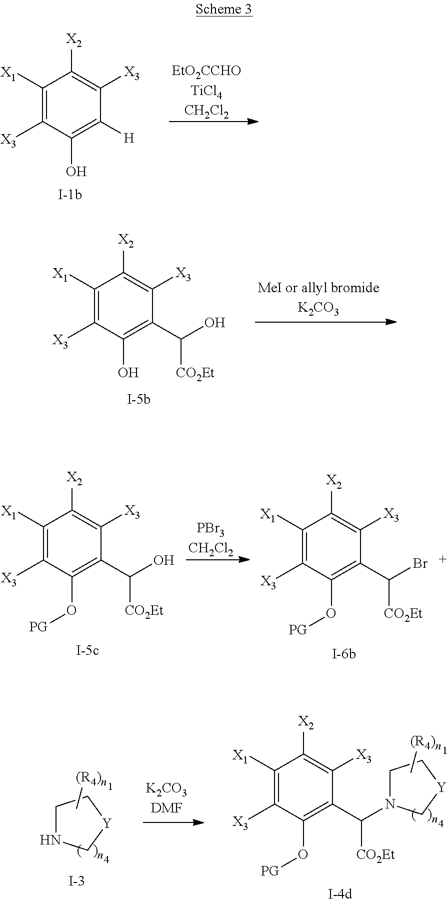

The reactions described above in Schemes 1-3 can be carried out in a suitable solvent. Suitable solvents include, but are not limited to, ACN, methanol, ethanol, DCM, DMF, THF, MTBE, or toluene. The reactions described in Schemes 1-3 may be conducted under inert atmosphere, e.g., under nitrogen or argon, or the reaction may be carried out in a sealed tube. The reaction mixture may be heated in a microwave or heated to an elevated temperature. Suitable elevated temperatures include, but are not limited to, 40, 50, 60, 80, 90, 100, 110, 120° C. or higher or the refluxing/boiling temperature of the solvent used. The reaction mixture may alternatively be cooled in a cold bath at a temperature lower than room temperature, e.g., 0, −10, −20, −30, −40, −50, −78, or −90° C. The reaction may be worked up by removing the solvent or partitioning of the organic solvent phase with one or more aqueous phases each optionally containing NaCl, NaHCO₃, or NH₄Cl. The solvent in the organic phase can be removed by reduced vacuum evaporation and the resulting residue may be purified using a silica gel column or HPLC.

### Pharmaceutical Compositions

This invention also provides a pharmaceutical composition comprising at least one of the compounds as described herein or a pharmaceutically acceptable salt or solvate thereof, and a pharmaceutically acceptable carrier.

In yet another aspect, the present invention provides a pharmaceutical composition comprising at least one compound selected from the group consisting of compounds of Formula I as described herein and a pharmaceutically acceptable carrier or diluent.

In certain embodiments, the composition is in the form of a hydrate, solvate or pharmaceutically acceptable salt. The composition can be administered to the subject by any suitable route of administration, including, without limitation, oral and parenteral.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically acceptable salts. The term “pharmaceutically acceptable salt”, in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, for example, Berge et al., (1977) “Pharmaceutical Salts”, J. Pharm. Sci. 66:1-19.)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine, and the like. (See, for example, Berge et al., supra.)

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets, may be, made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving, or dispersing the pharmaceutical agents in the proper medium. Absorption enhancers can also be used to increase the flux of the pharmaceutical agents of the invention across the skin. The rate of such flux can be controlled, by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. One strategy for depot injections includes the use of polyethylene oxide-polypropylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), race horses, birds, lizards, and any other organism, which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Administration to a Subject

In yet another aspect, the present invention provides a method for treating a condition in a mammalian species in need thereof, the method comprising administering to the mammalian species a therapeutically effective amount of at least one compound selected from the group consisting of compounds of Formula I, or a pharmaceutically acceptable salt thereof, wherein the condition is selected from the group consisting of cancer, an immunological disorder, a central nerve system (CNS) disorder, an inflammatory disorder, a gastroenterological disorder, a metabolic disorder, a cardiovascular disorder, and a kidney disease.

In some embodiments, the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

In some embodiments, the inflammatory disorder is an inflammatory skin condition, arthritis, psoriasis, spondylitis, parodontitis, or an inflammatory neuropathy. In some embodiments, the gastroenterological disorder is an inflammatory bowel disease such as Crohn's disease or ulcerative colitis.

In some embodiments, the immunological disorder is transplant rejection or an autoimmune disease (e.g., rheumatoid arthritis, multiple sclerosis, systemic lupus erythematosus, or Type I diabetes mellitus). In some embodiments, the Central Nerve System (CNS) disorder is Alzheimer's disease.

In some embodiments, the metabolic disorder is obesity or Type II diabetes mellitus. In some embodiments, the cardiovascular disorder is an ischemic stroke. In some embodiments, the kidney disease is chronic kidney disease, nephritis, or chronic renal failure.

In some embodiments, the mammalian species is human.

In some embodiments, the condition is selected from the group consisting of cancer, transplant rejection, rheumatoid arthritis, multiple sclerosis, systemic lupus erythematosus, Type I diabetes mellitus, Alzheimer's disease, inflammatory skin condition, inflammatory neuropathy, psoriasis, spondylitis, parodontitis, inflammatory bowel disease, obesity, Type II diabetes mellitus, ischemic stroke, chronic kidney disease, nephritis, chronic renal failure, and a combination thereof.

In yet another aspect, a method of blocking Kv1.3 potassium channel in a mammalian species in need thereof is described, including administering to the mammalian species a therapeutically effective amount of at least one compound of Formula I, or a pharmaceutically acceptable salt thereof.

In some embodiments, the compounds described herein is selective in blocking the Kv 1.3 potassium channels with minimal or no off-target inhibition activities against other potassium channels, or against calcium or sodium channels. In some embodiments, the compounds described herein do not block the hERG channels and therefore have desirable cardiovascular safety profiles.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants, and even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33, which is incorporated herein by reference.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, depending on activity of the compound, manner of administration, purpose of the administration (i.e., prophylactic or therapeutic), nature and severity of the disorder, age and body weight of the patient, different doses may be necessary. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically acceptable salt. When used in medicine the salts should be pharmaceutically acceptable, but non-pharmaceutically acceptable salts can conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v); and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, Pa.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974, and 5,407,686. In addition, pump-based hardware delivery systems can be used, some of which are adapted for implantation.

### Assays for Effectiveness of Kv1.3 Potassium Channel Blockers

In some embodiments, the compounds as described herein are tested for their activities against Kv1.3 potassium channel. In some embodiments, the compounds as described herein are tested for their Kv1.3 potassium channel electrophysiology. In some embodiments, the compounds as described herein are tested for their hERG electrophysiology.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

Examples 1-2 describe various intermediates used in the syntheses of representative compounds of Formula I disclosed herein.

### Example 1. Intermediate 1 (4,5-dichloro-2-hydroxybenzaldehyde)

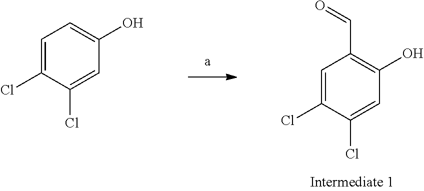 [SMILES: Oc1ccc(Cl)c(Cl)c1 ; O=Cc1cc(Cl)c(Cl)cc1O]

Step a:

To a stirred solution of 3,4-dichlorophenol (50.00 g, 306.75 mmol) in methanesulfonic acid (35 mL) was added hexamethylenetetramine (47.50 g, 337.40 mmol) at room temperature. The reaction solution was stirred at 110° C. for 30 min. The reaction solution was allowed to cool down to room temperature and quenched with water (500 mL). The resulting solution was extracted with DCM (3×500 mL) and dried over anhydrous Na₂SO₄. After the filtration, the filtrate was concentrated under reduced pressure. The residue was purified by silica gel column chromatography, eluted with PE/DCM (10/1) to afford Intermediate 1 (4,5-dichloro-2-hydroxybenzaldehyde) as a yellow solid (13.50 g, 23%): 1H NMR (400 MHz, CDCl3) δ 10.98 (s, 1H), 9.85 (s, 1H), 7.66 (s, 1H), 7.16 (s, 1H).

### Example 2. Intermediate 2 (1-(bromomethyl)-4,5-dichloro-2-methoxybenzene)

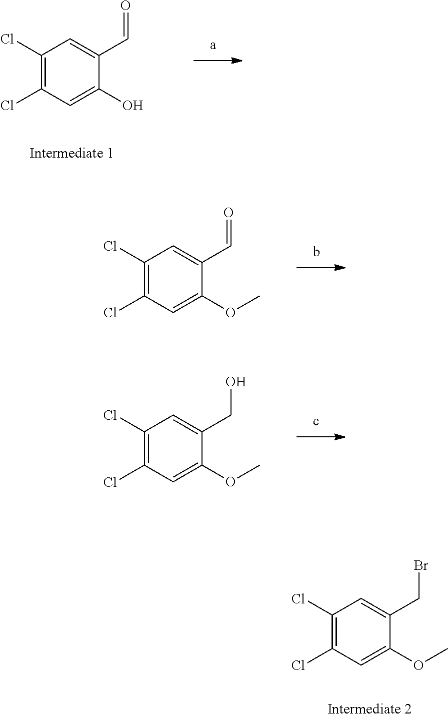

Step a:

To a stirred solution of Intermediate 1 (4,5-dichloro-2-hydroxybenzaldehyde) (10.00 g, 52.35 mmol) and K₂CO₃ (21.70 g, 157.06 mmol) in DMF (100 mL) was added CH₃I (11.10 g, 78.53 mmol) at room temperature. The resulting mixture was stirred at 30° C. for 2 h. The reaction was diluted with water (500 mL). The resulting mixture was extracted with EA (3×200 mL). The combined organic layers were washed with brine (3×200 mL) and dried over anhydrous Na₂SO₄. After filtration, the filtrate was concentrated under reduced pressure. The residue was purified by silica gel column chromatography, eluted with PE/EA (5/1) to afford 4,5-dichloro-2-methoxybenzaldehyde as an off-white solid (10.30 g, 96%): 1H NMR (300 MHz, CDCl₃) δ 10.32 (s, 1H), 7.85 (s, 1H), 7.08 (s, 1H), 3.91 (s, 3H).

Step b:

To a solution of 4,5-dichloro-2-methoxybenzaldehyde (5.00 g, 24.39 mmol) in EtOH (40 mL) and THE (5 mL) was added NaBH₄ (1.80 g, 48.88 mmol) at room temperature. After stirring for 1 h at room temperature, the resulting solution was quenched with water (1 mL) at room temperature and diluted with co-solvent of EA (80 mL) and water (100 mL). The isolated aqueous layer was extracted with EA (3×80 mL). The combined organic layer was washed with brine (3×80 mL) and dried over anhydrous Na₂SO₄. After filtration, the filtrate was concentrated under reduced pressure to afford (4,5-dichloro-2-methoxyphenyl)methanol as a light yellow solid (5.0. g, crude), which was used in next step without further purification.

Step c:

To a stirred solution of (4,5-dichloro-2-methoxyphenyl)methanol (5.00 g, 24.15 mmol) in CH₂Cl₂ (40 mL) was added PBr₃ (13.10 g, 48.30 mmol) at room temperature. After stirring for 1 h at room temperature, the resulting solution was quenched with water (80 mL). The aqueous layer was extracted with EA (3×80 mL). The combined organic layers were washed with brine (3×80 mL) and dried over anhydrous Na₂SO₄. After filtration, the filtrate was concentrated under reduced pressure. The residue was purified by silica gel column chromatography, eluted with PE/EA (4/1) to afford Intermediate 2 (1-(bromomethyl)-4,5-dichloro-2-methoxybenzene) as a light-yellow oil (5.00 g, 69%): 1H NMR (300 MHz, CDCl₃) δ 7.37 (s, 1H), 6.93 (s, 1H), 4.42 (s, 2H), 3.86 (s, 3H).

Examples 3-28 describe the syntheses of representative compounds of Formula I disclosed herein.

### Example 3. Compound 2 ((1-(4,5-dichloro-2-hydroxybenzyl)piperidine-2,4-diyl)dimethanol) Compound 1 (methyl 1-[(4,5-dichloro-2-hydroxyphenyl)methyl]-4-(hydroxymethyl)piperidine-2-carboxylate)

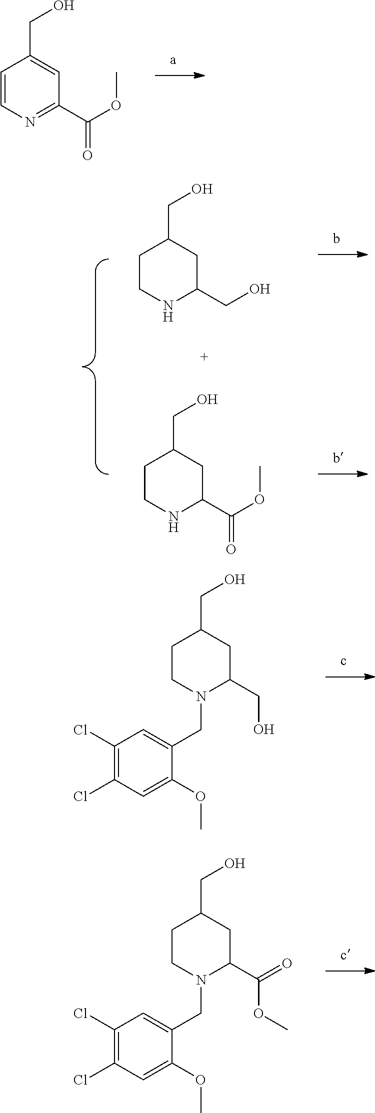

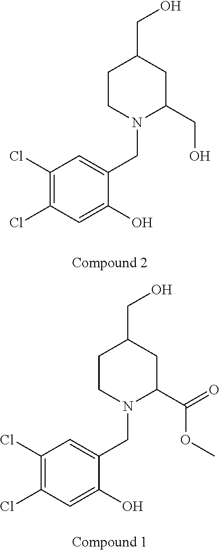 [SMILES: OCC1CCN(Cc2cc(Cl)c(Cl)cc2O)C(CO)C1 ; COC(=O)C1CC(CO)CCN1Cc1cc(Cl)c(Cl)cc1O]

Step a:

To a solution of methyl 4-(hydroxymethyl)pyridine-2-carboxylate (0.10 g, 0.60 mmol) in MeOH (5 mL) was added PtO₂ (10 mg, 10%) under nitrogen atmosphere at room temperature. The mixture was degassed with hydrogen three times. The mixture was stirred for 16 h at room temperature under hydrogen atmosphere (5 atm). The mixture was filtered. The filter cake was washed with MeOH (2×2 mL). The filtrate was concentrated under reduced pressure. The residue was purified by reverse phase chromatography, eluted with 40% ACN in water with 20 mM NH₄HCO₃. The faster-eluting was obtained as piperidine-2,4-diyldimethanol as a light yellow oil (0.2 g, 20%): LCMS (ESI) calculated for C₇H₁₅NO₂ [M+H]⁺: 146, found 146.

The slower-eluting was obtained as methyl 4-(hydroxymethyl)piperidine-2-carboxylate as a light yellow oil (0.30 g, 30%): LCMS (ESI) calculated for C₈H₁₅NO₃ [M+H]⁺: 174, found 174.

Step b:

To a mixture of piperidine-2,4-diyldimethanol (0.35 g, 2.04 mmol) and K₂CO₃ (0.51 g, 3.70 mmol) and in DMF (3 mL) was added Intermediate 2 (0.50 g, 1.85 mmol) at room temperature. The reaction mixture was allowed to warm to 45° C. and stirred for 2 h. After cooling to room temperature, the resulting mixture was diluted with water (20 mL) and extracted with EA (3×50 mL). The combined organic layers were washed with brine (3×20 mL) and dried over anhydrous Na₂SO₄. After filtration, the filtrate was concentrated under reduced pressure. The residue was purified by silica gel column chromatography, eluted with DCM/MeOH (10/1) to afford (1-(4,5-dichloro-2-methoxybenzyl)piperidine-2,4-diyl)dimethanol as a light-yellow oil (0.17 g, 28%): LCMS (ESI) calculated for C₁₅H₂₁C₁₂NO₃ [M+H]⁺: 334, 336 (3:2), found 334, 336 (3:2).

Step c:

To a solution of (1-(4,5-dichloro-2-methoxybenzyl)piperidine-2,4-diyl)dimethanol (0.15 g, 0.45 mmol) in DCM (1 mL) was added BBr₃ (0.56 g, 2.24 mmol) at room temperature. After stirring for 1 h at room temperature, the resulting mixture was quenched with saturated aq. NaHCO₃ (10 mL) at room temperature and extracted with co-solvent of DCM/MeOH (10/1) (5×10 mL). The combined organic layers were washed with brine (3×10 mL) and dried over anhydrous Na₂SO₄. After filtration, the filtrate was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 mL/min; Gradient: 30% B to 60% B in 9 min; Detector: UV 254/210 nm; Retention time: 7.44 min. The fractions containing desired product were collected and concentrated under reduced pressure to afford Compound 2 ((1-(4,5-dichloro-2-hydroxybenzyl)piperidine-2,4-diyl)dimethanol) as an off-white solid (26 mg, 18%): LCMS (ESI) calculated for C₁₄H₁₉C₁₂NO₃ [M+H]⁺: 320, 322 (3:2), found 320, 322 (3:2); ¹H NMR (300 MHz, CD₃OD) δ 7.15 (s, 1H), 6.85 (s, 1H), 4.44 (d, J=14.4 Hz, 1H), 3.82 (dd, J=11.9, 4.0 Hz, 1H), 3.61 (dd, J=11.8, 3.7 Hz, 1H), 3.39 (d, J=6.1 Hz, 2H), 3.22-3.20 (m, 1H), 3.01-2.88 (m, 1H), 2.48-2.25 (m, 1H), 2.24-2.04 (m, 1H), 1.84-1.51 (m, 3H), 1.37-1.04 (m, 2H).

Step b′:

To a mixture of methyl 4-(hydroxymethyl)piperidine-2-carboxylate (71 mg, 0.41 mmol) and K₂CO₃ (0.15 g, 1.11 mmol) in DMF (3 mL) was added 1-(bromomethyl)-4,5-dichloro-2-methoxybenzene (0.10 g, 0.37 mmol) at room temperature. The reaction mixture was stirred for 3 h at 45° C. The resulting mixture was poured into water (20 mL) and extracted with EA (3×20 mL). The combined organic layers were washed with brine (2×20 mL), dried over anhydrous Na₂SO₄ and filtered. The filtrate was concentrated under reduced pressure. The residue was purified by Prep-TLC (PE/EA 2/1) to afford methyl 1-[(4,5-dichloro-2-methoxyphenyl)methyl]-4-(hydroxymethyl)piperidine-2-carboxylate as an off-white solid (89 mg, 66%): LCMS (ESI) calculated for C₁₆H₂₁C₁₂NO₄ [M+H]⁺: 362, 364 (3:2), found 362, 364 (3:2); ¹H NMR (300 MHz, CDCl₃) δ 7.51 (s, 1H), 6.92 (s, 1H), 3.77 (d, J=9.4 Hz, 6H), 3.64-3.45 (m, 4H), 3.12-3.02 (m, 1H), 2.14-1.98 (m, 2H), 1.74-1.43 (m, 5H).

Step c′:

To a stirred solution of methyl 1-(4,5-dichloro-2-methoxybenzyl)-4-(hydroxymethyl)piperidine-2-carboxylate (0.10 g, 0.29 mmol) in DCM (2 mL) was added BBr₃ (0.43 g, 1.72 mol) dropwise at room temperature under nitrogen atmosphere. The reaction mixture was stirred at room temperature for 2 h under nitrogen atmosphere. The resulting mixture was quenched with water (10 mL) and adjusted pH value to 7 with saturated aq. NaHCO₃. The aqueous layer was extracted with EA (3×20 mL). Then the combined organic layers were washed with brine (2×20 mL), dried over anhydrous Na₂SO₄ and filtered. The filtrate was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmoL/L NH₄HCO₃, Mobile Phase B: ACN; Flow rate: 20 mL/min; Gradient: 20% B to 80% B in 9 min; Detector: UV 254/210 nm; Retention time: 8.14 min. The fractions containing desired product were collected and concentrated under reduced pressure to afford Compound 1 (methyl 1-[(4,5-dichloro-2-hydroxyphenyl)methyl]-4-(hydroxymethyl)piperidine-2-carboxylate) as an off-white solid (40 mg, 39%): LCMS (ESI) calculated for C₁₅H₁₉C₁₂NO₄ [M+H]⁺: 348, 350 (3:2), found 348, 350 (3:2); ¹H NMR (400 MHz, CD₃OD) δ 7.34 (s, 1H), 6.98 (s, 1H), 4.04 (d, J=13.2 Hz, 1H), 3.82 (s, 3H), 3.77-3.65 (m, 1H), 3.54-3.33 (m, 3H), 3.22-3.20 (m, 1H), 2.46 (s, 1H), 2.16 (d, J=10.4 Hz, 1H), 1.80 (d, J=10.4 Hz, 1H), 1.70 (s, 1H), 1.47-1.33 (m, 2H).

### Example 4. Compound 3 (2-((4-amino-4-(aminomethyl)piperidin-1-yl)methyl)-4,5-dichlorophenol)

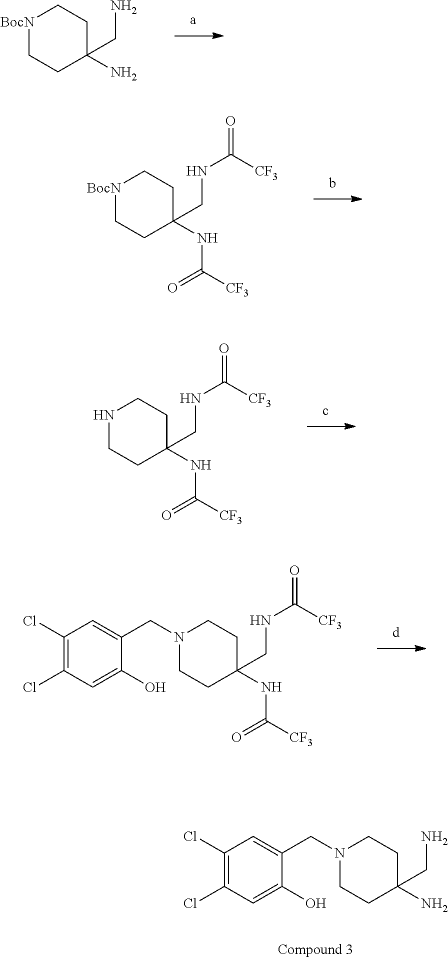 [SMILES: CC(C)(C)OC(=O)N1CCC(N)(CN)CC1 ; CC(C)(C)OC(=O)N1CCC(CNC(=O)C(F)(F)F)(NC(=O)C(F)(F)F)CC1 ; O=C(NCC1(NC(=O)C(F)(F)F)CCNCC1)C(F)(F)F ; O=C(NCC1(NC(=O)C(F)(F)F)CCN(Cc2cc(Cl)c(Cl)cc2O)CC1)C(F)(F)F ; NCC1(N)CCN(Cc2cc(Cl)c(Cl)cc2O)CC1]

Step a:

To a stirred solution of tert-butyl 4-amino-4-(aminomethyl)piperidine-1-carboxylate (0.20 g, 0.87 mmol) and Et₃N (0.44 g, 4.36 mmol) in DCM (4 mL) was added 2,2,2-trifluoroacetic anhydride (0.55 g, 2.62 mmol) at room temperature. The reaction solution was stirred for 1 h at room temperature. The reaction mixture was quenched with water (30 mL) and extracted with EA (3×30 mL). The combined organic layers were washed with brine (2×20 mL), dried over anhydrous Na₂SO₄ and filtered. The filtrate was concentrated under reduced pressure. The residue was purified by reverse phase chromatography, eluted with 40% ACN in water (plus 0.05% TFA) to afford tert-butyl 4-(2,2,2-trifluoroacetamido)-4-[(2,2,2-trifluoroacetamido)methyl]piperidine-1-carboxylate as an off-white solid (0.32 g, 78%): LCMS (ESI) calculated for C₁₅H₂₁F₆N₃O₄[M+H]⁺: 422, found 422; ¹H NMR (300 MHz, DMSO-d₆) δ 9.47 (t, J=6.3 Hz, 1H), 8.64 (s, 1H), 3.65 (d, J=13.8 Hz, 2H), 3.46 (d, J=6.2 Hz, 2H), 2.91-2.68 (m, 2H), 2.54-2.48 (m, 2H), 2.15 (d, J=13.7 Hz, 2H), 1.35 (s, 9H); ¹⁹F NMR (282 MHz, DMSO-d₆) δ −73.75.

Step b:

To a stirred solution of tert-butyl 4-(2,2,2-trifluoroacetamido)-4-[(2,2,2-trifluoroacetamido)methyl]piperidine-1-carboxylate (0.32 g, 0.76 mmol) in DCM (1 mL) was added TFA (1 mL) at room temperature. The reaction solution was stirred for 1 h at room temperature. The resulting solution was concentrated under reduced pressure to afford 2,2,2-trifluoro-N-[[4-(2,2,2-trifluoroacetamido)piperidin-4-yl]methyl]acetamide as a colorless oil (0.12 g, crude): LCMS (ESI) calculated for C₁₀H₁₃F₆N₃O₂ [M+H]⁺: 322, found 322.

Step c:

To a stirred solution of 2,2,2-trifluoro-N-[[4-(trifluoroacetamido)piperidin-4-yl]methyl]acetamide (0.12 g, 0.38 mmol) and Intermediate 1 (87 mg, 0.46 mmol) in MeOH (2 mL) were added HOAc (25 mg, 0.42 mmol) and NaBH(OAc)₃ (0.24 g, 1.14 mmol) at room temperature. After stirring for 2 h at room temperature, the resulting mixture was quenched with water (10 mL) and extracted with EA (3×30 mL). Then the combined organic layers were washed with brine (2×20 mL), dried over anhydrous Na₂SO₄ and filtered. The filtrate was concentrated under reduced pressure. The residue was purified by Prep-TLC, eluted with PE/EA (3/1) to afford N-([1-[(4,5-dichloro-2-hydroxyphenyl)methyl]-4-(trifluoroacetamido)piperidin-4-yl]methyl)-2,2,2-trifluoroacetamide as a light yellow solid (63 mg, 27%): LCMS (ESI) calculated for C₁₇H₁₇C₁₂F₆N₃O₃ [M+H]⁺: 496, 498 (3:2), found 496, 498 (3:2); ¹H NMR (300 MHz, DMSO-d₆) δ 9.45 (d, J=5.9 Hz, 1H), 8.56 (s, 1H), 7.36 (s, 1H), 6.90 (s, 1H), 3.56 (s, 2H), 3.15 (s, 2H), 2.67-2.54 (m, 2H), 2.29-2.00 (m, 4H), 1.61-1.42 (m, 2H); ¹⁹F NMR (282 MHz, DMSO-d₆) δ −73.84, 74.00.

Step d:

To a stirred solution of N-([1-[(4,5-dichloro-2-hydroxyphenyl)methyl]-4-(trifluoroacetamido)piperidin-4-yl]methyl)-2,2,2-trifluoroacetamide (63 mg, 0.13 mmol) in MeOH (2 mL) was added saturated aq. NaOH (2 mL) at room temperature. The reaction solution was stirred at room temperature for 2 h. The resulting solution was adjusted pH to 7 with aq. HCl (1 N) and concentrated under reduced pressure. The residue was purified by Prep-HPLC with the following conditions: Column: X Bridge C₁₈ OBD Prep Column 100 Å, 10 μm, 19 mm×250 mm; Mobile Phase A: water with 20 mmoL/L NH₄HCO₃, Mobile Phase B: ACN; Flow rate: 20 mL/min; Gradient: 30% B to 80% B in 9 min; Detector: UV 254/210 nm; Retention time: 7.74 min. The fractions containing desired product were collected and concentrated under reduced pressure to afford Compound 3 (2-((4-amino-4-(aminomethyl)piperidin-1-yl)methyl)-4,5-dichlorophenol) as an off-white solid (25.8 mg, 50%): LCMS (ESI) calculated for C₁₃H₁₉C₁₂N₃O [M+H]⁺: 304, 306 (3:2), found 304, 306 (3:2); ¹H NMR (300 MHz, CD₃OD) δ 7.56 (s, 1H), 7.09 (s, 1H), 4.30 (s, 2H), 3.58-3.38 (m, 4H), 3.12-2.96 (m, 2H), 2.15-1.97 (m, 4H); ¹⁹F NMR (376 MHz, CD₃OD) δ −77.20.

### Example 5. Compound 4 (2-[2-amino-1-[4-(hydroxymethyl)piperidin-1-yl]ethyl]-4,5-dichlorophenol)

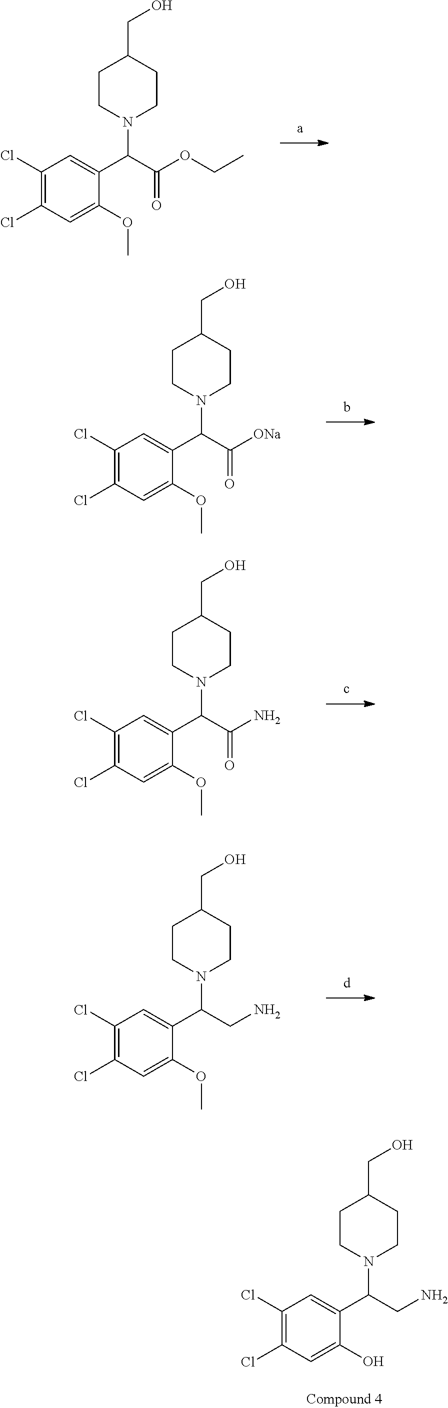

Step a:

To a stirred solution of ethyl 2-(4,5-dichloro-2-methoxyphenyl)-2-[4-(hydroxymethyl)piperidin-1-yl]acetate (Example 15, Step D) (0.15 g, 0.40 mmol) in MeOH (1 mL) and H₂O (0.2 mL) was added NaOH (32 mg, 0.80 mmol) at room temperature. The reaction solution was stirred at room temperature for 16 h. The resulting solution was concentrated under reduced pressure to afford sodium 2-(4,5-dichloro-2-methoxyphenyl)-2-[4-(hydroxymethyl)piperidin-1-yl] acetate as a light yellow solid (0.10 g, crude), which was used for next step directly without further purification: LCMS (ESI) calculated for C₁₅H₁₉C₁₂NO₄ [M+H]⁺ 348, 350 (3:2), found 348, 350 (3:2).

Step b:

To a stirred solution of sodium 2-(4,5-dichloro-2-methoxyphenyl)-2-[4-(hydroxymethyl)piperidin-1-yl] acetate (0.10 g, 0.29 mmol) in DMF (3 mL) were added HATU (49 mg, 0.57 mmol), NH₄Cl (31 mg, 0.57 mmol) and Et₃N (58 mg, 0.57 mmol) at room temperature. The reaction solution was stirred at room temperature for 16 h. The resulting solution was quenched with water (20 mL) and extracted with EA (3×30 mL). The combined organic layers were washed with brine (2×10 mL) and dried over anhydrous Na₂SO₄. After filtration, the filtrate was concentrated under reduced pressure. The residue was purified by reverse phase chromatography, eluted with 33% ACN in water (plus 0.05% TFA) to afford 2-(4,5-dichloro-2-methoxyphenyl)-2-[4-(hydroxymethyl)piperidin-1-yl]acetamide as an off-white solid (50 mg, 45%): LCMS (ESI) calculated for C₁₅H₂₀Cl₂N₂O₃ [M+H]⁺ 347, 349 (3:2), found 347, 349 (3:2).

Step c:

To a stirred solution of 2-(4,5-dichloro-2-methoxyphenyl)-2-[4-(hydroxymethyl)piperidin-1-yl]acetamide (0.13 g, 0.37 mmol) in THE (2 mL) was added BH₃.THF (0.75 mL, 0.75 mmol, 1 M in THF) at 0° C. under argon atmosphere. The reaction solution was allowed to warm to 70° C. and stirred for 3 h. After cooling to room temperature, the resulting solution was quenched with water (1 mL) at room temperature and concentrated under reduced pressure. The residue was purified by reverse phase chromatography, eluted with 37% ACN in water (plus 0.05% TFA) to afford [1-[2-amino-1-(4,5-dichloro-2-methoxyphenyl)ethyl]piperidin-4-yl]methanol as a colorless oil (70 mg, 47%): LCMS (ESI) calculated for C₁₅H₂₂Cl₂N₂O₂ [M+H]⁺: 333, 335 (3:2), found 333, 335 (3:2); ¹H NMR (300 MHz, CD₃OD) δ 7.65 (s, 1H), 7.41 (s, 1H), 3.95 (s, 3H), 3.87-3.74 (m, 1H), 3.73-3.50 (m, 2H), 3.42 (d, J=5.2 Hz, 2H), 2.93-2.75 (m, 1H), 2.71-2.65 (m, 1H), 1.98-1.87 (m, 2H), 1.74-1.43 (m, 3H), 1.33-1.18 (m, 1H), 1.01-0.79 (m, 1H).

Step d:

To a stirred solution of [1-[2-amino-1-(4,5-dichloro-2-methoxyphenyl)ethyl]piperidin-4-yl]methanol (80 mg, 0.24 mmol) in DCM (3 mL) was added BBr₃ (0.36 g, 1.44 mmol) at room temperature. The reaction mixture was stirred at room temperature for 16 h. The resulting mixture was quenched with water (1 mL) at room temperature and concentrated under reduced pressure. The residue was purified by Prep-HPLC with following conditions: Column: XBridge C₁₈ OBD Prep Column 100 Å, 10 μm, 19 mm×250 mm; Mobile Phase A: water with 20 mmoL/L NH₄HCO₃, Mobile Phase B: ACN; Flow rate: 20 mL/min; Gradient: 25% B to 65% B in 9 min; Detector: UV 254/210 nm; Retention time: 6.67 min. The fractions containing desired product were collected and concentrated under reduced pressure to afford Compound 4 (2-[2-amino-1-[4-(hydroxymethyl)piperidin-1-yl]ethyl]-4,5-dichlorophenol) as an off-white solid (14.1 mg, 17%): LCMS (ESI) calculated for C₁₄H₂₀Cl₂N₂O₂ [M+H]⁺: 319, 321 (3:2), found 319, 321 (3:2); ¹H NMR (400 MHz, CD₃OD) δ 7.23 (s, 1H), 6.92 (s, 1H), 3.71-3.66 (m, 1H), 3.45-3.38 (m, 2H), 3.24-3.14 (m, 2H), 3.07-2.92 (m, 2H), 2.23 (t, J=11.5 Hz, 1H), 2.14-2.03 (m, 1H), 1.88-1.72 (m, 2H), 1.55-1.48 (m, 1H), 1.38-1.23 (m, 2H).

### Example 6. Compound 5 (2-[[4-(aminomethyl)-4-(hydroxymethyl)piperidin-1-yl]methyl]-4,5-dichlorophenol)

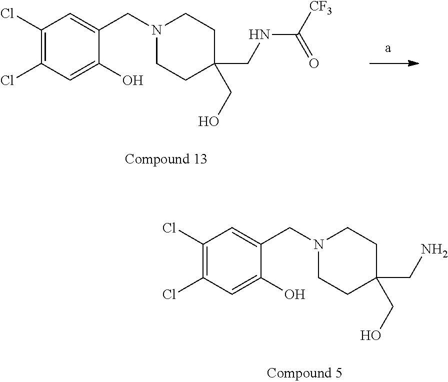 [SMILES: O=C(NCC1(CO)CCN(Cc2cc(Cl)c(Cl)cc2O)CC1)C(F)(F)F ; NCC1(CO)CCN(Cc2cc(Cl)c(Cl)cc2O)CC1]

Step a:

To a stirred solution of N-([1-[(4,5-dichloro-2-hydroxyphenyl)methyl]-4-(hydroxymethyl)piperidin-4-yl]methyl)-2,2,2-trifluoroacetamide) (Compound 13, Example 13) (66 mg, 0.16 mmol) in MeOH (2 mL) was added saturated aq. NaOH (0.5 mL) at room temperature. The reaction solution was stirred at room temperature for 2 h. The resulting solution was adjusted pH to 7 with aq. HCl (1 N) and concentrated under reduced pressure. The residue was purified by Prep-HPLC with following conditions: Column: XBridge C₁₈ OBD Prep Column, 100 Å, 10 μm, 19 mm×250 mm; Mobile Phase A: water with 20 mmol/L NH₄HCO₃, Mobile Phase B: ACN; Flow rate: 20 mL/min; Gradient: 30% B to 80% B in 9 min; Detector: UV 254/210 nm; Retention time: 8.44 min. The fractions containing desired product were collected and concentrated under reduced pressure to afford Compound 5 (2-[[4-(aminomethyl)-4-(hydroxymethyl)piperidin-1-yl]methyl]-4,5-dichlorophenol) as a light yellow solid (38 mg, 67%): LCMS (ESI) calculated for C₁₄H₂Cl₂N₂O₂ [M+H]⁺: 319, 321 (3:2), found 319, 321 (3:2); ¹H NMR (300 MHz, CD₃OD) δ 7.19 (s, 1H), 6.86 (s, 1H), 3.71 (s, 2H), 3.53 (s, 2H), 2.72 (s, 2H), 2.65-2.56 (m, 4H), 1.59-1.50 (m, 4H).

### Example 7. Compound 6 (4,5-dichloro-2-[[4-(hydroxymethyl)-4-[(pyrrolidin-1-yl)carbonyl]piperidin-1-yl]methyl]phenol)

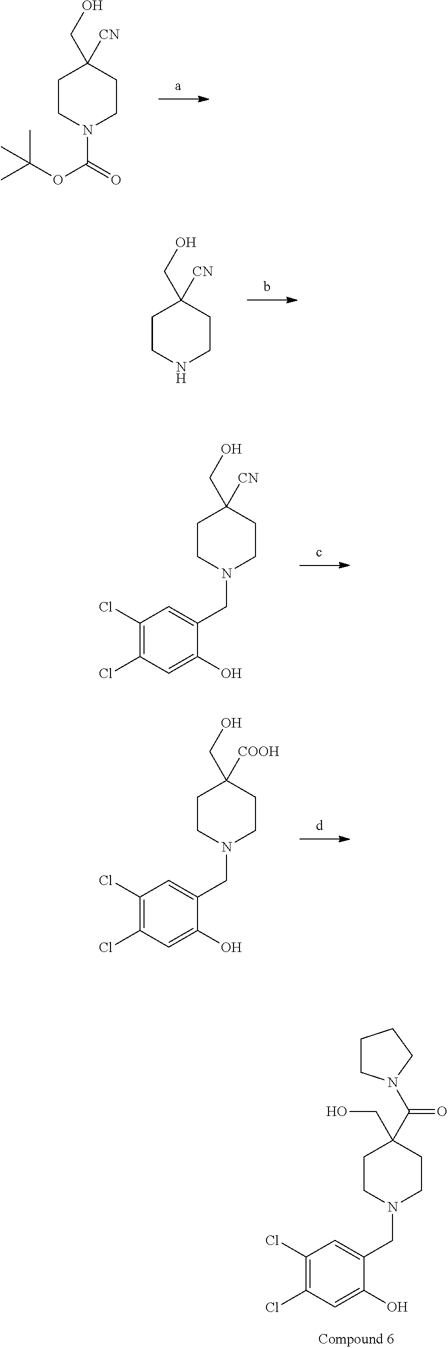 [SMILES: CC(C)(C)OC(=O)N1CCC(C#N)(CO)CC1 ; N#CC1(CO)CCNCC1 ; N#CC1(CO)CCN(Cc2cc(Cl)c(Cl)cc2O)CC1 ; O=C(O)C1(CO)CCN(Cc2cc(Cl)c(Cl)cc2O)CC1 ; O=C(N1CCCC1)C1(CO)CCN(Cc2cc(Cl)c(Cl)cc2O)CC1]

Step a:

To a solution of tert-butyl 4-cyano-4-(hydroxymethyl)piperidine-1-carboxylate (Example 25, Step a) (0.20 g, 0.83 mmol) in DCM (2 mL) was added TFA (2 mL) at room temperature. After stirring for 1 h at room temperature, the resulting solution was concentrated under reduced pressure. The residue was diluted with water (10 mL), and adjusted pH value to 7 with saturated aq. K₂CO₃. The aqueous layer was extracted with DCM (10×20 mL). The combined organic layers were dried over anhydrous Na₂SO₄ and filtered. The filtrate was concentrated under reduced pressure to 4-(hydroxymethyl)piperidine-4-carbonitrile as a yellow oil (0.10 g, crude), which was used in next step without further purification: LCMS (ESI) calculated for C₇H₁₂N₂O [M+H]⁺: 141, found 141.

Step b:

To a stirred solution of 4-(hydroxymethyl)piperidine-4-carbonitrile (0.20 g, 1.43 mmol) and Intermediate 1 (0.27 g, 1.43 mmol) in MeOH (3.5 mL) were added HOAc (85 mg, 1.43 mmol) and NaBH(OAc)₃ (0.90 g, 4.28 mmol) at room temperature under nitrogen atmosphere. The resulting mixture was stirred at room temperature for 1 h. The reaction mixture was quenched with water (1 mL) and concentrated under reduced pressure. The residue was purified by silica gel column chromatography, eluted with PE/EA (2/3) to afford 1-[(4,5-dichloro-2-hydroxyphenyl)methyl]-4-(hydroxymethyl)piperidine-4-carbonitrile as a yellow solid (0.20 g, 60%): LCMS (ESI) calculated for C₁₄H₁₆Cl₂N₂O₂ [M+H]⁺: 315, 317 (3:2), found 315, 317 (3:2); ¹H NMR (300 MHz, CDCl₃) δ 7.07 (s, 1H), 6.89 (s, 1H), 3.72 (s, 2H), 3.60 (s, 2H), 3.07-2.97 (m, 2H), 2.54-2.39 (m, 3H), 2.08-1.96 (m, 2H), 1.72-1.56 (m, 2H).

Step c:

A solution of 1-[(4,5-dichloro-2-hydroxyphenyl)methyl]-4-(hydroxymethyl)piperidine-4-carbonitrile (0.15 g, 0.48 mmol) in aq. HCl (3 mL, 12N) was stirred at 80° C. for 2 h. After cooling to room temperature, the resulting solution was concentrated under reduced pressure to afford 1-[(4,5-dichloro-2-hydroxyphenyl)methyl]-4-(hydroxymethyl)piperidine-4-carboxylic acid as a light yellow solid (0.12 g, crude), which was used in next step without further purification: LCMS (ESI) calculated for C₁₄H₁₇Cl₂NO₄ [M+H]⁺: 334, 336 (3:2), found 334, 336 (3:2).

Step d:

To a stirred solution of 1-[(4,5-dichloro-2-hydroxyphenyl)methyl]-4-(hydroxymethyl)piperidine-4-carboxylic acid (0.12 g, 0.36 mmol) in DMF (3 mL) was added pyrrolidine (51 mg, 0.72 mmol), HATU (0.27 g, 0.72 mmol) and Et₃N (0.11 g, 1.08 mmol) at room temperature. The reaction solution was stirred at room temperature for 16 h. The resulting solution was quenched with water (3 mL) and concentrated under reduced pressure. The residue was purified by Prep-HPLC with following conditions: Column: XBridge C₁₈ OBD Prep Column 100 Å, 10 μm, 19 mm×250 mm; Mobile Phase A: water with 20 mmoL/L NH₄HCO₃, Mobile Phase B: ACN; Flow rate: 20 mL/min; Gradient: 30% B to 80% B in 9 min; Detector: UV 254/210 nm; Retention time: 8.28 min. The fractions containing desired product were collected and concentrated under reduced pressure to afford Compound 6 (4,5-dichloro-2-[[4-(hydroxymethyl)-4-[(pyrrolidin-1-yl)carbonyl]piperidin-1-yl]methyl]phenol) as an off-white solid (24.1 mg, 16%): LCMS (ESI) calculated for C₁₈H₂₄Cl₂N₂O₃ [M+H]⁺: 387, 389 (3:2), found 387, 389 (3:2); ¹H NMR (400 MHz, CD₃OD) δ 7.18 (s, 1H), 6.86 (s, 1H), 3.89-3.42 (m, 8H), 2.82-2.78 (m, 2H), 2.40-2.32 (m, 4H), 2.04-1.88 (m, 4H), 1.66-1.57 (t, J=11.5 Hz, 2H).

### Example 8. Compound 7 (N-([1-[(4,5-dichloro-2-hydroxyphenyl)methyl]-4-(hydroxymethyl)piperidin-4-yl]methyl)prop-2-enamide)

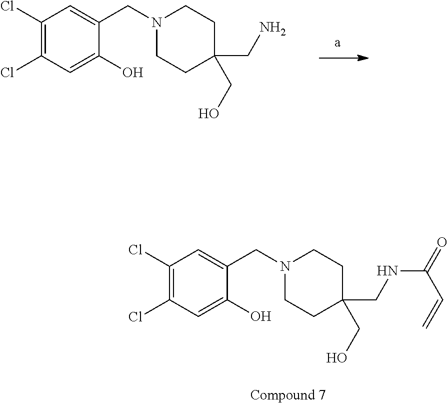 [SMILES: NCC1(CO)CCN(Cc2cc(Cl)c(Cl)cc2O)CC1 ; C=CC(=O)NCC1(CO)CCN(Cc2cc(Cl)c(Cl)cc2O)CC1]

Step a:

To a stirred solution of 2-[[4-(aminomethyl)-4-(hydroxymethyl)piperidin-1-yl]methyl]-4,5-dichlorophenol (38 mg, 0.12 mmol) and Et₃N (18 mg, 0.18 mmol) in DCM (2 mL) was added prop-2-enoyl chloride (11 mg, 0.12 mmol) at room temperature under nitrogen atmosphere. The reaction solution was stirred at room temperature for 1.5 h. The resulting solution was quenched with water (1 mL) and concentrated under reduced pressure. The residue was purified by Prep-HPLC with the following conditions: Column: X Bridge C₁₈ OBD Prep Column 100 Å, 10 μm, 19 mm×250 mm; Mobile Phase A: water with 20 mmoL/L NH₄HCO₃, Mobile Phase B: ACN; Flow rate: 20 mL/min; Gradient: 30% B to 80% B in 9 min; Detector: UV 254/210 nm; Retention time: 7.84 min. The fractions containing desired product were collected and concentrated under reduced pressure to afford Compound 7 (N-([1-[(4,5-dichloro-2-hydroxyphenyl)methyl]-4-(hydroxymethyl)piperidin-4-yl]methyl)prop-2-enamide) as an off-white solid (8.6 mg, 19%): LCMS (ESI) calculated for C₁₇H₂₂Cl₂N₂O₃ [M+H]⁺: 373, 375 (3:2), found 373, 375 (3:2); ¹H NMR (300 MHz, CD₃OD) δ 7.19 (s, 1H), 6.86 (s, 1H), 6.33-6.24 (m, 2H), 5.71-5.63 (m, 1H), 3.73 (s, 2H), 3.32 (s, 4H), 2.62-2.58 (m, 4H), 1.60-1.49 (m, 4H).

### Example 9. Compound 8 (N-([1-[(4,5-dichloro-2-hydroxyphenyl)methyl]-4-hydroxypiperidin-4-yl]methyl)acetamide)

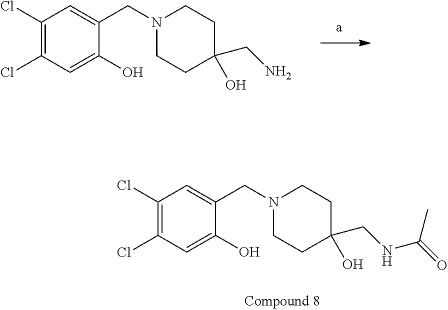 [SMILES: NCC1(O)CCN(Cc2cc(Cl)c(Cl)cc2O)CC1 ; CC(=O)NCC1(O)CCN(Cc2cc(Cl)c(Cl)cc2O)CC1]

Step a:

To a mixture of 4-(aminomethyl)-1-[(4,5-dichloro-2-hydroxyphenyl)methyl]piperidin-4-ol (0.19 g, 0.62 mmol) and NaOH (49 mg, 1.24 mmol) in EtOH (4 mL) was added acetic anhydride (65 mg, 0.63 mmol) at room temperature. The reaction mixture was stirred for 3 h at room temperature. The resulting mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 mL/min; Gradient: 35% B to 38% B in 9 min; Detector: UV 254/210 nm; Retention time: 7.85 min. The fractions containing desired product were collected and concentrated under reduced pressure to afford Compound 8 (N-([1-[(4,5-dichloro-2-hydroxyphenyl)methyl]-4-hydroxypiperidin-4-yl]methyl)acetamide) as an off-white solid (60 mg, 27%): LCMS (ESI) calculated for C₁₅H₂₀Cl₂N₂O₃ [M+H]⁺: 347, 349 (3:2), found 347, 349 (3:2); ¹H NMR (400 MHz, CD₃OD) δ 7.22 (s, 1H), 6.88 (s, 1H), 3.74 (s, 2H), 3.24 (s, 2H), 2.74 (d, J=11.7 Hz, 2H), 2.58 (t, J=10.8 Hz, 2H), 2.00 (s, 3H), 1.71-157 (m, 4H).

### Example 10. Compound 9 (4-(aminomethyl)-1-[(4,5-dichloro-2-hydroxyphenyl)methyl]piperidin-4-ol trifluoroacetic acid)

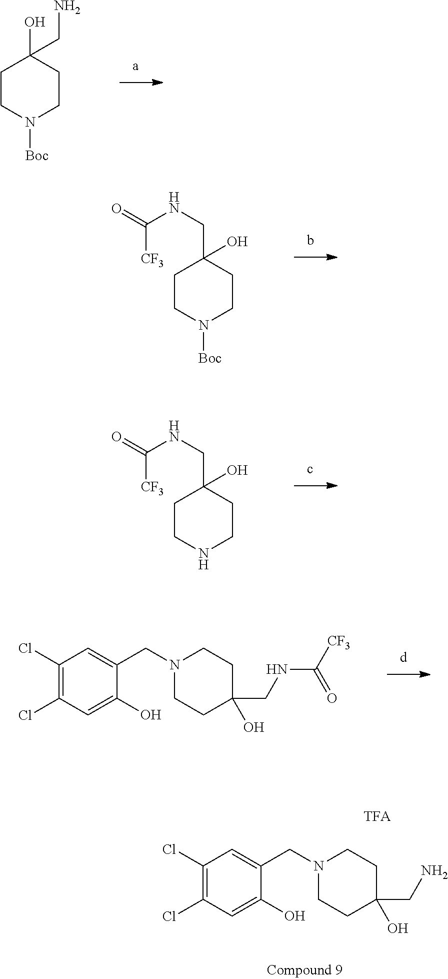 [SMILES: CC(C)(C)OC(=O)N1CCC(O)(CN)CC1 ; CC(C)(C)OC(=O)N1CCC(O)(CNC(=O)C(F)(F)F)CC1 ; O=C(NCC1(O)CCNCC1)C(F)(F)F ; O=C(NCC1(O)CCN(Cc2cc(Cl)c(Cl)cc2O)CC1)C(F)(F)F ; NCC1(O)CCN(Cc2cc(Cl)c(Cl)cc2O)CC1]

Step a:

To a solution of tert-butyl 4-(aminomethyl)-4-hydroxypiperidine-1-carboxylate (2.00 g, 8.68 mmol) in DCM (20 mL) were added trifluoroacetyl 2,2,2-trifluoroacetate (1.83 g, 8.71 mmol) and Et₃N (1.32 g, 13.04 mmol) dropwise at room temperature under nitrogen atmosphere. The reaction solution was stirred for 3 h at room temperature under nitrogen atmosphere. The resulting solution was concentrated under reduced pressure. The residue was diluted with DCM (50 mL) and washed with saturated aq. NaHCO₃ (2×50 mL). The organic phase was dried over anhydrous Na₂SO₄ and filtered. The filtrate was concentrated under reduced pressure to afford tert-butyl 4-hydroxy-4-[(trifluoroacetamido)methyl]piperidine-1-carboxylate as a light yellow solid (2.65 g, crude): LCMS (ESI) calculated for C₁₃H₂₁F₃N₂O₄ [M+H]⁺: 327, found 327; ¹H NMR (300 MHz, DMSO-d₆) δ 9.26 (s, 1H), 4.67 (s, 1H), 3.70-3.61 (m, 2H), 3.18 (d, J=6.2 Hz, 2H), 3.03 (s, 2H), 1.38 (s, 13H).

Step b:

A solution of tert-butyl 4-hydroxy-4-[(trifluoroacetamido)methyl]piperidine-1-carboxylate (1.30 g, 3.98 mmol) in DCM (6 mL) and TFA (3 mL) was stirred for 1 h at room temperature. The resulting solution was diluted with water (20 mL) at room temperature and basified to pH 7-8 with saturated aq. NaHCO₃. The resulting solution was concentrated under reduced pressure to afford the crude product. The crude product was triturated in MeOH (50 mL). The resulting mixture was filtered and the filter cake was washed with MeOH (3×10 mL). The filtrate was concentrated under reduced pressure to afford 2,2,2-trifluoro-N-[(4-hydroxypiperidin-4-yl)methyl]acetamide as a colorless oil (1.40 g, crude): LCMS (ESI) calculated for C₈H₁₃F₃N₂O₂ [M+H]⁺: 227, found 227.

Step c:

To a solution of 2,2,2-trifluoro-N-[(4-hydroxypiperidin-4-yl)methyl]acetamide (0.27 g, 1.19 mmol), HOAc (72 mg, 1.20 mmol) and Intermediate 1 (0.23 g, 1.21 mmol) in MeOH (10 mL) was added NaBH(OAc)₃ (0.76 g, 3.52 mmol) at room temperature under nitrogen atmosphere. The reaction solution was stirred at room temperature for 1 h under nitrogen atmosphere. The resulting solution was quenched with water (2 mL) and concentrated under reduced pressure. The residue was purified by silica gel column chromatography, eluted with DCM/MeOH (20/1) to afford N-([1-[(4,5-dichloro-2-hydroxyphenyl)methyl]-4-hydroxypiperidin-4-yl]methyl)-2,2,2-trifluoroacetamide as a yellow semi-solid (0.10 g, 22%): LCMS (ESI) calculated for C₁₅H₁₇Cl₂F₃N₂O₃ [M+H]⁺: 401, 403 (3:2), found 401, 403 (3:2); ¹H NMR (300 MHz, CD₃OD) δ 7.37 (s, 1H), 6.96 (s, 1H), 3.99 (s, 2H), 3.28 (d, J=1.6 Hz, 2H), 3.13-2.87 (m, 4H), 1.89-1.61 (m, 4H).

Step d:

To a solution of N-([1-[(4,5-dichloro-2-hydroxyphenyl)methyl]-4-hydroxypiperidin-4-yl]methyl)-2,2,2-trifluoroacetamide (0.10 g, 0.25 mmol) in EtOH (2 mL) and water (1 mL) was added NaOH (0.10 g, 2.50 mmol) at room temperature. After stirring for 2 h at room temperature, the resulting solution was concentrated under reduced pressure. The residue was purified by Prep-HPLC with the following conditions: Column: XBridge C₁₈ OBD Prep Column 100 Å, 10 μm, 19 mm×250 mm; Mobile Phase A: water (plus 0.05% TFA), Mobile Phase B: ACN; Flow rate: 20 mL/min; Gradient: 20% B to 60% B in 8 min; Detector: 210/254 nm; Retention time: 6 min. The fractions containing desired product were collected and concentrated under reduced pressure to afford Compound 9 (4-(aminomethyl)-1-[(4,5-dichloro-2-hydroxyphenyl)methyl]piperidin-4-ol trifluoroacetic acid) as a purple solid (17 mg, 16%): LCMS (ESI) calculated for C₁₃H₁₈Cl₂N₂O₂ [M+H]⁺: 305, 307 (3:2), found 305, 307 (3:2); ¹H NMR (400 MHz, CD₃OD) δ 7.58 (s, 1H), 7.10 (s, 1H), 4.35 (s, 2H), 3.42 (d, J=15.6 Hz, 4H), 3.00 (s, 2H), 1.93 (s, 4H); ¹⁹F NMR (376 MHz, CD₃OD) δ −76.98.

### Example 11. Compound 11 (4,5-dichloro-2-(((2R,4R)-rel-4-(hydroxymethyl)-2-phenylpiperidin-1-yl)methyl)phenol) and Compound 10 (4,5-dichloro-2-(((2S,4R)-rel-4-(hydroxymethyl)-2-phenylpiperidin-1-yl)methyl)phenol)

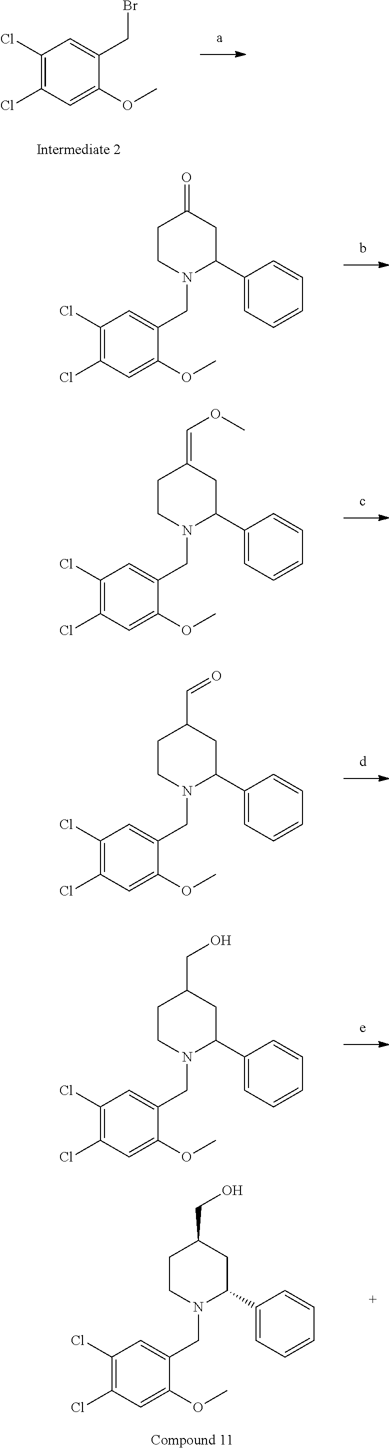

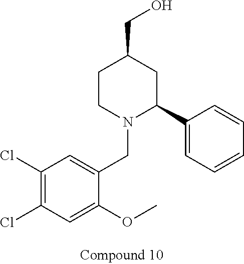 [SMILES: COc1cc(Cl)c(Cl)cc1CN1CC[C@@H](CO)C[C@H]1c1ccccc1]

Step a:

To a mixture of 2-phenylpiperidin-4-one (0.49 g, 2.78 mmol) and K₂CO₃ (0.51 g, 3.70 mmol) in DMF (8 mL) was added Intermediate 2 (0.50 g, 1.85 mmol) at room temperature. The reaction mixture was allowed to warm at 40° C. and stirred for 16 h. The resulting mixture was diluted with water (50 mL) and extracted with EA (2×50 mL). The combined organic layers were washed with brine (2×50 mL), dried over anhydrous Na₂SO₄ and filtered. The filtrate was concentrated under reduced pressure. The residue was purified by Prep-TLC, eluted with PE/EA (4/1) to afford 1-[(4,5-dichloro-2-methoxyphenyl)methyl]-2-phenylpiperidin-4-one as a colorless oil (0.40 g, 59%): LCMS (ESI) calculated for C₁₉H₁₉Cl₂NO₂ [M+H]⁺: 364, 366 (3:2), found 364, 366 (3:2); ¹H NMR (300 MHz, CDCl₃) δ 7.54 (s, 1H), 7.45-7.29 (m, 5H), 6.87 (s, 1H), 3.75 (s, 3H), 3.65 (dd, J=10.9, 3.8 Hz, 1H), 3.52 (d, J=14.9 Hz, 1H), 3.20 (d, J=14.8 Hz, 2H), 2.78-2.61 (m, 2H), 2.55 (d, J=14.5 Hz, 1H), 2.46-2.31 (m, 2H).

Step b:

To a mixture of methoxymethyl triphenylphosphonium chloride (1.08 g, 3.29 mmol) in THE (15 mL, 185.14 mmol) was added t-BuOK (0.37 g, 3.29 mmol) at room temperature under nitrogen atmosphere. The resulting mixture was stirred for 30 min at room temperature under nitrogen atmosphere. Then a solution of 1-[(4,5-dichloro-2-methoxyphenyl)methyl]-2-phenylpiperidin-4-one (0.40 g, 1.10 mmol) in THE (2 mL) was added at room temperature. The resulting mixture was stirred for 2 h at room temperature. The reaction was quenched with water (20 mL) and exacted with EA (3×50 mL). The combined organic layers were washed with brine (2×50 mL) and dried over anhydrous Na₂SO₄. After filtration, the filtrate was concentrated under reduced pressure. The residue was purified by Prep-TLC, eluted with PE/EA (5/1) to afford (4E)-1-[(4,5-dichloro-2-methoxyphenyl)methyl]-4-(methoxymethylidene)-2-phenylpiperidine as an off-white solid (0.40 g, 92%): LCMS (ESI) calculated for C₂₁H₂₃Cl₂NO₂ [M+H]⁺: 392, 394 (3:2), found 392, 394 (3:2).

Step c:

To a solution (4E)-1-[(4,5-dichloro-2-methoxyphenyl)methyl]-4-(methoxymethylidene)-2-phenylpiperidine (0.40 g, 1.02 mmol) in THF (4 mL) was added aq. HCl (1 mL, 6 N) at room temperature. The reaction mixture was stirred for 4 h at room temperature. The resulting mixture was neutralized to pH 7 with saturated aq. NaHCO₃ and extracted with EA (3×50 mL). The combined organic layers were washed with brine (2×50 mL) and dried over anhydrous Na₂SO₄. After filtration, the filtrate was concentrated under reduced pressure to afford 1-[(4,5-dichloro-2-methoxyphenyl)methyl]-2-phenylpiperidine-4-carbaldehyde as yellow oil (0.35 g, crude), which was used in the next step directly without further purification: LCMS (ESI) calculated for C₂₀H₂₁Cl₂NO₂ [M+H]⁺: 378, 380 (3:2), found 378, 380 (3:2).

Step d:

To a solution of 1-[(4,5-dichloro-2-methoxyphenyl)methyl]-2-phenylpiperidine-4-carbaldehyde (0.35 g, 0.93 mmol) in MeOH (2 mL) in THE (5 mL) was added NaBH₄ (70 mg, 1.85 mmol) at room temperature under nitrogen atmosphere. The reaction mixture was stirred for 1 h at room temperature under nitrogen atmosphere. The resulting mixture was quenched with water (30 mL) and extracted with EA (3×80 mL). The combined organic layers were washed with brine (2×20 mL) and dried over anhydrous Na₂SO₄. After filtration, the filtrate was concentrated under reduced pressure to afford (1-(4,5-dichloro-2-methoxybenzyl)-2-phenylpiperidin-4-yl)methanol as a yellow oil (0.28 g, crude), which was used in the next step directly without further purification: LCMS (ESI) calculated for C₂₀H₂₃Cl₂NO₂ [M+H]⁺: 380, 382 (3:2), found 380, 382 (3:2).

Step e:

To a solution of [1-[(4,5-dichloro-2-methoxyphenyl)methyl]-2-phenylpiperidin-4-yl]methanol (0.19 g, 0.50 mmol) in DCM (1 mL) was added BBr₃ (1.00 g, 4.00 mmol) at room temperature. The resulting mixture was stirred for 3 h at room temperature. The reaction mixture was quenched with water (10 mL) and neutralized to pH>7 with saturated aq. NaHCO₃. The resulting mixture was concentrated under reduced pressure. The residue was purified by Prep-HPLC with the following conditions: Column: Xbridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 ml/min; Gradient: 20% B to 70% B in 9 min; Detector: UV 254/210 nm; Retention time: 7.44 min and 7.68 min.

The faster-eluting isomer was obtained as Compound 11 (4,5-dichloro-2-(((2R,4R)-rel-4-(hydroxymethyl)-2-phenylpiperidin-1-yl)methyl)phenol) as an off-white solid (3.0 mg, 2%): LCMS (ESI) calculated for C₁₉H₂₁Cl₂NO₂ [M+H]⁺: 366, 368 (3:2), found 366, 368 (3:2); ¹H NMR (300 MHz, DMSO-d₆) δ 7.39-7.31 (m, 5H), 7.26-7.21 (m, 1H), 6.90 (s, 1H), 4.53 (s, 1H), 3.55-3.32 (m, 4H), 3.21-3.16 (m, 1H), 2.73-2.65 (m, 1H), 2.30-2.26 (m, 1H), 1.91-1.76 (m, 5H).

The slower-eluting isomer was obtained as Compound 10 (4,5-dichloro-2-(((2S,4R)-rel-4-(hydroxymethyl)-2-phenylpiperidin-1-yl)methyl)phenol) as an off-white solid (16.9 mg, 9%): LCMS (ESI) calculated for C₁₉H₂₁Cl₂NO₂ [M+H]⁺: 366, 368 (3:2), found 366, 368 (3:2); ¹H NMR (300 MHz, DMSO-d₆) δ 7.39-7.31 (m, 5H), 7.26-7.21 (m, 1H), 6.90 (s, 1H), 4.46 (s, 1H), 3.46 (d, J=15.0 Hz, 1H), 3.41-3.20 (m, 3H), 3.10 (d, J=15.0 Hz, 1H), 2.95 (d, J=11.4 Hz, 1H), 2.13-2.01 (m, 1H), 1.80-1.50 (m, 3H), 1.38-1.20 (m, 2H).

### Example 12. Compound 12 (N-[1-[(4,5-dichloro-2-hydroxyphenyl)methyl]-4-(hydroxymethyl)piperidin-4-yl]acetamide)

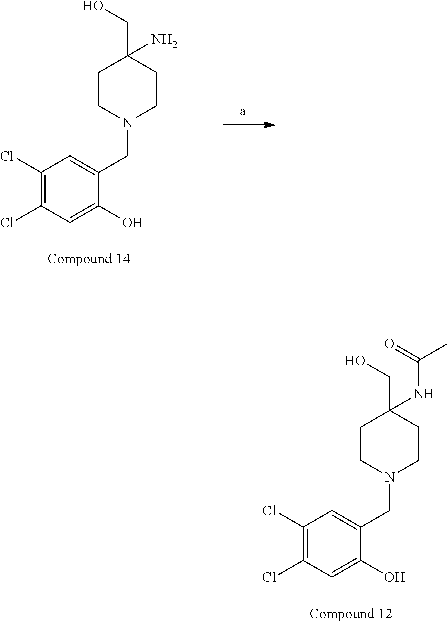 [SMILES: NC1(CO)CCN(Cc2cc(Cl)c(Cl)cc2O)CC1 ; CC(=O)NC1(CO)CCN(Cc2cc(Cl)c(Cl)cc2O)CC1]

Step a:

To a stirred solution of 2-((4-amino-4-(hydroxymethyl)piperidin-1-yl)methyl)-4,5-dichlorophenol (Compound 14, Example 14) (0.12 g, 0.30 mmol) in DCM (5 mL) was added Ac₂O (91 mg, 0.89 mmol) at room temperature. The reaction solution was stirred for 3 h at room temperature. Then NaOH (0.10 g, 2.50 mmol) and H₂O (1 mL) were added to reaction solution. The resulting mixture was stirred for additional 3 h at room temperature. The mixture was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 mL/min; Gradient: 40% B to 50% B in 16 min; Detector: UV 254/210 nm; Retention time: 9.65 min. The fraction containing desired product were collected and concentrated under reduced pressure to afford Compound 12 (N-[1-[(4,5-dichloro-2-hydroxyphenyl)methyl]-4-(hydroxymethyl)piperidin-4-yl]acetamide) as an off-white solid (49.1 mg, 48%): LCMS (ESI) calculated for C₁₅H₂₀Cl₂N₂O₃ [M+H]⁺: 347, 349 (3:2), found 347, 349 (3:2); ¹H NMR (300 MHz, CD₃OD) δ 7.19 (s, 1H), 6.86 (s, 1H), 3.67 (d, J=9.4 Hz, 4H), 2.74 (d, J=12.0 Hz, 2H), 2.39 (t, J=11.3 Hz, 2H), 2.20 (d, J=14.2 Hz, 2H), 1.96 (s, 3H), 1.73-1.57 (m, 2H).

### Example 13. Compound 13 (N-([1-[(4,5-dichloro-2-hydroxyphenyl)methyl]-4-(hydroxymethyl)piperidin-4-yl]methyl)-2,2,2-trifluoroacetamide)

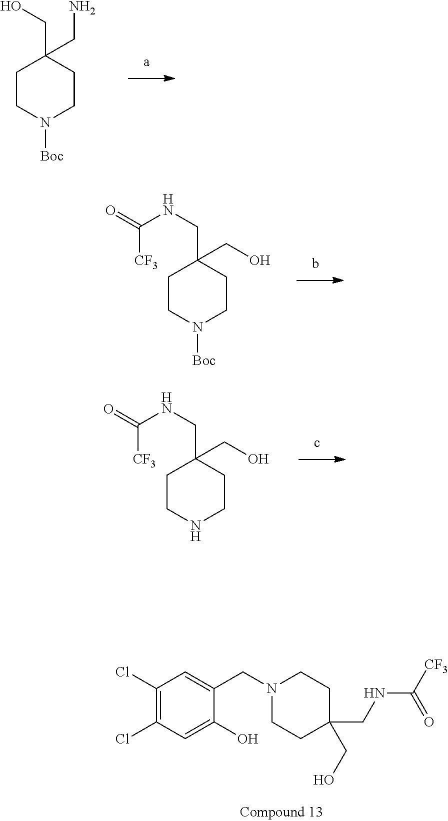 [SMILES: CC(C)(C)OC(=O)N1CCC(CN)(CO)CC1 ; CC(C)(C)OC(=O)N1CCC(CO)(CNC(=O)C(F)(F)F)CC1 ; O=C(NCC1(CO)CCNCC1)C(F)(F)F ; O=C(NCC1(CO)CCN(Cc2cc(Cl)c(Cl)cc2O)CC1)C(F)(F)F]

Step a:

To a stirred solution of tert-butyl 4-(aminomethyl)-4-(hydroxymethyl)piperidine-1-carboxylate (0.20 g, 0.82 mmol) and 2,2,2-trifluoroacetic anhydride (0.17 g, 0.82 mmol) in DCM (2 mL) was added Et₃N (0.25 g, 2.46 mmol) at room temperature. The resulting solution was stirred at room temperature for 1 h. The resulting mixture was concentrated under reduced pressure to afford tert-butyl 4-(hydroxymethyl)-4-((2,2,2-trifluoroacetamido)methyl)piperidine-1-carboxylate as a yellow oil (0.2 g, crude), which was directly used in the next step without further purification: LCMS (ESI) calculated for C₁₄H₂₃F₃N₂O₄ [M+H]⁺: 341, found 341.

Step b:

To a stirred solution of tert-butyl 4-(hydroxymethyl)-4-((2,2,2-trifluoroacetamido)methyl)piperidine-1-carboxylate (0.20 g, 0.58 mmol) in DCM (1 mL) was added TFA (1 mL) at room temperature. The resulting solution was stirred at room temperature for 1 h. The resulting mixture was concentrated under reduced pressure to afford 2,2,2-trifluoro-N-((4-(hydroxymethyl)piperidin-4-yl)methyl)acetamide as a yellow oil (0.2 g, crude), which was directly used in the next step without further purification: LCMS (ESI) calculated for C₉H₁₅F₃N₂O₂ [M+H]⁺: 241, found 241.

Step c:

To a stirred solution of 2,2,2-trifluoro-N-[[4-(hydroxymethyl)piperidin-4-yl]methyl]acetamide (0.11 g, 0.45 mmol) and Intermediate 1 (86 mg, 0.45 mmol) in MeOH (1 mL) was added HOAc (3 mg, 0.04 mmol) at room temperature. The resulting solution was stirred at room temperature for 1 h. To the stirred solution was added NaBH(OAc)₃ (0.29 g, 1.35 mmol) at room temperature under nitrogen atmosphere. The resulting solution was stirred at room temperature for 2 h. The reaction was quenched with water (20 mL) at room temperature and extracted with EA (5×30 mL). The combined organic layers were washed with brine (2×25 mL) and dried over anhydrous Na₂SO₄. After filtration, the filtrate was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 mL/min; Gradient: 10% B to 90% B in 9 min; Detector: UV 254/210 nm; Retention time: 8.10 min. The fractions containing desired product were collected and concentrated under reduced pressure to afford Compound 13 (N-([1-[(4,5-dichloro-2-hydroxyphenyl)methyl]-4-(hydroxymethyl)piperidin-4-yl]methyl)-2,2,2-trifluoroacetamide) as an off-white solid (82 mg, 43%): LCMS (ESI) calculated for C₁₆H₁₉Cl₂F₃N₂O₃ [M+H]⁺: 415, 417 (3:2), found 415, 417 (3:2); ¹H NMR (400 MHz, DMSO-d₆) δ 9.19 (s, 1H), 7.39 (s, 1H), 6.98 (s, 1H), 4.70 (br, 1H), 3.72 (s, 2H), 3.30 (s, 2H), 3.25 (d, J=6.0 Hz, 2H), 2.28-2.50 (m, 4H), 1.61-1.55 (m, 2H), 1.47-1.30 (m, 2H).

### Example 14. Compound 14 (2-[[4-amino-4-(hydroxymethyl)piperidin-1-yl]methyl]-4,5-dichlorophenol)

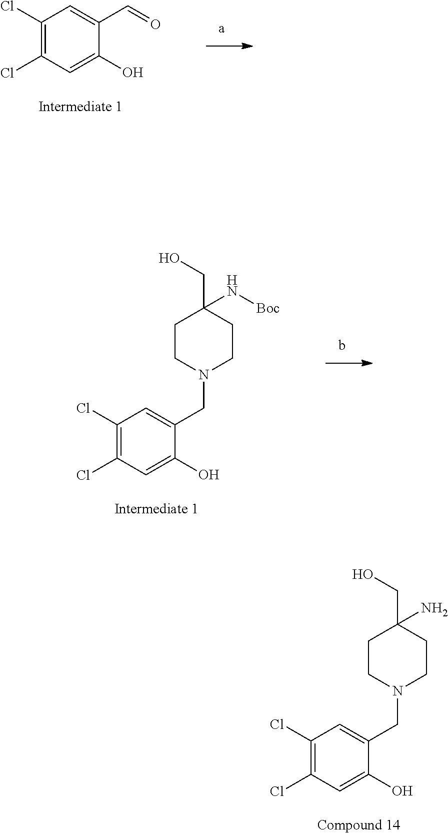 [SMILES: O=Cc1cc(Cl)c(Cl)cc1O ; CC(C)(C)OC(=O)NC1(CO)CCN(Cc2cc(Cl)c(Cl)cc2O)CC1 ; NC1(CO)CCN(Cc2cc(Cl)c(Cl)cc2O)CC1]

Step a:

To a stirred solution of Intermediate 1 (0.23 g, 1.20 mmol), HOAc (60 mg, 1.00 mmol) and tert-butyl-N-[4-(hydroxymethyl)piperidin-4-yl]carbamate (0.29 g, 1.00 mmol) in MeOH (5 mL) was added NaBH(OAc)₃ (0.64 g, 3.00 mmol) at room temperature under nitrogen atmosphere. The resulting mixture was stirred for 30 min at room temperature under nitrogen atmosphere, and then quenched with water (5 mL). The mixture was concentrated under reduced pressure. The residue was diluted with DCM (50 mL) and washed with water (3×20 mL). The organic phase was dried over anhydrous Na₂SO₄ and filtered. The filtrate was concentrated under reduced pressure. The residue was purified by silica gel column chromatography, eluted with EA to afford tert-butyl-N-[1-[(4,5-dichloro-2-hydroxyphenyl)methyl]-4-(hydroxymethyl)piperidin-4-yl]carbamate as a yellow oil (0.10 g, 25%): LCMS (ESI) calculated for C₁₈H₂₆Cl₂N₂O₄ [M+H]⁺: 405, 407 (3:2), found 405, 407 (3:2); ¹H NMR (400 MHz, CD₃OD) δ 7.23 (d, J=2.2 Hz, 1H), 6.89 (d, J=1.9 Hz, 1H), 3.74 (s, 2H), 3.61 (s, 2H), 2.80 (d, J=11.8 Hz, 2H), 2.45 (t, J=11.8 Hz, 2H), 2.12 (d, J=13.9 Hz, 2H), 1.73-1.62 (m, 2H), 1.45 (s, 9H).

Step b:

To a stirred solution of tert-butyl-N-[1-[(4,5-dichloro-2-hydroxyphenyl)methyl]-4-(hydroxymethyl) piperidin-4-yl]carbamate (0.10 g, 0.25 mmol) in DCM (2 mL) was added TFA (2 mL) at room temperature. The resulting mixture was stirred for 1 h at room atmosphere and concentrated under reduced pressure. The residue was purified by Prep-IPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 mL/min; Gradient: 20% B to 70% B in 9 min; Detector: UV 254/210 nm; Retention time: 7.41 min. The fractions containing desired product were collected and concentrated under reduced pressure to afford Compound 14 (2-[[4-amino-4-(hydroxymethyl)piperidin-1-yl]methyl]-4,5-dichlorophenol) as an off-white solid (29.3 mg, 39%): LCMS (ESI) calculated for C₁₃H₁₈Cl₂N₂O₂ [M+H]⁺: 305, 307 (3:2), found 305, 307 (3:2); ¹H NMR (400 MHz, CD₃OD) δ 7.18 (s, 1H), 6.85 (s, 1H), 3.72 (s, 2H), 3.38 (s, 2H), 2.68-2.55 (m, 4H), 1.71-1.65 (m, 2H), 1.57-1.47 (m, 2H).

### Example 15. Compound 15 (4,5-dichloro-2-[2-hydroxy-1-[4-(hydroxymethyl)piperidin-1-yl]ethyl]phenol)

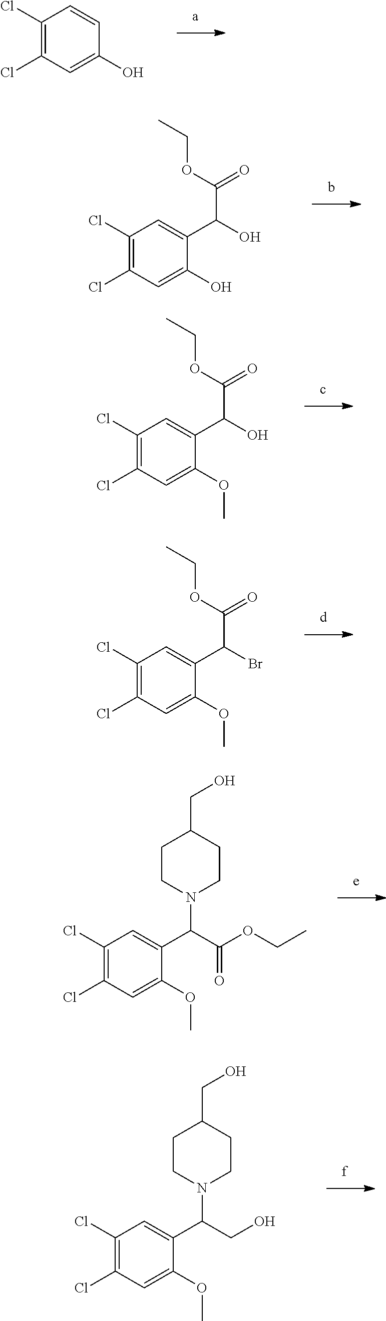

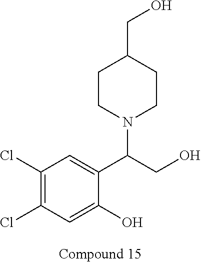 [SMILES: OCC1CCN(C(CO)c2cc(Cl)c(Cl)cc2O)CC1]

Step a:

To a stirred solution of 3,4-dichlorophenol (1.00 g, 6.13 mmol) in DCM (10 mL) was added TiCl₄ (1.20 g, 6.33 mmol) dropwise at −30° C. under argon atmosphere. After stirring at −30° C. for 30 min, a solution of ethyl 2-oxoacetate (1.50 g, 7.35 mmol, 50% in toluene) in DCM (5 mL) was added dropwise into the mixture. After addition, the resulting mixture was allowed to warm to room temperature and stirred for additional 16 h under argon atmosphere. The resulting solution was quenched with saturated aq. NH₄Cl (2 mL) at room temperature and diluted with co-solvent of EA (50 mL) and water (50 mL). The isolated aqueous solution was extracted with EA (3×50 mL). The combined organic layers were washed with brine (3×50 mL), dried over anhydrous Na₂SO₄ and filtered. The filtrate was concentrated under reduced pressure. The residue was purified by silica gel column chromatography, eluted with DCM/EA (6/1) to afford ethyl 2-(4,5-dichloro-2-hydroxyphenyl)-2-hydroxyacetate as a light yellow semi-solid (0.60 g, 31%): LCMS (ESI) calculated for C₁₀H₁₀Cl₂O₄ [M−1]⁺ 263, 265 (3:2), found 263, 265 (3:2); ¹H NMR (400 MHz, DMSO-d₆) δ 10.40 (s, 1H), 7.43 (s, 1H), 6.99 (s, 1H), 6.13 (d, J=8.0 Hz, 1H), 5.22 (s, 1H), 4.16-4.00 (m, 2H), 1.22-1.09 (m, 3H).

Step b:

To a stirred solution of ethyl 2-(4,5-dichloro-2-hydroxyphenyl)-2-hydroxyacetate (0.20 g, 0.75 mmol) in DMF (2 mL) was added K₂CO₃ (0.21 g, 1.51 mmol) and Mel (0.32 g, 2.26 mmol) at room temperature. The reaction mixture was allowed to warm to 40° C. and stirred for 1 h. The resulting mixture was diluted with EA (20 mL) and water (20 mL). The isolated aqueous layer was extracted with EA (3×20 mL). The combined organic layers were washed with brine (5×20 mL) and dried over anhydrous Na₂SO₄. After filtration, the filtrate was concentrated under reduced pressure. The residue was purified by silica gel column chromatography, eluted with PE/EA (5/1) to afford ethyl 2-(4,5-dichloro-2-methoxyphenyl)-2-hydroxyacetate as a colorless oil (0.15 g, 64%): ¹H NMR (300 MHz, DMSO-d₆) δ 7.51 (s, 1H), 7.29 (s, 1H), 6.22 (d, J=6.2 Hz, 1H), 5.21 (d, J=5.9 Hz, 1H), 4.08 (q, J=7.1 Hz, 2H), 3.80 (s, 3H), 1.13 (t, J=7.1 Hz, 3H).

Step c:

To a stirred solution of ethyl 2-(4,5-dichloro-2-methoxyphenyl)-2-hydroxyacetate (0.16 g, 0.57 mmol) in DCM (2 mL) was added PBr₃ (0.62 g, 2.29 mmol) dropwise at room temperature. The reaction solution was stirred at room temperature for 3 h. The resulting solution was quenched with water (20 mL) at room temperature and extracted with EA (3×20 mL). The combined organic layers were washed with brine (5×20 mL) and dried over anhydrous Na₂SO₄. After the filtration, the filtrate was concentrated under reduced pressure. The residue was purified by silica gel column chromatography, eluted with PE/EA (9/1) to afford ethyl 2-bromo-2-(4,5-dichloro-2-methoxyphenyl)acetate as a light yellow oil (0.15 g, 65%): ¹H NMR (300 MHz, CDCl₃) δ 7.73 (s, 1H), 7.11 (s, 1H), 5.70 (s, 1H), 4.25 (q, J=7.5 Hz, 2H), 3.85 (s, 3H), 1.27 (t, J=7.1 Hz, 3H).

Step d:

To a stirred solution of ethyl 2-bromo-2-(4,5-dichloro-2-methoxyphenyl)acetate (0.15 g, 0.44 mmol) in DMF (2 mL) was added piperidin-4-ylmethanol (76 mg, 0.66 mmol) and K₂CO₃ (0.12 g, 0.88 mmol) at room temperature. The reaction mixture was allowed to warm to 40° C. and stirred for 2 h. The resulting mixture was diluted with co-solvent of EA (20 mL) and water (20 mL). The isolated aqueous layer was extracted with EA (3×20 mL). The combined organic layers were washed with brine (5×20 mL) and dried over anhydrous Na₂SO₄. After the filtration, the filtrate was concentrated under reduced pressure to afford ethyl 2-(4,5-dichloro-2-methoxyphenyl)-2-[4-(hydroxymethyl)piperidin-1-yl]acetate as a light yellow oil (0.15 g, crude): LCMS (ESI) calculated for C₁₇H₂₃Cl₂NO₄ [M+H]⁺: 376, 378 (3:2), found 376, 378 (3:2); ¹H NMR (300 MHz, CDCl₃) 7.59 (s, 1H), 6.95 (s, 1H), 4.52 (s, 1H), 4.18 (q, J=9.0, 2H), 3.81 (s, 3H), 3.52 (d, J=7.1 Hz, 2H), 3.10-2.98 (m, 2H), 2.37-2.02 (m, 2H), 1.81-1.61 (m, 2H) 1.60-1.40 (m, 3H), 1.24 (q, J=7.2 Hz, 3H).

Step e:

To a stirred solution of ethyl 2-(4,5-dichloro-2-methoxyphenyl)-2-[4-(hydroxymethyl) piperidin-1-yl]acetate (0.14 g, 0.37 mmol) in THE (2 mL) was added DIBAL-H (2.2 mL, 2.21 mmol, 1 M in toluene) at 0° C. under argon atmosphere. The reaction solution was stirred at 0° C. for 1 h under argon atmosphere. The resulting solution was quenched with water (20 mL) at 0° C. and extracted with EA (3×20 mL). The combined organic layers were washed with brine (3×20 mL) and dried over anhydrous Na₂SO₄. After the filtration, the filtrate was concentrated under reduced pressure to afford 2-(4,5-dichloro-2-methoxyphenyl)-2-[4-(hydroxymethyl)piperidin-1-yl]ethan-1-ol as a light yellow oil (0.10 g, crude), which was used in next step without further purification: LCMS (ESI) calculated for C₁₅H₂₁Cl₂NO₃ [M+H]⁺: 334, 335 (3:2), found 334, 335 (3:2).

Step f:

To a stirred solution of 2-(4,5-dichloro-2-methoxyphenyl)-2-[4-(hydroxymethyl) piperidin-1-yl] ethan-1-ol (0.10 g, 0.30 mmol) in DCM (2 mL) was added BBr₃ (0.34 g, 1.35 mmol) at room temperature. The reaction solution was stirred at room temperature for 5 h. The resulting mixture was quenched with water (1 mL) at 0° C. and concentrated under reduced pressure. The residue was purified by Prep-HPLC with following conditions: Column: XBridge C₁₈ OBD Prep Column 100 Å, 10 μm, 19 mm×250 mm; Mobile Phase A: water with 20 mmoL/L NH₄HCO₃, Mobile Phase B: ACN; Flow rate: 20 mL/min; Gradient: 40% B to 90% B in 9 min; Detector: UV 254/210 nm; Retention time: 6.55 min. The fractions containing desired product were collected and concentrated under reduced pressure to afford Compound 15 (4,5-dichloro-2-[2-hydroxy-1-[4-(hydroxymethyl)piperidin-1-yl]ethyl]phenol) as an off-white solid (20 mg, 20%): LCMS (ESI) calculated for C₁₄H₁₉Cl₂NO₃ [M+H]⁺: 320, 322 (3:2), found 320, 322 (3:2); ¹H NMR (400 MHz, CD₃OD) δ 7.24 (s, 1H), 6.86 (s, 1H), 4.00-3.87 (m, 2H), 3.63 (t, J=4.8 Hz, 1H), 3.43 (d, J=6.3 Hz, 2H), 3.33-3.28 (m, 1H), 3.07-2.98 (m, 1H), 2.40-2.29 (m, 2H), 1.92-1.76 (m, 2H), 1.57 (s, 1H), 1.40-1.23 (m, 2H).

### Example 16. Compound 16 (2-[[4-(hydroxymethyl)piperidin-1-yl]methyl]-4,5-dimethylphenol)

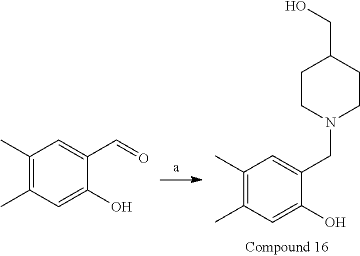 [SMILES: Cc1cc(O)c(C=O)cc1C ; Cc1cc(O)c(CN2CCC(CO)CC2)cc1C]

Step a:

To a stirred solution of 2-hydroxy-4,5-dimethylbenzaldehyde (0.10 g, 0.67 mmol) and piperidin-4-ylmethanol (77 mg, 0.67 mmol) in MeOH (3 mL) were added HOAc (40 mg, 0.67 mmol) and NaBH(OAc)₃ (0.42 g, 2.00 mmol) at room temperature under nitrogen atmosphere. After stirring at room temperature under nitrogen atmosphere for 2 h, the resulting mixture was quenched with water (3 mL) and concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 mL/min; Gradient: 30% B to 55% B in 9 min; Detector: UV 254/210 nm; Retention time: 8.15 min. The fractions containing desired product were collected and concentrated under reduced pressure to afford Compound 16 (2-[[4-(hydroxymethyl)piperidin-1-yl]methyl]-4,5-dimethylphenol) as an off-white solid (25 mg, 15%): LCMS (ESI) calculated for C₁₅H₂₃NO₂ [M+H]⁺: 250, found 250; ¹H NMR (400 MHz, DMSO-d₆) δ 10.61 (br, 1H), 6.78 (s, 1H), 6.51 (s, 1H), 4.44 (br, 1H), 3.55 (s, 2H), 3.26 (d, J=6.2 Hz, 2H), 2.88 (d, J=11.7, 2H), 2.10 (d, J=11.5 Hz, 6H), 1.99 (td, J=11.6, 2.5 Hz, 2H), 1.73-1.63 (m, 2H), 1.45-1.35 (m, 1H), 1.21-1.05 (m, 2H).

### Example 17. Compound 17 (5-chloro-2-[[4-(hydroxymethyl)piperidin-1-yl]methyl]-4-methylphenol)

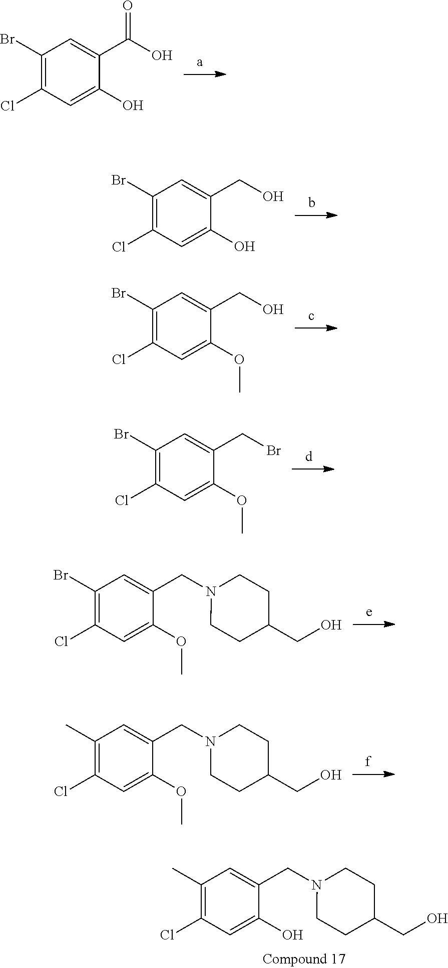

Step a:

To a stirred solution of 5-bromo-4-chloro-2-hydroxybenzoic acid (0.50 g, 1.99 mmol) in THE (10 mL) was added BH₃ (6 mL, 6.00 mmol, 1 M in THF) dropwise at 0° C. under nitrogen atmosphere. Then the reaction solution was allowed to warm to room temperature and stirred for 1 h under nitrogen atmosphere. The resulting solution was quenched with water (30 mL) at 0° C. and extracted with EA (3×30 mL). The combined organic layers were washed with brine (2×20 mL), dried over anhydrous Na₂SO₄ and filtered. The filtrate was concentrated under reduced pressure to afford 4-bromo-5-chloro-2-(hydroxymethyl)phenol as an off-white solid (0.33 g, 69%): LCMS (ESI) calculated for C₇H₆BrClO₂ [M−H]⁺: 235, 237, 239 (2:3:1), found 235, 237, 239 (2:3:1); ¹H NMR (300 MHz, CDCl₃) δ 7.34 (s, 1H), 6.89 (s, 1H), 4.69 (s, 2H).

Step b:

To a stirred mixture of 4-bromo-5-chloro-2-(hydroxymethyl)phenol (0.33 g, 1.41 mmol) and K₂CO₃ (0.39 g, 2.81 mmol) in DMF (3.5 mL) was added Mel (0.60 g, 4.22 mmol) dropwise at 25° C. The reaction mixture was stirred at 25° C. for 2 h. The resulting mixture was diluted with water (20 mL) and extracted with EA (3×30 mL). The combined organic layers were washed with brine (2×20 mL), dried over anhydrous Na₂SO₄ and filtered. The filtrate was concentrated under reduced pressure. The residue was purified by silica gel column chromatography, eluted with PE/EA (15/1) to afford (5-bromo-4-chloro-2-methoxyphenyl)methanol as an off-white solid (0.20 g, 56%): ¹H NMR (300 MHz, CDCl₃) δ 7.56 (s, 1H), 6.99 (s, 1H), 4.66 (s, 2H), 3.90 (s, 3H).

Step c:

To a stirred solution of (5-bromo-4-chloro-2-methoxyphenyl)methanol (0.20 g, 0.79 mmol) in DCM (3.5 mL) was added PBr₃ (0.43 g, 1.58 mmol) at 25° C. under nitrogen atmosphere. After stirring for 1 h at 25° C., the resulting solution was quenched with water (30 mL) and extracted with EA (3×30 mL). The combined organic layers were washed with brine (2×20 mL), dried over anhydrous Na₂SO₄ and filtered. The filtrate was concentrated under reduced pressure to afford 1-bromo-5-(bromomethyl)-2-chloro-4-methoxybenzene as an off-white solid (0.20 g, 80%), which was directly used in next step without further purification: ¹H NMR (300 MHz, CDCl₃) δ 7.55 (s, 1H), 6.97 (s, 1H), 5.06 (s, 2H), 3.90 (s, 3H).

Step d:

To a mixture of 1-bromo-5-(bromomethyl)-2-chloro-4-methoxybenzene (0.20 g, 0.76 mmol) and K₂CO₃ (0.21 g, 1.51 mmol) in DMF (2.5 mL) was added piperidin-4-ylmethanol (0.13 g, 1.13 mmol) at room temperature. The reaction mixture was allowed to warm to 40° C. and stirred at for 1.5 h. After cooling to room temperature, the resulting mixture was diluted with water (20 mL) and extracted with EA (3×30 mL). The combined organic layers were washed with brine (2×20 mL) and dried over anhydrous Na₂SO₄ and filtered. The filtrate was concentrated under reduced pressure. The residue was purified by silica gel column chromatography, eluted with DCM/MeOH (15/1) to afford [1-[(5-bromo-4-chloro-2-methoxyphenyl)methyl]piperidin-4-yl]methanol as an off-white solid (0.13 g, 49%): LCMS (ESI) calculated for C₁₄H₁₉BrClNO₂ [M+H]⁺: 348, 350, 352 (2:3:1), found 348, 350, 352 (2:3:1).

Step e:

To a mixture of [1-[(5-bromo-4-chloro-2-methoxyphenyl)methyl]piperidin-4-yl]methanol (0.13 g, 0.37 mmol), methylboronic acid (66 mg, 1.11 mmol) and K₂CO₃ (0.23 g, 1.67 mmol) in 1,4-dioxane (4 mL) and H₂O (1 mL) was added Pd(dppf)Cl₂ (54 mg, 0.07 mmol) at room temperature. The reaction mixture was degassed with nitrogen for three times. Then reaction mixture was allowed to warm to 80° C. and stirred for 2.5 h under nitrogen atmosphere. After cooling to room temperature, the resulting mixture was quenched with water (20 mL) and extracted with EA (3×30 mL). The combined organic layers were washed with brine (2×20 mL), dried over anhydrous Na₂SO₄ and filtered. The filtrate was concentrated under reduced pressure. The residue was purified by silica gel column chromatography, eluted with DCM/MeOH (10/1) to afford [1-[(4-chloro-2-methoxy-5-methylphenyl)methyl]piperidin-4-yl]methanol as a brown solid (72 mg, 68%): LCMS (ESI) calculated for C₁₅H₂₂ClNO₂ [M+H]⁺: 284, 286 (3:1), found 284, 286 (3:1); ¹H NMR (300 MHz, CDCl₃) δ 7.55 (s, 1H), 6.91 (s, 1H), 4.07 (s, 2H), 3.79 (s, 3H), 3.55-3.49 (m, 3H), 3.31 (s, 2H), 2.49 (s, 1H), 2.30 (s, 3H), 1.85-1.23 (m, 5H).

Step f:

To a stirred solution of [1-[(4-chloro-2-methoxy-5-methylphenyl)methyl]piperidin-4-yl]methanol (72 mg, 0.25 mmol) in DCM (2.5 mL) was added BBr₃ (0.25 g, 1.01 mmol) at room temperature. After stirring at room temperature for 2.5 h, the resulting mixture was quenched with water (8 mL) at room temperature and adjusted pH value to 7 with saturated aq. NaHCO₃. The aqueous layer was extracted with EA (3×30 mL). The combined organic layers were washed with brine (2×20 mL), dried over anhydrous Na₂SO₄ and filtered. The filtrate was concentrated under reduced pressure. The residue was purified by Prep-HPLC with the following conditions: Column: X Bridge C₁₈ OBD Prep Column 100 Å, 10 μm, 19 mm×250 mm; Mobile Phase A: water with 20 mmoL/L NH₄HCO₃, Mobile Phase B: ACN; Flow rate: 20 mL/min; Gradient: 10% B to 90% B in 9 min; Detector: UV 254/210 nm; Retention time: 8.17 min. The fractions containing desired product were collected and concentrated under reduced pressure to afford Compound 17 (5-chloro-2-[[4-(hydroxymethyl)piperidin-1-yl]methyl]-4-methylphenol) as an off-white solid (16 mg, 23%): LCMS (ESI) calculated for C₁₄H₂₀ClNO₂ [M+H]⁺: 270, 272 (3:1), found 270, 272 (3:1); ¹H NMR (300 MHz, CD₃OD) δ 6.93 (s, 1H), 6.73 (s, 1H), 3.69 (s, 2H), 3.42 (d, J=6.3 Hz, 2H), 3.04 (d, J=11.4 Hz, 2H), 2.23 (s, 3H), 2.21-2.15 (m, 2H), 1.82 (d, J=13.2 Hz, 2H), 1.60-1.49 (m, 1H), 1.35-1.22 (m, 2H).

### Example 18. Compound 18 (4,5-dichloro-2-[1-[4-(hydroxymethyl)piperidin-1-yl]propyl]phenol)

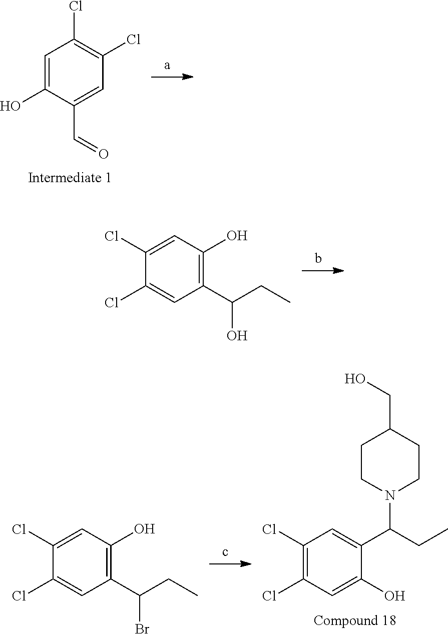 [SMILES: O=Cc1cc(Cl)c(Cl)cc1O ; CCC(O)c1cc(Cl)c(Cl)cc1O ; CCC(Br)c1cc(Cl)c(Cl)cc1O ; CCC(c1cc(Cl)c(Cl)cc1O)N1CCC(CO)CC1]

Step a:

To a stirred solution of Intermediate 1 (0.15 g, 0.79 mmol) in THE (3 mL) was added bromo(ethyl)magnesium (0.6 mL, 1.74 mmol, 3 M in ether) at room temperature under nitrogen atmosphere. After stirring for 2 h at room temperature under nitrogen atmosphere, the resulting solution was quenched with water (30 mL) at 0° C. and extracted with EA (3×35 mL). The combined organic layers were washed with brine (2×20 mL), dried over anhydrous Na₂SO₄ and filtered. The filtrate was concentrated under reduced pressure to afford 2-(1-bromopropyl)-4,5-dichlorophenol as an off-white solid (72 mg, crude), which was used in next step directly without further purification: LCMS (ESI) calculated for C₉H₁₀Cl₂O₂ [M−H]⁺: 219, 221 (3:2), found 219, 221 (3:2).

Step b:

To a stirred solution of 4,5-dichloro-2-(1-hydroxypropyl)phenol (0.20 g, 0.90 mmol) in DCM (3 mL) was added PBr₃ (0.49 g, 1.81 mmol) at room temperature under nitrogen atmosphere. After stirring for 2 h at room temperature under nitrogen atmosphere, the resulting solution was quenched with water (30 mL) and extracted with EA (3×45 mL). The combined organic layers were washed with brine (3×20 mL) and dried over anhydrous Na₂SO₄. After the filtration, the filtrate was concentrated under reduced pressure. The residue was purified by Prep-HPLC with the following conditions: Column: XBridge C₁₈ OBD Prep Column 100 Å, 10 m, 19 mm×250 mm; Mobile Phase A: water with 20 mmol/L NH₄HCO₃, Mobile Phase B: ACN; Flow rate: 20 mL/min; Gradient: 30% B to 80% B in 9 min; Detector: UV 254/210 nm; Retention time: 7.50 min. The fractions containing desired product were collected and concentrated under reduced pressure to afford 2-(1-bromopropyl)-4,5-dichlorophenol as an off-white solid (70 mg, 32% overall two steps): LCMS (ESI) calculated for C₉H₉BrCl₂O [M−H]⁺: 281, 283, 285 (2:3:1), found 281, 283, 285 (2:3:1).

Step c:

To a stirred mixture of 2-(1-bromopropyl)-4,5-dichlorophenol (70 mg, 0.25 mmol) and K₂CO₃ (69 mg, 0.49 mmol) in DMF (3 mL) was added piperidin-4-ylmethanol (28 mg, 0.25 mmol) at room temperature. After stirring for 2 h at room temperature, the resulting mixture was diluted with water (20 mL) and extracted with EA (5×20 mL). The combined organic layers were dried over anhydrous Na₂SO₄ and filtered. The filtrate was concentrated under reduced pressure. The residue was purified by Prep-HPLC with the following conditions: Column: XBridge C₁₈ OBD Prep Column, 100 Å, 5 μm, 19 mm×250 mm; Mobile Phase A: water with 20 mmol/L NH₄HCO₃, Mobile Phase B: ACN; Flow rate: 20 mL/min; Gradient: 30% B to 80% B in 9 min; Detector: UV 254/210 nm; Retention time: 7.54 min. The fractions containing desired product were collected and concentrated under reduced pressure to afford Compound 18 (4,5-dichloro-2-[1-[4-(hydroxymethyl)piperidin-1-yl]propyl]phenol) as an off-white solid (18 mg, 21%): LCMS (ESI) calculated for C₁₅H₂₁Cl₂NO₂ [M+H]⁺: 318, 320 (3:2), found 318, 320 (3:2); ¹H NMR (300 MHz, DMSO-d₆) δ 7.24 (s, 1H), 6.90 (s, 1H), 3.60-3.52 (m, 1H), 3.21 (d, J=6.1 Hz, 2H), 3.06 (d, J=11.5 Hz, 1H), 2.84 (d, J=11.5 Hz, 1H), 2.09-1.89 (m, 2H), 1.89-1.52 (m, 4H), 1.43-1.34 (m, 1H), 1.26-1.01 (m, 2H), 0.68 (t, J=7.3 Hz, 3H).

### Example 19. Compound 19 (4-chloro-2-[[4-(hydroxymethyl)piperidin-1-yl]methyl]-5-methylphenol)

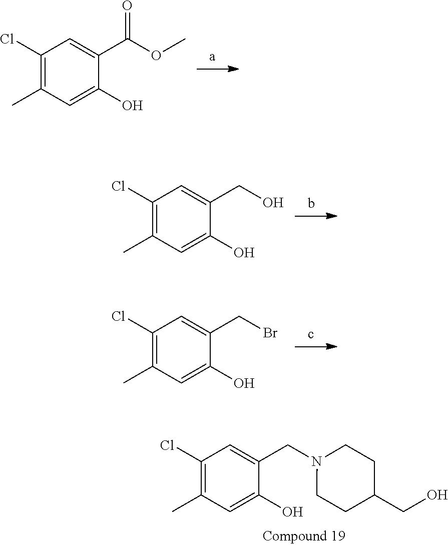

Step a:

To a stirred solution of methyl 5-chloro-2-hydroxy-4-methylbenzoate (0.50 g, 2.49 mmol) in THE (15 mL) was added DIBAL-H (12.5 mL, 12.46 mmol, 1 M in toluene) dropwise at 0° C. under nitrogen atmosphere. The resulting mixture was stirred at 0° C. under nitrogen atmosphere for 2 h. The reaction mixture was quenched with water (50 mL) at 0° C. and extracted with EA (3×50 mL). The combined organic layers were washed with brine (3×50 mL), dried over anhydrous Na₂SO₄ and filtered. The filtrate was concentrated under reduced pressure. The residue was purified by silica gel column chromatography, eluted with PE/EA (5/1) to afford 4-chloro-2-(hydroxymethyl)-5-methylphenol as an off-white solid (0.35 g, 67%): LCMS (ESI) calculated for C₈H₉ClO₂ [M−1]⁻: 171, 173 (3:1), found 171, 173 (3:1); ¹H NMR (300 MHz, CDCl₃) δ 7.00 (s, 1H), 6.77 (s, 1H), 4.81 (s, 2H), 2.31 (s, 3H).

Step b:

To a stirred solution of 4-chloro-2-(hydroxymethyl)-5-methylphenol (0.35 g, 2.03 mmol) in DCM (10 mL) was added PBr₃ (1.10 g, 4.06 mmol) dropwise at 0° C. under nitrogen atmosphere. After stirring for 2 h at 0° C. under nitrogen atmosphere, the resulting solution was quenched with water (30 mL) at 0° C. and extracted with EA (3×70 mL). The combined organic layers were washed with brine (3×30 mL), dried over anhydrous Na₂SO₄ and filtered. The filtrate was concentrated under reduced pressure to afford 2-(bromomethyl)-4-chloro-5-methylphenol as a yellow oil (0.35 g, crude), which was directly used in the next step without further purification: LCMS (ESI) calculated for C₈H₈BrClO [M−H]⁺: 233, 235, 237 (2:3:1), found 233, 235, 237 (2:3:1).

Step c:

To a stirred mixture of 2-(bromomethyl)-4-chloro-5-methylphenol (0.35 g, 1.49 mmol) and K₂CO₃ (0.41 g, 2.97 mmol) in ACN (15 mL) was added piperidin-4-ylmethanol (0.26 g, 2.23 mmol) at room temperature. The reaction mixture was allowed to warm to 40° C. and stirred for 16 h. The resulting mixture was diluted with water (30 mL) and extracted with EA (3×40 mL). The combined organic layers were washed with brine (3×30 mL), dried over anhydrous Na₂SO₄ and filtered. The filtrate was concentrated under reduced pressure. The residue was purified by Prep-HPLC with the following conditions: Column: X Bridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 mL/min; Gradient: 20% B to 60% B in 9 min; Detector: UV 254/210 nm; Retention time: 8.50 min. The fractions containing desired product were collected and concentrated under reduced pressure to afford Compound 19 (4-chloro-2-[[4-(hydroxymethyl)piperidin-1-yl]methyl]-5-methylphenol) as an off-white solid (25 mg, 6% overall two steps): LCMS (ESI) calculated for C₁₄H₂₀ClNO₂ [M+H]⁺: 270, 272 (3:1), found 270, 272 (3:1); ¹H NMR (300 MHz, DMSO-d₆) δ 7.06 (s, 1H), 6.67 (s, 1H), 3.56 (s, 2H), 3.23 (d, J=6.2 Hz, 2H), 2.84 (d, J=11.2 Hz, 2H), 2.19 (s, 3H), 2.00 (m, J=11.2, 2.4 Hz, 2H), 1.65 (d, J=13.0 Hz, 2H), 1.35 (d, J=11.2 Hz, 1H), 1.22-0.98 (m, 2H).

### Example 20. Compound 21 (N-([1-[(4,5-dichloro-2-hydroxyphenyl)methyl]-4-(hydroxymethyl)piperidin-4-yl]methyl)acetamide)

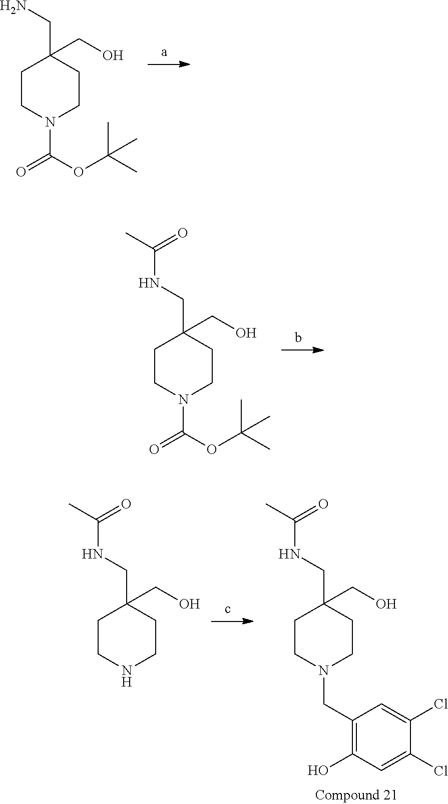 [SMILES: CC(C)(C)OC(=O)N1CCC(CN)(CO)CC1 ; CC(=O)NCC1(CO)CCN(C(=O)OC(C)(C)C)CC1 ; CC(=O)NCC1(CO)CCNCC1 ; CC(=O)NCC1(CO)CCN(Cc2cc(Cl)c(Cl)cc2O)CC1]

Step a:

To a stirred solution of tert-butyl 4-(aminomethyl)-4-(hydroxymethyl)piperidine-1-carboxylate (0.20 g, 0.82 mmol) and Et₃N (0.25 g, 2.46 mmol) in DCM (1 mL) was added acetic anhydride (84 mg, 0.82 mmol) at room temperature. The resulting solution was stirred at room temperature for 1 h. The resulting solution was concentrated under reduced pressure to afford tert-butyl 4-(acetamidomethyl)-4-(hydroxymethyl)piperidine-1-carboxylate (0.30 g, crude), which was used in next step without further purification: LCMS (ESI) calculated for C₁₄H₂₆N₂O₄ [M+H]⁺: 287, found 287.

Step b:

To a stirred solution of tert-butyl 4-(acetamidomethyl)-4-(hydroxymethyl)piperidine-1-carboxylate (0.30 g, 1.05 mmol) in DCM (1 mL) was added TFA (1 mL) at room temperature. The reaction solution was stirred at room temperature for 30 min. The resulting solution was concentrated under reduced pressure. The residue was dissolved in water (5 mL) and neutralized to pH 8 with saturated aq. NaHCO₃. The aqueous layer was extracted with EA (10×20 mL). The combined organic layers were dried over anhydrous Na₂SO₄ and filtered. The filtrate was concentrated under reduced pressure to afford N-((4-(hydroxymethyl)piperidin-4-yl)methyl)acetamide (0.12 g, crude), which was used in next step without further purification: LCMS (ESI) calculated for C₉H₁₈N₂O₂ [M+H]⁺: 187, found 187.

Step c:

To a stirred solution of N-[[4-(hydroxymethyl)piperidin-4-yl]methyl]acetamide (0.12 g, 0.58 mmol) and Intermediate 1 (0.11 g, 0.58 mmol) in MeOH (1 mL) was added HOAc (35 mg, 0.6 mmol) and NaBH(OAc)₃ at room temperature under nitrogen atmosphere. The reaction solution was stirred at room temperature for 2 h under nitrogen atmosphere. The resulting solution was quenched with water (5 mL) at room temperature and concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 mL/min; Gradient: 30% B to 80% B in 9 min; Detector: UV 254/210 nm; Retention time: 8.14 min. The fractions containing desired product were collected and concentrated under reduced pressure to afford Compound 21 (N-([1-[(4,5-dichloro-2-hydroxyphenyl)methyl]-4-(hydroxymethyl)piperidin-4-yl]methyl)acetamide) as an off-white solid (97 mg, 46%): LCMS (ESI) calculated for C₁₆H₂₂Cl₂N₂O₃ [M+H]⁺: 361, 363 (3:2), found 361, 363 (3:2); ¹H NMR (400 MHz, DMSO-d₆) δ 7.89-7.76 (m, 1H), 7.34 (s, 1H), 6.95 (s, 1H), 4.94-4.29 (m, 1H). 3.64 (s, 2H), 3.07 (d, J=6.3 Hz, 2H), 2.49-2.40 (m, 4H), 1.86 (s, 3H), 1.47-1.26 (m, 4H).

### Example 21. Compound 23 (4,5-dichloro-2-[1-[4-(hydroxymethyl)piperidin-1-yl]-3-methylbutyl]phenol)

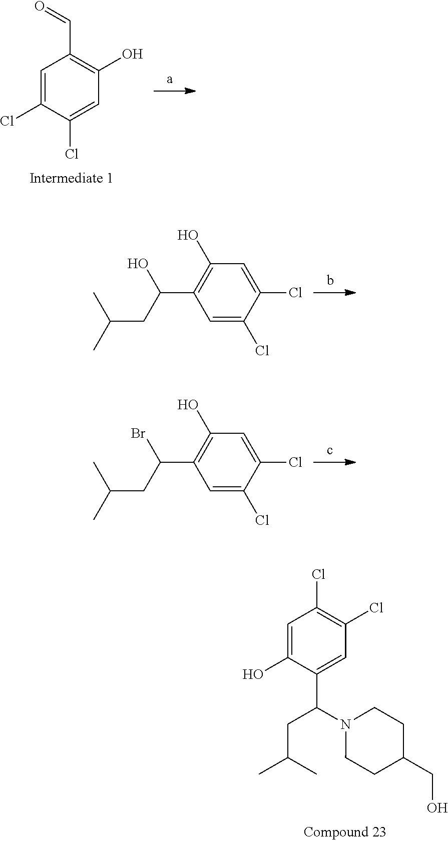 [SMILES: O=Cc1cc(Cl)c(Cl)cc1O ; CC(C)CC(O)c1cc(Cl)c(Cl)cc1O ; CC(C)CC(Br)c1cc(Cl)c(Cl)cc1O ; CC(C)CC(c1cc(Cl)c(Cl)cc1O)N1CCC(CO)CC1]

Step a:

To a stirred solution of Intermediate 1 (0.10 g, 0.52 mmol) in THE (2 mL) was added bromo(2-methylpropyl)magnesium (0.6 mL, 1.14 mmol, 2 M in ether) at room temperature under nitrogen atmosphere. After stirring for 1 h, the resulting solution was quenched with water (20 mL) and extracted with EA (2×30 mL). The combined organic layers were washed with brine (2×20 mL) and dried over anhydrous Na₂SO₄. After the filtration, the filtrate was concentrated under reduced pressure to afford 4,5-dichloro-2-(1-hydroxy-3-methylbutyl)phenol as a yellow oil (0.14 g, crude), which was used in next step directly without further purification: LCMS (ESI) calculated for C₁₁H₁₄Cl₂O₂ [M−H]⁺: 247, 249 (3:2), found 247, 249 (3:2).

Step b:

To a stirred solution of 4,5-dichloro-2-(1-hydroxy-3-methylbutyl)phenol (0.14 g, crude) in DCM (2 mL) was added PBr₃ (0.30 g, 1.12 mmol) at room temperature at nitrogen atmosphere. The reaction solution was stirred for 2 h at room temperature under nitrogen atmosphere. The resulting solution was quenched with water (20 mL) and extracted with EA (3×30 mL). The combined organic layers were washed with brine (2×20 mL), dried over anhydrous Na₂SO₄. After filtration, the filtrate was concentrated under reduced pressure to afford 2-(1-bromo-3-methylbutyl)-4,5-dichlorophenol as a yellow oil (0.18 g, crude), which was used in next step without further purification: LCMS (ESI) calculated for C₁₁H₁₃BrCl₂₀ [M−H]⁺: 309, 311, 313 (2:3:1), found 309, 311, 313 (2:3:1).

Step c:

To a stirred solution of 2-(1-bromo-3-methylbutyl)-4,5-dichlorophenol (0.18 g, 0.58 mmol) in DMF (1 mL) were added piperidin-4-ylmethanol (0.13 g, 1.15 mmol) and K₂CO₃ (0.16 g, 1.15 mmol) at room temperature. After stirring for 2 h at room temperature, the resulting mixture was diluted with water (20 mL) at room temperature and extracted with EA (5×50 mL). The combined organic layers were washed with brine (2×20 mL) and dried over anhydrous Na₂SO₄. After filtration, the filtrate was concentrated under reduced pressure. The residue was purified by Prep-HPLC with the following conditions: Column: XBridge C₁₈ OBD Prep Column 100 Å, 10 μm, 19 mm×250 mm; Mobile Phase A: water with 20 mmol/L NH₄HCO₃, Mobile Phase B: ACN; Flow rate: 20 mL/min; Gradient: 20% B to 80% B in 9 min; Detector: UV 254/210 nm; Retention time: 8.14 min. The fractions containing desired product were collected and concentrated under reduced pressure to afford Compound 23 (4,5-dichloro-2-[1-[4-(hydroxymethyl)piperidin-1-yl]-3-methylbutyl]phenol) as an off-white solid (10 mg, 5% overall three steps): LCMS (ESI) calculated for C₁₇H₂₅Cl₂NO₂ [M+H]⁺: 346, 348 (3:2), found 346, 348 (3:2); ¹H NMR (400 MHz, DMSO-d₆) δ 7.25 (s, 1H), 6.96 (s, 1H), 4.39 (br, 1H), 3.83-3.76 (m, 1H), 3.22 (d, J=6.1 Hz, 2H), 3.00 (d, J=11.5 Hz, 1H), 2.83 (d, J=11.5 Hz, 1H), 1.98-1.89 (m, 2H), 1.78-1.60 (m, 3H), 1.57-1.48 (m, 1H), 1.43-1.23 (m, 2H), 1.19-0.99 (m, 2H), 0.92-0.82 (m, 6H).

### Example 22. Compound 28 (2-(1-(4,5-dichloro-2-hydroxybenzyl)piperidin-4-yl)acetic acid)

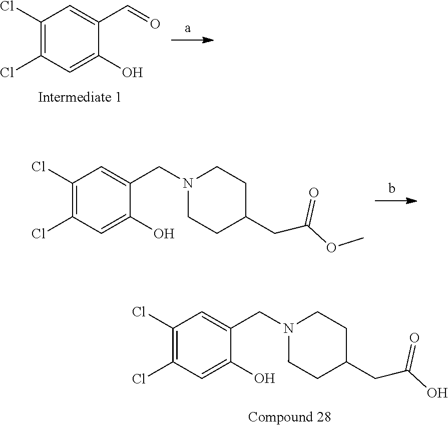 [SMILES: O=Cc1cc(Cl)c(Cl)cc1O ; COC(=O)CC1CCN(Cc2cc(Cl)c(Cl)cc2O)CC1 ; O=C(O)CC1CCN(Cc2cc(Cl)c(Cl)cc2O)CC1]

Step a:

To a stirred solution of methyl 2-(piperidin-4-yl)acetate (0.25 g, 1.29 mmol) and Intermediate 1 (0.20 g, 1.05 mmol) in MeOH (3 mL) were added HOAc (62 mg, 1.03 mmol) and NaBH(OAc)₃ (0.66 g, 3.12 mmol) at room temperature under nitrogen atmosphere. After stirring for 2 h at room temperature under nitrogen atmosphere, the resulting solution was quenched with water (3 mL) and concentrated under reduced pressure. The residue was purified by silica gel column chromatography, eluted with PE/EA (7/1) to afford methyl 2-(1-(4,5-dichloro-2-hydroxybenzyl)piperidin-4-yl)acetate as a light brown solid (0.19 g, 55%): LCMS (ESI) calculated for C₁₅H₁₉C₁₂NO₃ [M+H]⁺: 332, 334 (3:2), found 332, 334 (3:2); ¹H NMR (400 MHz, CDCl₃) δ 7.05 (d, J=0.9 Hz, 1H), 6.94 (s, 1H), 3.69 (s, 3H), 3.67 (s, 2H), 3.00 (d, J=11.7 Hz, 2H), 2.29 (d, J=6.9 Hz, 2H), 2.24-2.13 (m, 2H), 1.96-1.75 (m, 3H), 1.44-1.31 (m, 2H).

Step b:

To a stirred solution of methyl 2-[1-[(4,5-dichloro-2-hydroxyphenyl)methyl]piperidin-4-yl]acetate (0.19 g, 0.57 mmol) in MeOH (4 mL) and water (2 mL) was added NaOH (0.11 g, 2.75 mmol) at room temperature. The reaction solution was stirred at room temperature for 2 h. The resulting solution was adjusted pH to 7-8 by aq. HCl (1 N). The resulting mixture was concentrated under reduced pressure. The residue was purified by Prep-HPLC with the following conditions: Column: Sunfire Prep C₁₈ OBD Column, 10 μm, 19×250 mm; Mobile Phase A: water (plus 0.05% TFA), Mobile Phase B: ACN; Flow rate: 20 mL/min; Gradient: 16% B to 43% B in 9 min; Detector: UV 254/210 nm; Retention time: 7.52. The fractions containing desired product were collected and concentrated under reduced pressure to afford Compound 28 2-(1-(4,5-dichloro-2-hydroxybenzyl)piperidin-4-yl)acetic acid trifluoroacetic acid as a colorless viscous oil (24.7 mg, 14%): LCMS (ESI) calculated for C₁₄H₁₇Cl₂NO₃ [M+H]⁺: 318, 320 (3:2), found 318, 320 (3:2); ¹H NMR (400 MHz, CD₃OD) δ 7.58 (s, 1H), 7.12 (s, 1H), 4.27 (s, 2H), 3.58-3.49 (m, 2H), 3.30 (s, 1H), 3.14-3.04 (m, 2H), 2.31 (d, J=6.5 Hz, 2H), 2.14-2.00 (m, 2H), 1.53 (m, 2H).

### Example 23. Compound 29 (4,5-dichloro-2-(((2S,4S)-rel-4-(hydroxymethyl)-2-methylpiperidin-1-yl)methyl)phenol) and Compound 24 (4,5-dichloro-2-(((2R,4S)-rel-4-(hydroxymethyl)-2-methylpiperidin-1-yl)methyl)phenol)

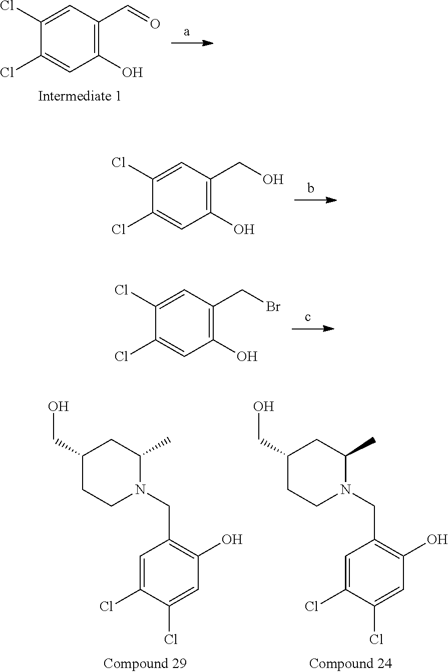 [SMILES: O=Cc1cc(Cl)c(Cl)cc1O ; OCc1cc(Cl)c(Cl)cc1O ; Oc1cc(Cl)c(Cl)cc1CBr ; C[C@H]1C[C@@H](CO)CCN1Cc1cc(Cl)c(Cl)cc1O ; C[C@@H]1C[C@@H](CO)CCN1Cc1cc(Cl)c(Cl)cc1O]

Step a:

To a stirred solution of Intermediate 1 (0.20 g, 1.05 mmol) in EtOH (10 mL) was added NaBH₄ (79 mg, 2.09 mmol) at 0° C. under nitrogen atmosphere. The reaction mixture was stirred at 0° C. for 30 min under nitrogen atmosphere. The resulting mixture was quenched with water (10 mL) and extracted with EA (3×30 mL). The combined organic layers were washed with brine (2×20 mL), dried over anhydrous Na₂SO₄ and filtered. The filtrate was concentrated under reduced pressure to afford 4,5-dichloro-2-(hydroxymethyl)phenol as an off-white solid (0.20 g, crude), which was directly used in the next step without further purification: LCMS (ESI) calculated for C₇H₆Cl₂O₂ [M−H]⁻: 191, 193 (3:2), found 191, 193 (3:2).

Step b:

To a stirred solution of 4, 5-dichloro-2-(hydroxymethyl)phenol (0.20 g, 1.04 mmol) in DCM (10 mL) was added PBr₃ (0.56 g, 2.07 mmol) dropwise at room temperature under nitrogen atmosphere. The reaction solution was stirred at room temperature for 30 min under nitrogen atmosphere. The resulting solution was quenched with water (20 mL) and extracted with EA (3×20 mL). The combined organic layers were washed with brine (3×20 mL), dried over anhydrous Na₂SO₄ and filtered. The filtrate was concentrated under reduced pressure to afford 2-(bromomethyl)-4,5-dichlorophenol as a dark grey oil (0.20 g, crude), which was directly used in the next step without further purification: LCMS (ESI) calculated for C₇H₅BrCl₂₀ [M−H]: 253, 255, 257 (2:3:1), found 253, 255, 257 (2:3:1).

Step c:

To a mixture of 2-(bromomethyl)-4,5-dichlorophenol (0.20 g, 0.78 mmol) and K₂CO₃ (0.22 g, 1.56 mmol) in ACN (10 mL) was added (2-methylpiperidin-4-yl)methanol (0.15 g, 1.17 mmol) at room temperature. The reaction mixture was allowed to warm to 40° C. and stirred for 1 h. After cooling to room temperature, the resulting mixture was filtered. The filtrate was concentrated under reduced pressure. The residue was purified by Prep-HPLC with the following conditions: Column: X Bridge C₁₈ OBD Prep Column 100 Å, 10 μm, 19 mm×250 mm; Mobile Phase A: water with 10 mmol/L NH₄HCO₃, Mobile Phase B: ACN; Flow rate: 20 mL/min; Gradient: 43% B to 65% B in 9 min; Detector: UV 254/210 nm; Retention time: Rt₁: 8.10 min, Rt₂: 8.60 min.

The faster-eluting isomer was obtained as Compound 29 (4,5-dichloro-2-(((2S,4S)-rel-4-(hydroxymethyl)-2-methylpiperidin-1-yl)methyl)phenol) as a light yellow solid (90 mg, 37%): LCMS (ESI) calculated for C₁₄H₁₉Cl₂NO₂ [M+H]⁺: 304, 306 (3:2), found 304, 306 (3:2); ¹H NMR (300 MHz, CD₃OD) δ 7.12 (s, 1H), 6.79 (s, 1H), 4.33 (d, J=14.7 Hz, 1H), 3.46-3.29 (m, 3H), 3.05-2.84 (m, 1H), 2.55-2.30 (m, 1H), 2.17 (td, J=12.4, 2.6 Hz, 1H), 1.86-1.49 (m, 3H), 1.22 (d, J=6.2 Hz, 3H), 1.33-0.97 (m, 2H).

The slower-eluting isomer was obtained as Compound 24 (4,5-dichloro-2-(((2R,4S)-rel-4-(hydroxymethyl)-2-methylpiperidin-1-yl)methyl)phenol) as a light yellow solid (6.5 mg, 3%): LCMS (ESI) calculated for C₁₄H₁₉Cl₂NO₂ [M+H]⁺: 304, 306 (3:2), found 304, 306 (3:2); ¹H NMR (300 MHz, CD₃OD) δ 7.15 (s, 1H), 6.79 (s, 1H), 3.84 (d, J=2.0 Hz, 2H), 3.38 (d, J=6.2 Hz, 2H), 3.27-3.13 (m, 1H), 2.79-2.59 (m, 2H), 1.92-1.61 (m, 3H), 1.50 (m, 1H), 1.38-1.18 (m, 1H), 1.13 (d, J=6.7 Hz, 3H).

### Example 24. Compound 46 (4,5-dichloro-2-[1-[4-(hydroxymethyl)piperidin-1-yl]ethyl]phenol)

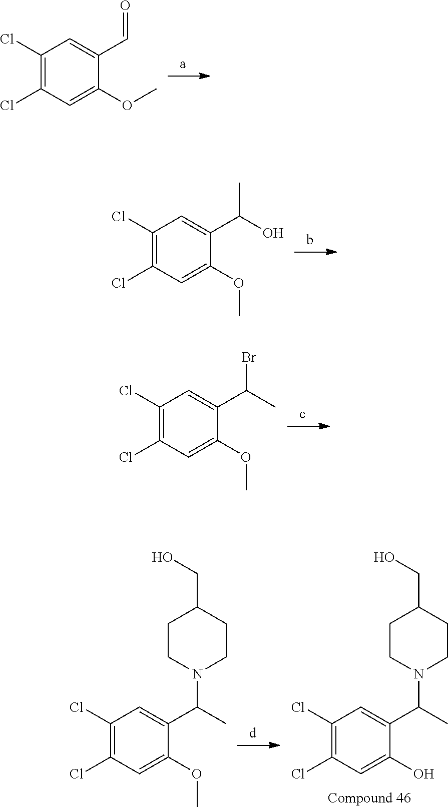

Step a:

To a stirred solution of 4,5-dichloro-2-methoxybenzaldehyde (1.50 g, 7.32 mmol) in THE (50 mL) was added MeMgBr (9 mL, 9.00 mmol, 1 M in THF) at 0° C. under nitrogen atmosphere. The reaction solution was allowed to warm to room temperature and stirred for 1 h under nitrogen atmosphere. The resulting solution was quenched with water (50 mL) and extracted with EA (3×50 mL). The combined organic layers were washed with brine (2×30 mL) and dried over anhydrous Na₂SO₄. After filtration, the filtrate was concentrated under reduced pressure. The residue was purified by silica gel column chromatography, eluted with PE/EA (5/1) to afford 1-(4,5-dichloro-2-methoxyphenyl)ethan-1-ol as an off-white solid (1.40 g, 87%): ¹H NMR (300 MHz, CDCl₃) δ 7.43 (d, J=0.7 Hz, 1H), 6.91 (s, 1H), 5.03 (q, J=6.3 Hz, 1H), 3.82 (s, 3H), 1.43 (d, J=6.5 Hz, 3H).

Step b:

To a stirred solution of 1-(4,5-dichloro-2-methoxyphenyl)ethan-1-ol (0.50 g, 2.26 mmol) in DCM (10 mL) was added PBr₃ (1.22 g, 4.52 mmol) dropwise at room temperature. After stirring for 15 min at room temperature, the resulting solution was quenched with water (10 mL) and extracted with EA (3×40 mL). The combined organic layers were washed with brine (2×20 mL) and dried over anhydrous Na₂SO₄. After filtration, the filtrate was concentrated under reduced pressure to afford 1-(1-bromoethyl)-4,5-dichloro-2-methoxybenzene as a light yellow oil (0.50 g, crude), which was used in next step directly without further purification: ¹H NMR (400 MHz, CDCl₃) δ 7.57 (s, 1H), 6.96 (s, 1H), 5.55 (q, J=7.0 Hz, 1H), 3.90 (s, 3H), 2.01 (d, J=7.0 Hz, 3H).

Step c:

To a stirred mixture of 1-(1-bromoethyl)-4,5-dichloro-2-methoxybenzene (0.12 g, 1.06 mmol) and K₂CO₃ (0.19 g, 1.41 mmol) in ACN (10 mL) were added piperidin-4-ylmethanol (0.12 g, 1.06 mmol) at room temperature. The reaction mixture was allowed to warm to 40° C. and stirred for 2 h. The resulting mixture was diluted with water (50 mL) and extracted with EA (3×50 mL). The combined organic layer was washed with brine (2×30 mL) and dried over anhydrous Na₂SO₄. After the filtration, the filtrate was concentrated under reduced pressure. The residue was purified by Prep-HPLC with the following conditions: Column: X Bridge C₁₈ OBD Prep Column 100 Å, 10 μm, 19 mm×250 mm; Mobile Phase A: water with 10 mmol/L NH₄HCO₃, Mobile Phase B: ACN; Flow rate: 20 mL/min; Gradient: 40% B to 80% B in 8 min; Detector: UV 210 nm; Retention time: 7.57 min. The fractions containing desired product were collected and concentrated under reduced pressure to afford [1-[1-(4,5-dichloro-2-methoxyphenyl)ethyl]piperidin-4-yl]methanol as an off-white solid (0.10 g, 43%): LCMS (ESI) calculated for C₁₅H₂₁Cl₂NO₂ [M+H]⁺: 318, 320 (3:2), found 318, 320 (3:2); ¹H NMR (400 MHz, CDCl₃) δ 7.53 (s, 1H), 6.94 (s, 1H), 3.90-3.78 (m, 1H), 3.82 (s, 3H), 3.51 (d, J=6.3 Hz, 2H), 3.18 (d, J=11.1 Hz, 1H), 2.82 (d, J=11.4 Hz, 1H), 1.99 (t, J=10.3 Hz, 1H), 1.89-1.73 (m, 2H), 1.65 (d, J=13.1 Hz, 1H), 1.48-1.40 (m, 1H), 1.37-1.10 (m, 5H).

Step d:

To a stirred solution of [1-[1-(4,5-dichloro-2-methoxyphenyl)ethyl]piperidin-4-yl]methanol (0.70 g, 2.20 mmol) in DCM (20 mL) was added BBr₃ (1.65 g, 6.60 mmol) at room temperature. After stirring for 2 h at room temperature, the resulting mixture was quenched with ice water (10 mL), and then was neutralized with saturated aq. NaHCO₃ to pH 7˜8. The resulting solution was concentrated under reduced pressure. The residue was purified by Prep-HPLC with the following conditions: Column: X Bridge C₁₈ OBD Prep Column 100 Å, 10 μm, 19 mm×250 mm; Mobile Phase A: water with 10 mmol/L NH₄HCO₃, Mobile Phase B: ACN; Flow rate: 20 mL/min; Gradient: 42% B to 50% B in 12 min; Detector: UV 210 nm; Retention time: 8.60 min. The fractions containing desired product were collected and concentrated under reduced pressure to afford Compound 46 (4,5-dichloro-2-[1-[4-(hydroxymethyl)piperidin-1-yl]ethyl]phenol) as an off-white solid (250 mg, 37%): LCMS (ESI) calculated for C₁₄H₁₉Cl₂NO₂ [M+H]⁺: 304, 306 (3:2), found 304, 306 (3:2); ¹H NMR (300 MHz, CDCl₃) δ 7.07 (s, 1H), 6.93 (s, 1H), 3.84 (s, 1H), 3.52 (d, J=6.3 Hz, 2H), 3.05 (d, J=11.5 Hz, 2H), 2.38 (t, J=11.6 Hz, 1H), 2.18 (t, J=11.6 Hz, 1H), 1.85 (d, J=13.2 Hz, 2H), 1.47-1.23 (m, 6H).

### Example 25. Compound 54 (1-[(4,5-dichloro-2-hydroxyphenyl)methyl]-4-(hydroxymethyl)piperidine-4-carbonitrile)

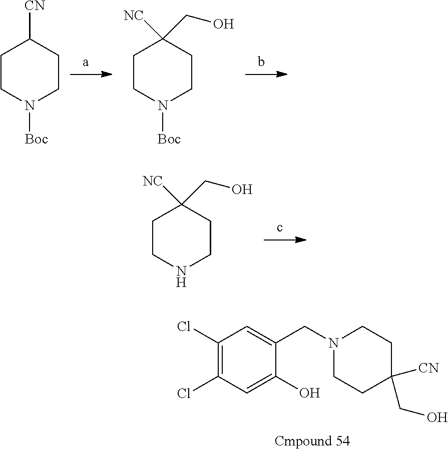

Step a:

To a stirred solution of tert-butyl 4-cyanopiperidine-1-carboxylate (1.00 g, 4.76 mmol) in THE (8 mL) was added LDA (2.85 mL, 5.71 mmol, 2 M in THF) dropwise at −78° C. under argon atmosphere. The reaction mixture was stirred at −78° C. for 1 h. Then paraformaldehyde (0.17 g, 5.71 mmol) was added to the solution. The resulting mixture was allowed to warm to room temperature and stirred for 1 h under argon atmosphere. The resulting solution was quenched with saturated aq. NH₄Cl (2 mL) at −78° C. and diluted with water (50 mL). The aqueous layer was extracted with EA (3×30 mL). The combined organic layers were washed with brine (3×30 mL) and dried over anhydrous Na₂SO₄ and filtered. The filtrate was concentrated under reduced pressure. The residue was purified by silica gel column chromatography, eluted with PE/EA (3/1) to afford tert-butyl 4-cyano-4-(hydroxymethyl)piperidine-1-carboxylate as an off-white semisolid (0.60 g, 42%): LCMS (ESI) calculated for C₁₂H₂₀N₂O₃ [M+H]⁺: 241, found 241; ¹H NMR (300 MHz, CDCl₃) δ 4.37-4.01 (m, 2H), 3.64 (s, 2H), 3.14-2.91 (m, 2H), 1.99-1.83 (m, 2H), 1.51-1.28 (m, 11H).

Step b:

To a stirred solution of tert-butyl 4-cyano-4-(hydroxymethyl)piperidine-1-carboxylate (0.20 g, 0.83 mmol) in DCM (2 mL) was added TFA (2 mL) at room temperature. After stirring for 1 h at room temperature, the resulting solution was concentrated under reduced pressure. The residue was dissolved in water (10 mL), and adjusted pH value with saturated aq. K₂CO₃ to 8. The aqueous layer was extracted with DCM (10×20 mL). The combined organic layers were dried over anhydrous K₂CO₃ and filtered. The filtrate was concentrated under reduced pressure to afford 4-(hydroxymethyl)piperidine-4-carbonitrile as a yellow oil (0.10 g, crude), which was used in next step directly without further purification.

Step c:

To a stirred solution of Intermediate 1 (0.10 g, 0.52 mmol) in DCE (3 mL) were added 4-(hydroxymethyl)piperidine-4-carbonitrile (73 mg, 0.52 mmol), HOAc (31 mg, 0.52 mmol) and NaBH(OAc)₃ (0.33 g, 1.57 mmol) at room temperature. After stirring for 3 h at room temperature, the resulting mixture was quenched with water (1 mL) and concentrated under reduced pressure. The residue was purified by Prep-HPLC with following conditions: Column: XBridge Shield RP18 OBD Column 19×250 mm, 10 μm; Mobile Phase A: water with 10 mmoL/L NH₄HCO₃, Mobile Phase B: ACN; Flow rate: 20 mL/min; Gradient: 40% B to 78% B in 9 min; Detector: UV 210 nm; Retention time: 8.23 min. The fractions containing desired product were collected and concentrated under reduced pressure to afford Compound 54 (1-[(4,5-dichloro-2-hydroxyphenyl)methyl]-4-(hydroxymethyl)piperidine-4-carbonitrile) as an off-white solid (24 mg, 14%): LCMS (ESI) calculated for C₁₄H₁₆Cl₂N₂O₂ [M+H]⁺ 315, 317 (3:2), found 315, 317 (3:2); ¹H NMR (300 MHz, DMSO-d₆) δ 10.7 (br, 1H), 7.33 (s, 1H), 6.94 (s, 1H), 5.39 (s, 1H), 3.54 (s, 2H), 3.43 (s, 2H), 2.93-2.76 (m, 2H), 2.28-2.06 (m, 2H), 1.90-1.69 (m, 2H), 1.62-1.39 (m, 2H).

### Example 26. Compound 60 (4,5-dibromo-2-((4-(hydroxymethyl)piperidin-1-yl)methyl)phenol)

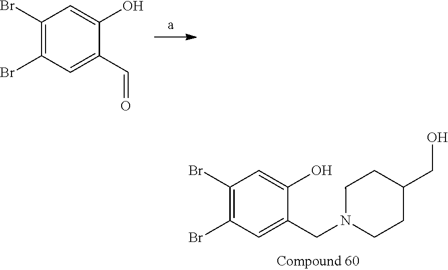 [SMILES: O=Cc1cc(Br)c(Br)cc1O ; OCC1CCN(Cc2cc(Br)c(Br)cc2O)CC1]

Step a:

To a Biotage 20 mL vial equipped with a magnetic stir bar was added 4-piperidinemethanol (53.9 uL, 300 umol) to a solution of 4,5-dibromo-2-hydroxybenzaldehyde (80.0 mg, 286 umol) in anhydrous THE (2 mL). The solution was stirred at room temperature for 3 hour. The solution was cooled to 0° C. and AcOH (20 mL, 372 umol) was added dropwise to the reaction followed by portionwise addition of NaBH(OAc)₃ (78.4 mg, 372 umol). The reaction was stirred from 0° C. to room temperature overnight. The reaction was quenched by addition of NaOH 1N dropwise at 0° C. (5 mL), while being transferred in an Erlenmeyer, and it was further stirred for 30 minutes. The reaction was then diluted with DCM (40 mL) and sat. NaHCO₃ solution (20 mL) is added to the biphasic mixture. It was then transferred to an extraction funnel. Layers were separated and the aqueous layer was extracted with DCM (3×20 mL). The organic layers were then washed with brine (2×30 mL), dried over Na₂SO₄, filtered and evaporated to dryness. The resulting crude solid was then purified by flash chromatography using 30-100% EA in hexanes. The resulting white solid was then partially dissolved in a mixture of ACN/water (40:60) and lyophilized to afford Compound 60 (4,5-dibromo-2-((4-(hydroxymethyl)piperidin-1-yl)methyl)phenol) (61.4 mg, 48%) as a white solid. LCMS (ESI) calculated for C₁₃H₁₇Br₂NO₂ [M]⁺: 377.0/379.0 (1:2), found [M+H]⁺: 378.0/380.0 (1:2). ¹H NMR (500 MHz, DMSO) δ 7.44 (s, 1H), 7.07 (s, 1H), 3.59 (s, 2H), 3.25 (d, J=6.3 Hz, 2H), 2.86 (d, J=11.7 Hz, 2H), 2.04 (td, J=11.8, 2.3 Hz, 2H), 1.68 (dd, J=12.7, 1.5 Hz, 2H), 1.46-1.34 (m, 1H), 1.14 (qd, J=12.5, 3.8 Hz, 2H).

### Example 27. Compound 63 ((1-(4,5-dibromo-2-hydroxybenzyl)piperidin-4-yl)(pyrrolidin-1-yl)methanone)

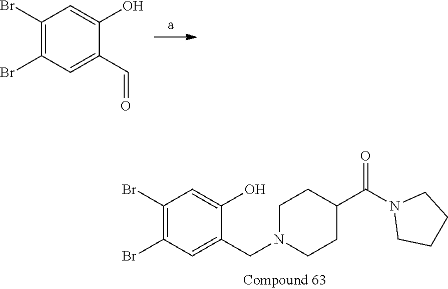 [SMILES: O=Cc1cc(Br)c(Br)cc1O ; O=C(C1CCN(Cc2cc(Br)c(Br)cc2O)CC1)N1CCCC1]

Step a:

To a Biotage 20 mL vial equipped with a magnetic stirred bar was added 4-piperidinyl(1-pyrrolidinyl)methanone hydrochloride (656 mg, 3.0 mmol), Et₃N (0.42 mL, 3.0 mmol), and dibromosalisaldehyde (663 mg, 3.3 mmol). The reagents were dissolved in anhydrous THE (10 mL) and the solution was stirred at room temperature for 4 hours. The solution was cooled to 0° C. and AcOH (0.35 mL, 6.0 mmol) was added dropwise. Then, NaBH(OAc)₃ (1.27 g, 6.0 mmol) was added portion wise and the reaction was stirred from 0° C. to room temperature overnight. The reaction was quenched by addition of HCl 0.5 N at 0° C. (10 mL) and stirred for another 30 minutes. The reaction was then diluted with DCM (40 mL) and sat. NaHCO₃ solution (30 mL) is added to the biphasic mixture. The biphasic mixture was transferred to an extraction funnel. Layers were separated and the aqueous layer was extracted with DCM (3×20 mL). The organic layers were combined and washed with brine (2×30 mL), dried over Na₂SO₄, filtered and evaporated to dryness. The resulting gum was then purified by flash chromatography using a gradient of 60% EA in hexanes to 10% MeOH/EA. The product was re-purified by reverse phase (C-18 column) using a gradient of 5-100% ACN/H₂O. The desired fraction were combined and lyophilized to afford Compound 63 (1-(4,5-dibromo-2-hydroxybenzyl)piperidin-4-yl)(pyrrolidin-1-yl)methanone) (35.2 mg, 6.6%) as a white solid. LCMS (ESI) calculated for C₁₇H₂₂Br₂N₂O₂ [M+H]⁺: 444.0/446.0 (1:2), found 444.8/446.8, 351 (1:2)¹H NMR (400 MHz, cdcl₃) δ 7.17 (s, 1H), 7.10 (s, 1H), 3.63 (d, J=9.1 Hz, 2H), 3.46 (t, J=6.8 Hz, 4H), 3.04 (d, J=11.8 Hz, 2H), 2.41 (t, J=10.9 Hz, 1H), 2.15 (s, 2H), 2.02-1.91 (m, 3H), 1.91-1.81 (m, 3H), 1.81-1.73 (m, 2H).

### Example 28. Compound 65 (4,5-dichloro-2-((4-(hydroxymethyl)piperidin-1-yl)methyl)phenol)

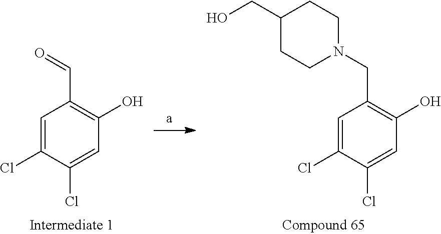 [SMILES: O=Cc1cc(Cl)c(Cl)cc1O ; OCC1CCN(Cc2cc(Cl)c(Cl)cc2O)CC1]

Step a:

To a solution of piperidin-4-ylmethanol (63 mg, 0.55 mmol), Intermediate 1 (0.10 g, 0.53 mmol), acetic acid (30 mg, 0.50 mmol) in DCE (3 mL) was added NaBH(OAc)₃ (0.32 g, 1.51 mmol) at room temperature under nitrogen atmosphere. After stirring for 3 h at room temperature under nitrogen atmosphere, the reaction mixture was quenched with water (20 mL) and extracted with DCM (3×30 mL). The combined organic layers were washed with brine (2×30 mL), dried over anhydrous Na₂SO₄ and filtered. The filtrate was concentrated under reduced pressure. The residue was purified by Prep-HPLC with the following conditions: Column: XBridge Prep C₁₈ OBD Column 190 mm×150 mm, 5 μm; Mobile Phase A: water with 10 mmoL/L NH₄HCO₃, Mobile Phase B: ACN; Flow rate: 20 mL/min; Gradient: 40% B to 55% B in 7 min; Detector: UV 254/220 nm; Retention time: 6.33 min. The fractions containing desired product were collected and concentrated under reduced pressure to afford Compound 65 (4,5-dichloro-2-((4-(hydroxymethyl)piperidin-1-yl)methyl)phenol) as an off-white solid (34 mg, 22%): LCMS (ESI) calculated for C₁₃H₁₇Cl₂NO₂ [M+H]⁺: 290, 292 (3:2), found 290, 292 (3:2); ¹H NMR (400 MHz, DMSOd₆+D₂O) δ 7.32 (s, 1H), 6.93 (s, 1H), 3.61 (s, 2H), 3.25 (d, J=6.4 Hz, 2H), 2.84 (d, J=11.2 Hz, 2H), 2.04 (t, J=9.6 Hz, 2H), 1.69 (d, J=11.2 Hz, 2H), 1.40-1.36 (m, 1H), 1.17 (q, J=8.0 Hz, 2H).

The Compounds in Table 1a below were prepared in an analogous fashion to that described for Compound 65, starting from 4,5-dichloro-2-hydroxy-benzaldehyde and the corresponding amine, which were prepared as described herein, or which were available from commercial sources.

TABLE 1a

| Compound | | | |
|---|---|---|---|
| Number | Structure | Chemical Name | MS: (M + H)⁺ & ¹H NMR |
| 20 | 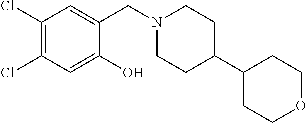 [SMILES: Oc1cc(Cl)c(Cl)cc1CN1CCC(C2CCOCC2)CC1] | 4,5-dichloro-2-[[4-(oxan- 4-yl)piperidin-1- yl]methyl]phenol | [M + H]⁺: 344, 346 (3:2); ¹H NMR (300 MHz, CD₃OD) δ 7.17 (s, 1H), 6.85 (s, 1H), 3.95 (dd, J = 12.5, 3.2 Hz, 2H), 3.70 (s, 2H), 3.35 (d, J = 18.1 Hz, 2H), 3.02 (d, J = 11.8 Hz, 2H), 2.16 (td, J = 12.2, 2.3 |
| | | | Hz, 2H), 1.89-1.77 (m, 2H), |
| | | | 1.66 (d, J = 10.9 Hz, 2H), |
| | | | 1.41-1.09 (m, 6H). |
| 22 | 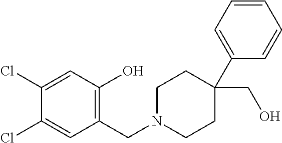 [SMILES: OCC1(c2ccccc2)CCN(Cc2cc(Cl)c(Cl)cc2O)CC1] | 4,5-dichloro-2-[[4- (hydroxymethyl)-4- phenylpiperidin-1- yl]methyl]phenol | [M + H]⁺: 366, 368 (3:2); ¹H NMR (300 MHz, DMSO-d₆) δ 7.38-7.22 (m, 5H), 7.16 (t, J = 6.7 Hz, 1H), 6.90 (s, 1H), 3.50 (s, 2H), 3.29 (s, 2H), 2.69-2.58 (m, 2H), 2.24-2.02 (m, 4H), 1.86 (t, J = 11.9 Hz, 2H). |
| 25 | 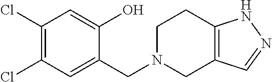 [SMILES: Oc1cc(Cl)c(Cl)cc1CN1CCc2[nH]ncc2C1] | 4,5-dichloro-2-((6,7- dihydro-1H-pyrazolo[4,3- c]pyridin-5(4H)- yl)methyl)phenol | [M + H]⁺: 298, 300 (3:2); ¹H NMR (400 MHz, DMSO-d₆) δ 12.00 (br, 1H), 7.41 (s, 1H), 7.34 (s, 1H), 6.99 (s, 1H), 3.78 (s, 2H), 3.51 (s, 2H), 2.80 (t, J = 5.8 |
| | | | Hz, 2H), 2.69 (t, J = 5.9 Hz, |
| | | | 2H). |
| 26 | 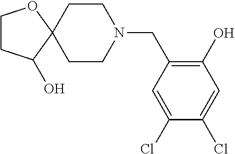 [SMILES: Oc1cc(Cl)c(Cl)cc1CN1CCC2(CC1)OCCC2O] | 8-(4,5-dichloro-2- hydroxybenzyl)-1-oxa-8- azaspiro[4.5]decan-4-ol | [M + H]⁺: 332, 334 (3:2); ¹H NMR (400 MHz, CD₃OD) δ 7.23 (s, 1H), 6.89 (s, 1H), 4.01-3.90 (m, 2H), 3.90-3.79 (m, 1H), 3.77 (s, 2H), 2.92-2.72 (m, 2H), 2.70-2.52 (m, 2H), 2.37-2.22 (m, 1H), 1.96- 1.50 (m, 5H). |
| 27 | 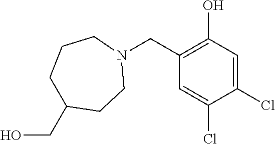 [SMILES: OCC1CCCN(Cc2cc(Cl)c(Cl)cc2O)CC1] | 4,5-dichloro-2-[[4- (hydroxymethyl)azepan-1- yl]methyl]phenol | [M + H]⁺: 304, 306 (3:2); ¹H NMR (300 MHz, DMSO-d₆) δ 7.30 (s, 1H), 6.91 (s, 1H), 3.72 (s, 2H), 3.17 (d, J = 6.4 Hz, 2H), 2.78-2.48 (m, 4H), 1.80- 1.43 (m, 5H), 1.48-1.02 (m, 2H). |
| 30 | 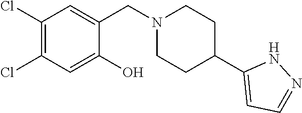 [SMILES: Oc1cc(Cl)c(Cl)cc1CN1CCC(c2ccn[nH]2)CC1] | 2-((4-(1H-pyrazol-5- yl)piperidin-1-yl)methyl)- 4,5-dichlorophenol | [M + H]⁺: 326, 328 (3:2); ¹H NMR (400 MHz, CD₃OD) δ 7.51 (s, 1H), 7.23 (s, 1H), 6.90 (s, 1H), 6.15 (s, 1H), 3.75 (s, 2H), 3.07 (d, J = 11.7 Hz, 2H), 2.85-2.73 (m, 1H), 2.40- 2.29 (m, 2H), 2.03 (d, J = |
| | | | 13.3 Hz, 2H), 1.80 (m, 2H). |
| 31 | 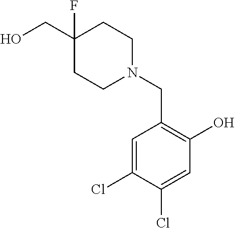 [SMILES: OCC1(F)CCN(Cc2cc(Cl)c(Cl)cc2O)CC1] | 4,5-dichloro-2-[[4-fluoro- 4-(hydroxymethyl)piperidin- l-yl]methyl]phenol | [M + H]⁺: 308, 310 (3:2); ¹H NMR (400 MHz, CD₃OD) δ 7.21 (s, 1H), 6.87 (s, 1H), 3.71 (s, 2H), 3.51 (d, J = 19.8 Hz, 2H), 2.85-2.71 (m, 2H), 2.49- 2.39 (m, 2H), 1.96-1.62 (m, 4H); ¹⁹F NMR (282 MHz, CD₃OD) δ −169.55. |
| 32 | 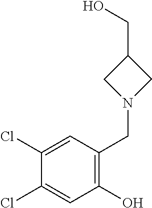 [SMILES: OCC1CN(Cc2cc(Cl)c(Cl)cc2O)C1] | 4,5-dichloro-2-[[3- (hydroxymethyl)azetidin- 1-yl]methyl]phenol | [M + H]⁺: 262, 264 (3:2); ¹H NMR (300 MHz, CDCl₃) δ 7.02 (s, 1H), 6.91 (s, 1H), 3.81-3.71 (m, 4H), 3.44 (t, J = 7.9 Hz, 2H), 3.18 (t, J = 7.0 Hz, 2H), 2.83-2.65 (m, 1H). |
| 33 | 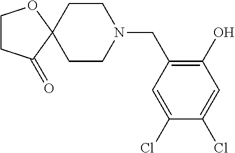 [SMILES: O=C1CCOC12CCN(Cc1cc(Cl)c(Cl)cc1O)CC2] | 8-[(4,5-dichloro-2- hydroxyphenyl)methyl]-1- oxa-8-azaspiro[4.5]decan- 4-one | [M + H]⁺: 330, 332 (3:2); ¹H NMR (300 MHz, CDCl₃) δ 7.35 (s, 1H), 6.97 (s, 1H),4.08 (t, J = 7.2 Hz, 2H), 3.59 (s, 2H), 2.74 (d, J = 11.6 Hz, 2H), 2.58 (t, J = 7.2 Hz, 2H), 2.33-2.27 (m, 2H), 1.61-1.51 (m, 4H). |
| 34 | 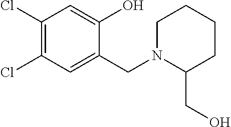 [SMILES: OCC1CCCCN1Cc1cc(Cl)c(Cl)cc1O] | 4,5-dichloro-2-[[2- (hydroxymethyl)piperidin- 1-yl]methyl]phenol | [M + H]⁺: 290, 292 (3:2); ¹H NMR (300 MHz, CD₃OD) δ 7.17 (s, 1H), 6.83 (s, 1H), 4.39 (d, J = 14.3 Hz, 1H), 3.86-3.64 (m, 2H), 3.50 (d, J = 14.4 Hz, 1H), 2.91 (dt, J = 12.7, 4.1 Hz, 1H), 2.59-2.48 (m, 1H), 2.39-2.16 (m, 1H), 1.82- |
| | | | 1.39 (m, 6H). |
| 35 | 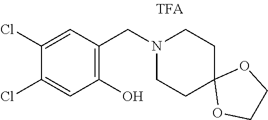 [SMILES: Oc1cc(Cl)c(Cl)cc1CN1CCC2(CC1)OCCO2] | 4,5-dichloro-2-[1,4-dioxa- 8-azaspiro[4.5]decan-8- ylmethyl]phenol trifluoroacetic acid | [M + H]⁺: 318, 320 (3:2); ¹H NMR (400 MHz, DMSO-d₆) δ 11.10 (s, 1H), 9.44 (s, 1H), 7.70 (s, 1H), 7.15 (s, 1H), 4.27 (s, 2H), 3.93 (s, 4H), 3.42 (s, 2H), 3.10 (s, 2H), 1.90 (s, 4H); ¹⁹F NMR (282 MHz, DMSO-d₆) δ −73.72. |
| 36 | 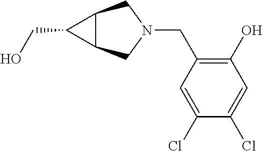 [SMILES: OC[C@@H]1[C@H]2CN(Cc3cc(Cl)c(Cl)cc3O)C[C@@H]12] | 4,5-dichloro-2- (((1R,5S,6R)-6- (hydroxymethyl)-3- azabicyclo[3.1.0]hexan-3- yl)methyl)phenol | [M + H]⁺: 288, 290 (3:2); ¹H NMR (400 MHz, DMSO-d₆) δ 7.30 (s, 1H), 6.95 (s, 1H), 3.67 (s, 2H), 3.24 (d, J = 6.6 Hz, 2H), 2.94 (d, J = 9.1 Hz, 2H), 2.45-2.38 (m, 2H), 1.33 (d, J = 2.9 Hz, 2H), 1.19-1.09 (m, 1H). |
| 37 | 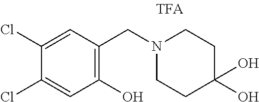 [SMILES: Oc1cc(Cl)c(Cl)cc1CN1CCC(O)(O)CC1] | 1-(4,5-dichloro-2- hydroxybenzyl)piperidine- 4,4-diol trifluoroacetic acid | [M + H]⁺: 292, 294 (3:2); ¹H NMR (400 MHz, DMSO-d₆) δ 11.10 (s, 1H), 10.25 (s, 1H), 7.70 (s, 1H), 7.15 (s, 1H), 4.31 (s, 2H), 3.52 (s, 4H), 2.59 (s, 4H); ¹⁹F NMR (282 MHz, DMSO-d₆) δ −74.08. |
| 38 | 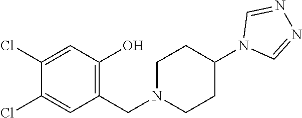 [SMILES: Oc1cc(Cl)c(Cl)cc1CN1CCC(n2cnnc2)CC1] | 4,5-dichloro-2-[[4-(4H- 1,2,4-triazol-4-yl)piperidin- 1-yl]methyl]phenol | [M + H]⁺: 327, 329 (3:2); ¹H NMR (400 MHz, DMSO-d₆) δ 8.64 (s, 2H), 7.40 (s, 1H), 7.00 (s, 1H), 4.28-4.15 (m, 1H), 3.60 (s, 2H), 2.98-2.89 (m, 2H), 2.25-2.15 (m, 2H), 2.06- 1.87 (m, 4H). |
| 39 | 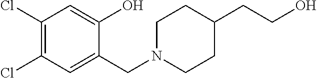 [SMILES: OCCC1CCN(Cc2cc(Cl)c(Cl)cc2O)CC1] | 4,5-dichloro-2-((4-(2- hydroxyethyl)piperidin-1- yl)methyl)phenol | [M + H]⁺: 304, 306 (3:2); ¹H NMR (400 MHz, DMSO-d₆) δ 7.33 (s, 1H), 6.94 (s, 1H), 3.62 (s, 2H), 3.44 (t, J = 6.4 Hz, 2H), 2.85 (dd, J = 11.8, 3.4 Hz, |
| | | | 2H), 2.05 (td, J = 11.8, 2.5 |
| | | | Hz, 2H), 1.72-1.63 (m, 2H), |
| | | | 1.50-1.32 (m, 3H), 1.14 (m, |
| | | | 2H). |
| 40 | 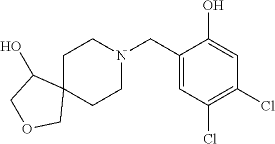 [SMILES: Oc1cc(Cl)c(Cl)cc1CN1CCC2(CC1)COCC2O] | 8-(4,5-dichloro-2- hydroxyphenyl)-2-oxa-8- azaspiro[4.5]decan-4-ol | [M + H]⁺: 332, 334 (3:2); ¹H NMR (300 MHz, CD₃OD) δ 7.19 (s, 1H), 6.87 (s, 1H), 4.08 (dd, J = 9.6, 4.9 Hz, 1H), 3.98-3.94 (m, 1H), 3.75-3.59 (m, 5H), 2.71 (d, J = 9.5 Hz, 2H), 2.50-2.39 (m, 2H), 1.95- 1.81 (m, 1H), 1.66-1.54 (m, 3H). |
| 41 | 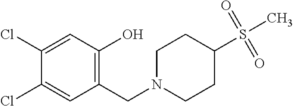 [SMILES: CS(=O)(=O)C1CCN(Cc2cc(Cl)c(Cl)cc2O)CC1] | 4,5-dichloro-2-((4- (methylsulfonyl)piperidin- 1-yl)methyl)phenol | [M + H]⁺: 338, 340 (3:2); ¹H NMR (300 MHz, DMSO-d₆) δ 7.35 (s, 1H), 6.96 (s, 1H), 3.57 (s, 2H), 3.16-2.87 (m, 6H), 2.16- 1.94 (m, 4H), 1.70-1.50 (m, 2H). |
| 42 | 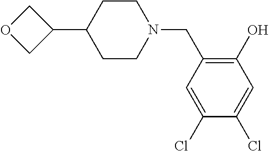 [SMILES: Oc1cc(Cl)c(Cl)cc1CN1CCC(C2COC2)CC1] | 4,5-dichloro-2-[[4-(oxetan- 3-yl)piperidin-1- yl]methyl]phenol | [M + H]⁺: 316, 318 (3:2); ¹H NMR (300 MHz, DMSO-d₆) δ 7.31 (s, 1H), 6.91 (s, 1H), 4.60-4.49 (m, 2H), 4.38-4.30 (m, 2H), 3.60 (s, 2H), 2.89-2.80 (m, 2H), 2.78-2.68 (m, 1H), 2.12-1.97 (m, 2H), 1.67- 1.52 (m, 3H), 1.11-0.92 (m, 2H). |
| 43 | 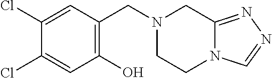 [SMILES: Oc1cc(Cl)c(Cl)cc1CN1CCn2cnnc2C1] | 4,5-dichloro-2- [5H,6H,7H,8H- [1,2,4]triazolo[4,3- a]pyrazin-7- ylmethyl]phenol | [M + H]⁺: 299, 301 (3:2), found 299, 301 (3:2); ¹H NMR (400 MHz, DMSO- d₆) δ 10.35 (s, 1H), 8.43 (s, 1H), 7.47 (s, 1H), 7.03 (s, 1H), 4.05 (m, 2H), 3.77 (s, |
| | | | 2H), 3.72 (s, 2H),2.88 (m, |
| | | | 2H). |
| 44 | 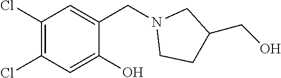 [SMILES: OCC1CCN(Cc2cc(Cl)c(Cl)cc2O)C1] | 4,5-dichloro-2-[[3- (hydroxymethyl)pyrrolidin- 1-yl]methyl]phenol | [M + H]⁺: 276, 278 (3:2); 1H NMR (400 MHz, CD₃OD) δ 7.22 (s, 1H), 6.87 (s, 1H), 3.83 (s, 2H), 3.58-3.40 (m, 2H), 2.87- 2.65 (m, 3H), 2.59 (dd, J = |
| | | | 10.0, 5.8 Hz, 1H), 2.50-2.38 |
| | | | (m, 1H), 2.11-1.97 (m, 1H), |
| | | | 1.68-1.55 (m, 1H). |
| 45 | 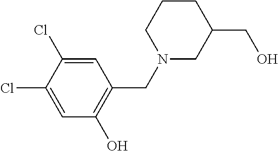 [SMILES: OCC1CCCN(Cc2cc(Cl)c(Cl)cc2O)C1] | 4,5-dichloro-2-[[3- (hydroxymethyl)piperidin- 1-yl]methyl]phenol | [M + H]⁺: 290, 292 (3:2); ¹H NMR (400 MHz, DMSO-d₆) δ 7.34 (s, 1H), 6.95 (s, 1H), 3.70-3.55 (m, 2H), 3.31 (dd, J = 10.6, 5.2 Hz, 1H),3.19(dd, J = 10.6, 5.2 Hz, 1H), 2.94-2.86 (m, 1H), 2.79 (d, J = 11.2 Hz, 1H), 2.04 (m, 1H), 1.80 (m, 1H), 1.65 (m, 3H), 1.46 (m, |
| | | | 1H), 1.03-0.84 (m, 1H). |
| 47 | 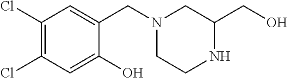 [SMILES: OCC1CN(Cc2cc(Cl)c(Cl)cc2O)CCN1] | 4,5-dichloro-2-((3- (hydroxymethyl)piperazin- 1-yl)methyl)phenol | [M + H]⁺: 291, 293 (3:2); ¹H NMR (400 MHz, DMSO-d₆) δ 7.36 (s, 1H), 6.97 (s, 1H), 4.66-4.53 (m, 1H), 3.67-3.53 (m, 2H), 3.38-3.27 (m, 3H), 2.90 (d, |
| | | | J = 12.1 Hz, 1H), 2.83-2.78 |
| | | | (m, 1H), 2.74-2.63 (m, 2H), |
| | | | 2.10-2.03 (m, 1H), 1.82- |
| | | | 1.74 (m, 1H). |
| 48 | 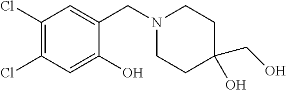 [SMILES: OCC1(O)CCN(Cc2cc(Cl)c(Cl)cc2O)CC1] | 1-[(4,5-dichloro-2- hydroxyphenyl)methyl]-4- (hydroxymethyl)piperidin- 4-ol | [M + H]⁺: 306, 308 (3:2); ¹H NMR (300 MHz, CD₃OD) δ 7.19 (s, 1H), 6.85 (s, 1H), 3.74 (s, 2H), 3.37 (s, 2H), 2.76 (m, J = 11.9, 3.8 Hz, 2H), 2.58 (d, J = 11.7, 3.2 Hz, 2H), 1.81- 1.54 (m, 4H). |
| 49 | 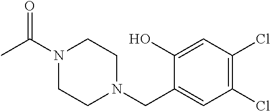 [SMILES: CC(=O)N1CCN(Cc2cc(Cl)c(Cl)cc2O)CC1] | 1-[4-[(4,5-dichloro-2- hydroxyphenyl)methyl] piperazin-1-yl]ethan-1-one | [M + H]⁺: 303, 305 (3:2); ¹H NMR (300 MHz, DMSO-d₆) δ 10.61 (s, 1H), 7.36 (s, 1H), 6.96 (s, 1H), 3.52 (s, 2H), 3.47-3.36 (m, 4H), 2.47-2.31 (m, 4H), 1.96 (s, 3H). |
| 50 | 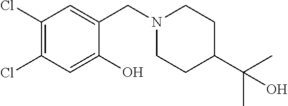 [SMILES: CC(C)(O)C1CCN(Cc2cc(Cl)c(Cl)cc2O)CC1] | 4,5-dichloro-2-[[4-(2- hydroxypropan-2- yl)piperidin-1- yl]methyl]phenol | [M + H]⁺: 318, 320 (3:2); ¹H NMR (300 MHz, DMSO-d₆) δ 7.32 (s, 1H), 6.94 (s, 1H), 3.60 (s, 2H), 2.97 (d, J = 11.6 Hz, 2H), 2.00 (t, J = 9.6 Hz, 2H), 1.72 (d, J = 9.9 Hz, 2H), 1.35-1.12 (m, 3H), 1.05 (s, |
| | | | 6H). |
| 51 | 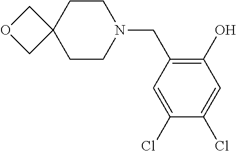 [SMILES: Oc1cc(Cl)c(Cl)cc1CN1CCC2(CC1)COC2] | 4,5-dichloro-2-[2-oxa-7- azaspiro [3.5] nonan-7- ylmethyl] phenol | [M + H]⁺: 302, 304 (3:2); ¹H NMR (400 MHz, DMSO-d₆) δ 7.34 (s, 1H), 6.96 (s, 1H), 4.27 (s, 4H), 3.53 (s, 2H), 2.38-2.32 (m, 4H), 1.80 (m, 4H). |
| 52 | 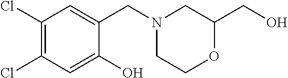 [SMILES: OCC1CN(Cc2cc(Cl)c(Cl)cc2O)CCO1] | 4,5-dichloro-2-[[2- (hydroxymethyl)morpholin- 4-yl]methyl]phenol | [M + H]⁺: 292, 294 (3:2); ¹H NMR (300 MHz, DMSO-d₆) δ 10.80 (br, 1H), 7.35 (s, 1H), 6.96 (s, 1H), 4.63 (s, 1H), 3.77 (dd, J = 11.4, 1.5 Hz, 1H), 3.62-3.19 |
| | | | (m, 6H), 2.78 (d, J = 11.4 |
| | | | Hz, 1H), 2.65 (d, J = 11.4 |
| | | | Hz, 1H), 2.10 (td, J = 11.5, |
| | | | 3.3 Hz, 1H), 1.91-1.80 (t, J = |
| | | | 11.5 Hz, 1H). |
| 53 | 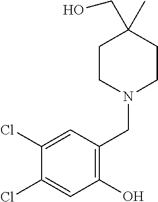 [SMILES: CC1(CO)CCN(Cc2cc(Cl)c(Cl)cc2O)CC1] | 4,5-dichloro-2-[[4- (hydroxymethyl)-4- methylpiperidin-1-yl] methyl] phenol | [M + H]⁺: 304, 306 (3:2); ¹H NMR (400 MHz, DMSO-d₆) δ 7.32 (s, 1H), 6.94 (s, 1H), 3.64 (s, 2H), 3.16 (s, 2H), 2.57-2.48 (m, 2H), 2.40-2.29 (m, 2H), 1.53-1.40 (m, 2H), 1.28- 1.18 (m,2H), 0.86 (s, 3H). |
| 55 | 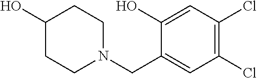 [SMILES: Oc1cc(Cl)c(Cl)cc1CN1CCC(O)CC1] | 1-[(4,5-dichloro-2- hydroxyphenyl)methyl] piperidin-4-ol | [M + H]⁺: 276, 278 (3:2); ¹H NMR (300 MHz, DMSO-d₆) δ 7.30 (s, 1H), 6.92 (s, 1H), 3.57 (s, 2H), 3.53-3.45 (m, 1H), 2.75- 2.62 (m, 2H), 2.17 (t, J = |
| | | | 10.5 Hz, 2H), 1.83-1.68 (m, |
| | | | 2H), 1.47-1.29 (m, 2H). |
| 56 | 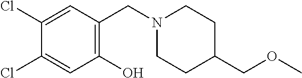 [SMILES: COCC1CCN(Cc2cc(Cl)c(Cl)cc2O)CC1] | 4,5-dichloro-2-[[4- (methoxymethyl)piperidin- 1-yl]methyl]phenol | [M + H]⁺: 304, 306 (3:2); ¹H NMR (300 MHz, DMSO-d₆) δ 7.30 (s, 1H), 6.91 (s, 1H), 3.58 (s, 2H), 3.21 (s, 3H), 3.17 (d, J = 17.4 Hz, 2H), 2.83 (d, J = |
| | | | 11.5 Hz, 2H), 2.02 (t, J = |
| | | | 11.2 Hz, 2H), 1.69-1.58 (m, |
| | | | 3H), 1.25-1.10 (m, 2H). |
| 57 | 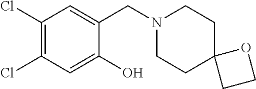 [SMILES: Oc1cc(Cl)c(Cl)cc1CN1CCC2(CCO2)CC1] | 4,5-dichloro-2-[1-oxa-7- azaspiro[3.5]nonan-7- ylmethyl]phenol | [M + H]⁺: 302, 304 (3:2); ¹H NMR (300 MHz, CD₃OD) δ 7.20 (s, 1H), 6.87 (s, 1H), 4.53 (t, J = 7.9 Hz, 2H), 3.66 (s, 2H), 2.55- 2.37 (m, 6H), 2.01-1.91 (m, 4H). |
| 58 | 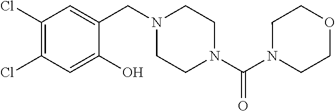 [SMILES: O=C(N1CCOCC1)N1CCN(Cc2cc(Cl)c(Cl)cc2O)CC1] | (4-(4,5-dichloro-2- hydroxybenzyl)piperazin- 1-yl)(morpholino) methanone | [M]⁺: 374.0. ¹H NMR (400 MHz, CDCl₃): δ 7.04 (s, 1H), 6.93 (s, 1H), 3.68- 3.66 (m, 7H), 3.35-3.25 (m, 8H), 2.56 (m, 4H). |
| 59 | 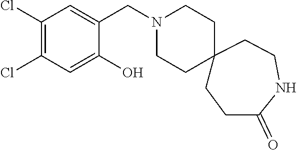 [SMILES: O=C1CCC2(CCN1)CCN(Cc1cc(Cl)c(Cl)cc1O)CC2] | 3-(4,5-dichloro-2- hydroxybenzyl)-3,9- diazaspiro[5.6]dodecan- 10-one | [M + H]⁺: 357.2, 359.2. ¹H- NMR (400 MHz, CDCl₃) δ 7.01 (s, 1H), 6.90 (s, 1H), 5.95 (br s, 1H), 3.66 (s, 2H), 3.18 (dd, J = 10.2, 5.8 Hz, 2H), 2.85-2.24 (m, 6H), 2.02-1.34 (m, 8H). |
| 61 | 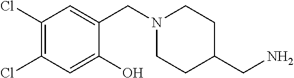 [SMILES: NCC1CCN(Cc2cc(Cl)c(Cl)cc2O)CC1] | 2-((4- (aminomethyl)piperidin-1- yl)methyl)-4,5- dichlorophenol | [M + H]⁺: 289.2. ¹H-NMR (500 MHz, DMSO-d6) δ 11.17 (s, 1H), 9.42 (s, 1H), 7.85 (s, 3H), 7.66 (s, 1H), 7.15 (s, 1H), 4.18 (s, 2H), 3.16 (s, 1H), 2.99 (s, 2H), |
| | | | 2.72 (s,2H), 1.90 (d, J = |
| | | | 13.0 Hz, 2H), 1.81 (s, 2H), |
| | | | 1.43-1.30 (m, 2H). |
| 62 | 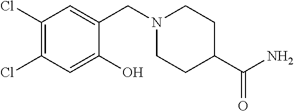 [SMILES: NC(=O)C1CCN(Cc2cc(Cl)c(Cl)cc2O)CC1] | l-(4,5-dichloro-2- hydroxybenzyl) piperidine- 4-carboxamide | [M + H]⁺: 303.1. ¹H-NMR (500 MHz, DMSO-d6) δ 7.25 (s, 1H), 6.86 (s, 1H), 3.54 (s, 2H), 3.18 (d, J = 6.3 Hz, 2H), 2.79 (d, J = 11.7 Hz, 2H), 1.97 (dt, J = 2.4, 11.8 Hz, 2H), 1.60 (dd, J = 1.7, 12.8 Hz, 2H), 1.37- |
| | | | 1.26 (m, 1H), 1.07 (dq, J = |
| | | | 3.8, 12.3 Hz, 2H) |
| 64 | 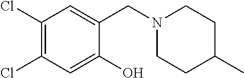 [SMILES: CC1CCN(Cc2cc(Cl)c(Cl)cc2O)CC1] | 4,5-dichloro-2-((4- methylpiperidin-1- yl)methyl)phenol | [M + H]⁺: 274.0 ¹H NMR (500 MHz, DMSO) δ 8.32 (s, 1H), 7.33 (s, 1H), 6.94 (s, 1H), 3.61 (s, 2H), 2.83 (d, J = 11.7 Hz, 2H), 2.09- 2.01 (m, 2H), 1.63 (d, J = |
| | | | 12.1 Hz, 2H), 1.47-1.30 |
| | | | (m, 1H), 1.13 (qd, J = 12.6, |
| | | | 3.7 Hz, 2H), 0.90 (d, J = 6.5 |
| | | | Hz, 3H). |
| 66 | 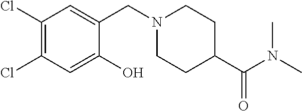 [SMILES: CN(C)C(=O)C1CCN(Cc2cc(Cl)c(Cl)cc2O)CC1] | 1-(4,5-dichloro-2- hydroxybenzyl)-N,N- dimethylpiperidine-4- carboxamide | [M + H]⁺: 331.1. ¹H NMR (400 MHz, DMSO) δ 7.34 (s, 1H), 6.95 (s, 1H), 3.60 (s, 2H), 3.00 (s, 3H), 2.87 (d, J = 11.7 Hz, 2H), 2.80 (s, 3H), 2.65-2.60 (m, 1H), 2.12 (dd, J = 11.9, 9.4 Hz, 1H), 1.64 (d, J = 11.0 Hz, |
| | | | 1H), 1.59-1.51 (m, 2H) |

### Example 29. Evaluation of Kv1.3 Potassium Channel Blocker Activities

This assay is used to evaluate the disclosed compounds' activities as Kv1.3 potassium channel blockers.

### Cell Culture

CHO-K1 cells stably expressing Kv1.3 were grown in DMEM containing 10% heat-inactivated FBS, 1 mM Sodium Pyruvate, 2 mM L-Glutamine and G418 (500 μg/ml). Cells were grown in culture flasks at 37° C. in a 500 CO₂-humidified incubator.

### Solutions

The cells were bathed in an extracellular solution containing 140 mM NaCl, 4 mM KCl, 2 mM CaCl₂, 1 mM MgCl₂, 5 mM Glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The currents were evoked by applying 100 ms depolarizing pulses from −90 mV (holding potential) to +40 mV were applied with 0.1 Hz frequency. Control (compound-free) and compound pulse trains for each compound concentration applied contained 20 pulses. 10 second breaks were used between pulse trains (see Table A below).

### Patch Clamp Recordings and Compound Application

Whole cell current recordings and compound application were enabled by means of an automated patch clamp platform Patchliner (Nanion Technologies GmbH). EPC 10 patch clamp amplifier (HEKA Elektronik Dr. Schulze GmbH) along with Patchmaster software (HEKA Elektronik Dr. Schulze GmbH) was used for data acquisition. Data were sampled at 10 kHz without filtering. Passive leak currents were subtracted online using a P/4 procedure (HEKA Elektronik Dr. Schulze GmbH). Increasing compound concentrations were applied consecutively to the same cell without washouts in between. Total compound incubation time before the next pulse train was not longer than 10 seconds. Peak current inhibition was observed during compound equilibration.

### Data Analysis

AUC and peak values were obtained with Patchmaster (HEKA Elektronik Dr. Schulze GmbH). To determine IC₅₀, the last single pulse in the pulse train corresponding to a given compound concentration was used. Obtained AUC and peak values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ value is the concentration at which current inhibition is half-maximal, [compound] is the applied compound concentration, A is the fraction of current that is not blocked and nH is the Hill coefficient.

### Example 30. Evaluation of hERG Activities

This assay is used to evaluate the disclosed compounds' inhibition activities against the hERG channel.

hERG Electrophysiology

This assay is used to evaluate the disclosed compounds' inhibition activities against the hERG channel.

### Cell Culture

CHO-K1 cells stably expressing hERG were grown in Ham's F-12 Medium with Glutamine containing 10% heat-inactivated FBS, 1% Penicillin/Streptomycin, Hygromycin (100 μg/ml) and G418 (100 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM Glucose, 10 mM HEPES; pH adjusted to 7.4 with NaOH; 295-305 mOsm. The internal solution contained 50 mM KCl, 10 mM NaCl, 60 mM KF, 20 mM EGTA, 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The voltage protocol (see Table B) was designed to simulate voltage changes during a cardiac action potential with a 300 ms depolarization to +20 mV (analogous to the plateau phase of the cardiac action potential), a repolarization for 300 ms to −50 mV (inducing a tail current) and a final step to the holding potential of −80 mV. The pulse frequency was 0.3 Hz. Control (compound-free) and compound pulse trains for each compound concentration applied contained 70 pulses.

### Patch Clamp Recordings and Compound Application

Whole cell current recordings and compound application were enabled by means of an automated patch clamp platform Patchliner (Nanion). EPC 10 patch clamp amplifier (HEKA) along with Patchmaster software (HEKA Elektronik Dr. Schulze GmbH) was used for data acquisition. Data were sampled at 10 kHz without filtering. Increasing compound concentrations were applied consecutively to the same cell without washouts in between.

### Data Analysis

AUC and PEAK values were obtained with Patchmaster (HEKA Elektronik Dr. Schulze GmbH). To determine IC₅₀ the last single pulse in the pulse train corresponding to a given compound concentration was used. Obtained AUC and PEAK values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ is the concentration at which current inhibition is half-maximal, [compound] is the applied compound concentration, A is the fraction of current that is not blocked and nH is the Hill coefficient.

Table 1 provides a summary of the inhibition activities of certain selected compounds against Kv1.3 potassium channel and hERG channel.

TABLE 1
IC₅₀ (μM) values of certain exemplified compounds against
Kv1.3 potassium channel and hERG channel

| | | Kvl.3 | hERG |
|---|---|---|---|
| Compound Number | Structure | IC₅₀ | IC₅₀ |
| 1 | 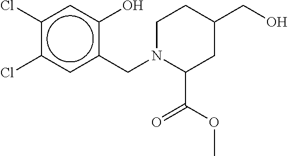 | <10 | * |
| 2 | 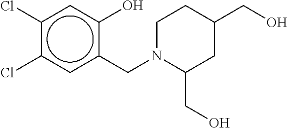 | <10 | * |
| 3 | 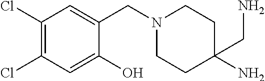 [SMILES: NCC1(N)CCN(Cc2cc(Cl)c(Cl)cc2O)CC1] | <1 | <30 |
| 4 | 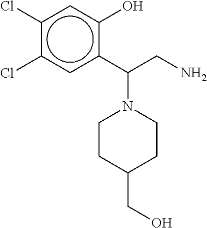 | <10 | * |
| 5 | 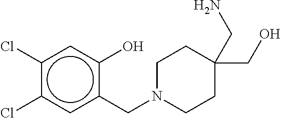 | <1 | <30 |
| 6 | 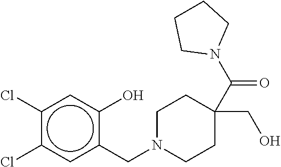 | <10 | * |
| 7 | 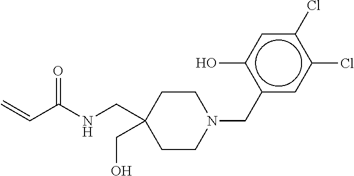 | <1 | * |
| 8 | 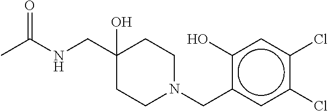 | <10 | * |
| 9 | 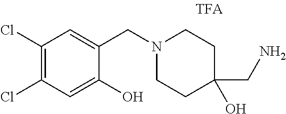 [SMILES: NCC1(O)CCN(Cc2cc(Cl)c(Cl)cc2O)CC1] | <1 | <30 |
| 10 | 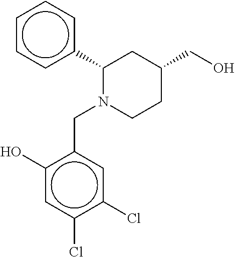 | <10 | * |
| 11 | 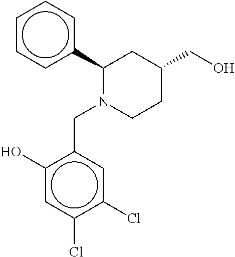 | <10 | * |
| 12 | 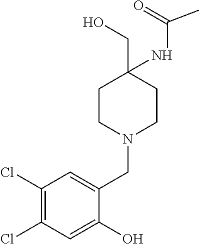 [SMILES: CC(=O)NC1(CO)CCN(Cc2cc(Cl)c(Cl)cc2O)CC1] | <1 | >30 |
| 13 | 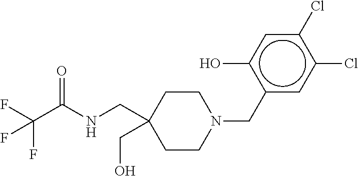 | <1 | * |
| 14 | 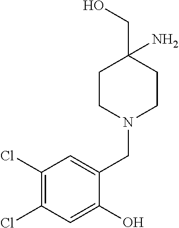 [SMILES: NC1(CO)CCN(Cc2cc(Cl)c(Cl)cc2O)CC1] | <1 | <30 |
| 15 | 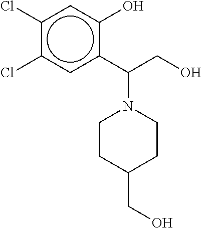 | <10 | * |
| 16 | 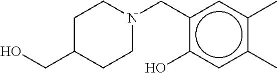 | <10 | * |
| 17 | 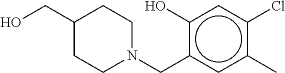 | <10 | * |
| 18 | 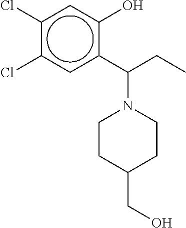 | <10 | <30 |
| 19 | 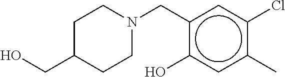 | <1 | >30 |
| 20 | 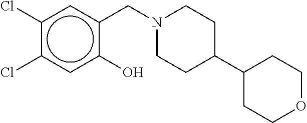 | <1 | <30 |
| 21 | 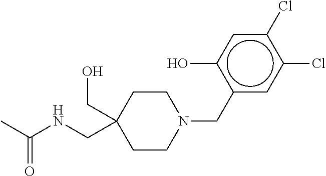 | <1 | <30 |
| 22 | 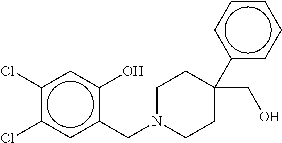 | <1 | <30 |
| 23 | 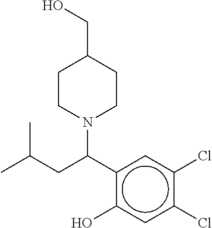 | <10 | * |
| 24 | 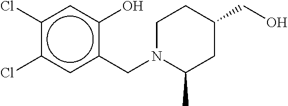 | <10 | * |
| 25 | 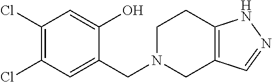 [SMILES: Oc1cc(Cl)c(Cl)cc1CN1CCc2[nH]ncc2C1] | <10 | <30 |
| 26 | 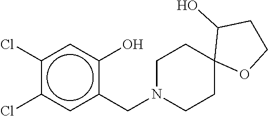 | <1 | <30 |
| 27 | 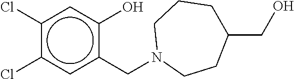 | <1 | <30 |
| 28 | 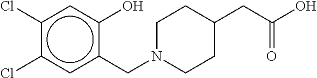 | <10 | * |
| 29 | 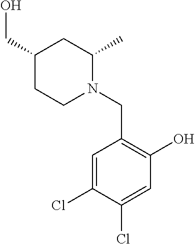 [SMILES: C[C@H]1C[C@@H](CO)CCN1Cc1cc(Cl)c(Cl)cc1O] | <1 | <30 |
| 30 | 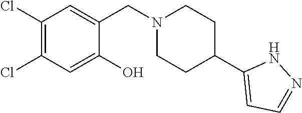 [SMILES: Oc1cc(Cl)c(Cl)cc1CN1CCC(c2ccn[nH]2)CC1] | <10 | <30 |
| 31 | 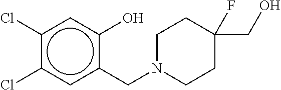 | <10 | * |
| 32 | 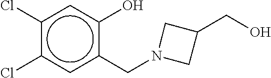 | <10 | <30 |
| 33 | 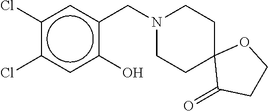 | <10 | * |
| 34 | 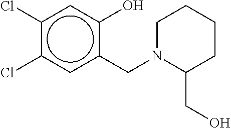 | <10 | * |
| 35 | 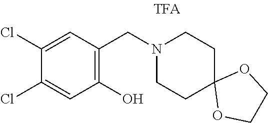 [SMILES: Oc1cc(Cl)c(Cl)cc1CN1CCC2(CC1)OCCO2] | <10 | * |
| 36 | 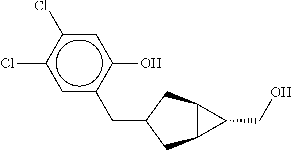 | <10 | * |
| 37 | 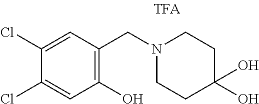 [SMILES: Oc1cc(Cl)c(Cl)cc1CN1CCC(O)(O)CC1] | <10 | * |
| 38 | 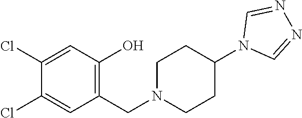 [SMILES: Oc1cc(Cl)c(Cl)cc1CN1CCC(n2cnnc2)CC1] | <10 | * |
| 39 | 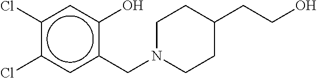 | <10 | <30 |
| 40 | 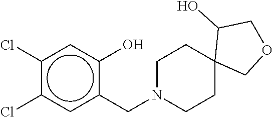 | <1 | <30 |
| 41 | 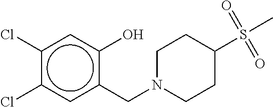 | <10 | * |
| 42 | 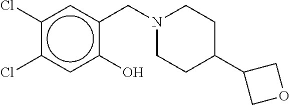 | <10 | <30 |
| 43 | 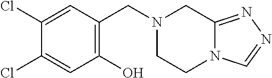 [SMILES: Oc1cc(Cl)c(Cl)cc1CN1CCn2cnnc2C1] | <10 | * |
| 44 | 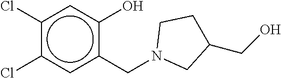 | <10 | <30 |
| 45 | 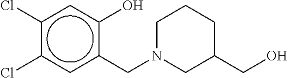 | <10 | <30 |
| 46 | 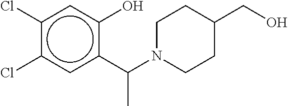 | <10 | * |
| 47 | 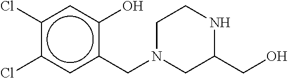 | <10 | * |
| 48 | 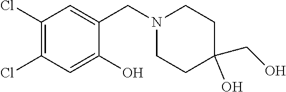 [SMILES: OCC1(O)CCN(Cc2cc(Cl)c(Cl)cc2O)CC1] | <10 | <30 |
| 49 | 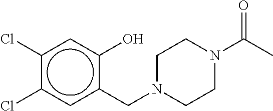 | <10 | <30 |
| 50 | 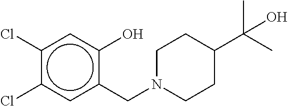 | <10 | <30 |
| 51 | 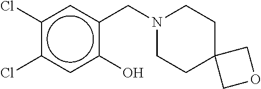 | <10 | <30 |
| 52 | 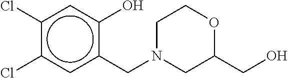 | <10 | 30 |
| 53 | 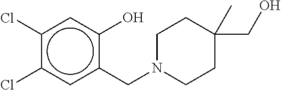 | <10 | <30 |
| 54 | 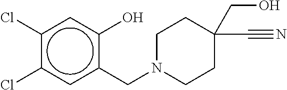 | <10 | <30 |
| 55 | 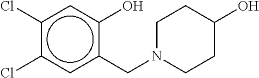 | <10 | <30 |
| 56 | 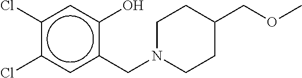 | <10 | <30 |
| 57 | 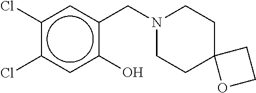 | <10 | <30 |
| 58 | 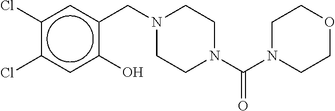 | <10 | <30 |
| 59 | 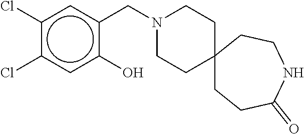 | <10 | * |
| 60 | 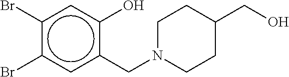 | <1 | <30 |
| 61 | 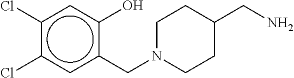 | <1 | <30 |
| 62 | 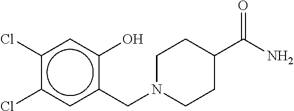 | <10 | <30 |
| 63 | 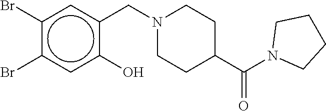 | <1 | <30 |
| 64 | 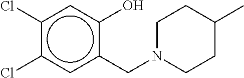 | <10 | <30 |
| 65 | 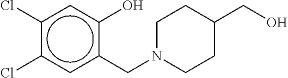 | <10 | <30 |
| 66 | 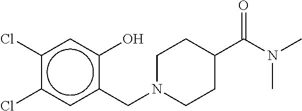 | <10 | <30 |

*Not Tested.

1. A compound of Formula I or a pharmaceutically acceptable salt thereof, 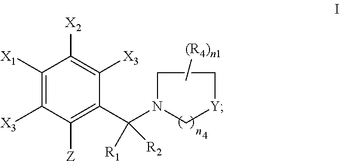
wherein
each occurrence of Y is independently C(R₄)₂, NR₄, O, S, SO, SO₂, or SO(═NRₐ);
Z is ORₐ;
X₁ is H, halogen, CN, alkyl, halogenated alkyl, cycloalkyl, or halogenated cycloalkyl;
X₂ is H, halogen, CN, alkyl, halogenated alkyl, cycloalkyl, or halogenated cycloalkyl;
each occurrence of X₃ is independently H, halogen, CN, alkyl, halogenated alkyl, cycloalkyl, or halogenated cycloalkyl;
R₁ and R₂ are each independently H, alkyl, (CR₆R₇)ₙ₃ORₐ, (CR₆R₇)ₙ₃NRₐR_{b}, (CR₆R₇)ₙ₃(C═O)NR_{b}Rₐ, or (CR₆R₇)ₙ₃NR_{b}(C═O)Rₐ;
each occurrence of R₄ is independently H, halogen, alkyl, cycloalkyl, halogenated alkyl, halogenated cycloalkyl, optionally substituted saturated heterocycle, optionally substituted aryl, optionally substituted heteroaryl, CN, oxo, (C═O)R_{b}, (C═O)OR_{b}, (CR₆R₇)ₙ₃ORₐ, (CR₆R₇)ₙ₃NRₐR_{b}, (CR₆R₇)ₙ₃SO₂Rₐ, (CR₆R₇)ₙ₃SO₂NRₐR_{b}, (CR₆R₇)ₙ₃NRₐS₂R_{b}, (CR₆R₇)ₙ₃NRₐ(C═O)R_{b}, (CR₆R₇)ₙ₃(C═O)NRₐR_{b}, (CR₆R₇)ₙ₃NRₐ(C═O)NRₐR_{b}, (C═O)(CR₆R₇)ₙ₃OR_{b}, (C═O)(CR₆R₇)ₙ₃NRₐR_{b}, or an optionally substituted 5- or 6-membered heterocycle containing 1-3 heteroatoms each selected from the group consisting of N, O, and S;
or two R₄ taken together forming an optionally substituted carbocycle, saturated heterocycle, or heteroaryl containing 0-3 heteroatoms each selected from the group consisting of N, O, and S;
each occurrence of R₆ and R₇ are independently H, alkyl, cycloalkyl, optionally substituted aryl, or optionally substituted heteroaryl;
each occurrence of Rₐ and R_{b} are independently H, alkyl, alkenyl, cycloalkyl, halogenated alkyl, halogenated cycloalkyl, optionally substituted saturated heterocycle, optionally substituted aryl, or optionally substituted heteroaryl; or alternatively Rₐ and R_{b} together with the nitrogen atom that they are connected to form an optionally substituted heterocycle comprising the nitrogen atom and 0-3 additional heteroatoms each selected from the group consisting of N, O, and S;
the alkyl, cycloalkyl, carbocycle, heterocycle, aryl, and heteroaryl in X₁, X₂, X₃, R₁, R₂, R₄, R₆, and R₇, where applicable, are optionally substituted by 1-4 substituents each independently selected from the group consisting of alkyl, cycloalkyl, halogenated cycloalkyl, halogenated alkyl, halogen, (CRₐR_{b})ₙ₃ORₐ, (CRₐR_{b})ₙ₃NRₐR_{b}, (CRₐR_{b})ₙ₃NRₐ(C═O)R_{b}, (CRₐR_{b})ₙ₃(C═O)NRₐR_{b}, and oxo where valence permits;
each occurrence of n₁ is independently an integer from 0-4 where valence permits;
each occurrence of n₃ is independently an integer from 0-4; and
each occurrence of n₄ is independently 0, 1, or 2.

2. The compound of claim 1, wherein the structural moiety 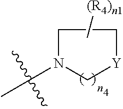
has the structure of

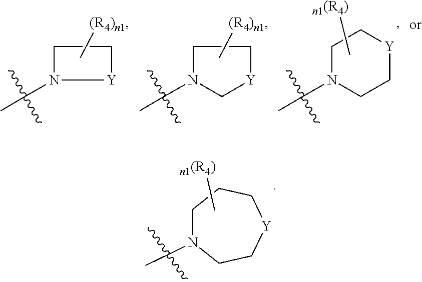

3. The compound of claim 1, wherein Y is C(R₄)₂.

4. The compound of claim 1, wherein Y is NR₄.

5. The compound of claim 1, wherein Y is O.

6. The compound of claim 1, wherein Y is S, SO, SO₂, or SO(═NRₐ).

7. The compound of claim 1, wherein Y is NR₄, CMeR₄, or CHR₄.

8. The compound of claim 1, wherein the structural moiety 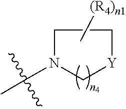
has the structure of

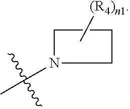

9. The compound of claim 1, wherein the structural moiety 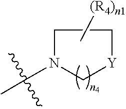
has the structure of

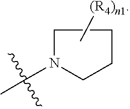

10. The compound of claim 1, wherein the structural moiety 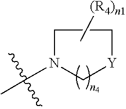
has the structure of 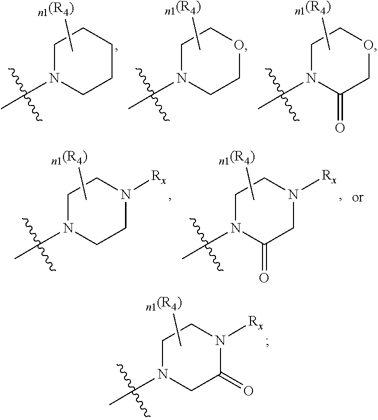
wherein Rₓ is R₄.

11. The compound of claim 1, wherein the structural moiety 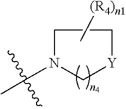
has the structure of 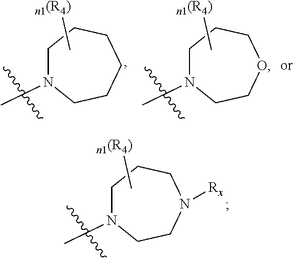
wherein Rₓ is R₄.

12. The compound of claim 1, wherein R₁ and R₂ are each independently H or alkyl.

13. The compound of claim 1, wherein R₁ and R₂ are each independently H or Me.

14. The compound of claim 1, wherein R₁ and R₂ are each independently H, (CR₆R₇)ₙ₃ORₐ, (CR₆R₇)ₙ₃NRₐR_{b}, (CR₆R₇)ₙ₃(C═O)NR_{b}Rₐ, or (CR₆R₇)ₙ₃NR_{b}(C═O)Rₐ.

15. The compound of claim 1, wherein R₁ and R₂ are each independently H, CH₂OH, CH₂NH₂, or CONH₂.

16. The compound of claim 1, wherein at least one occurrence of R₄ is independently (CR₆R₇)ₙ₃ORₐ, (CR₆R₇)ₙ₃NRₐR_{b}, (CR₆R₇)ₙ₃SO₂Rₐ, (CR₆R₇)ₙ₃NRₐ(C═O)R_{b}, or (CR₆R₇)ₙ₃(C═O)NRₐR_{b}.

17. The compound of claim 1, wherein at least one occurrence of R₄ is independently (CR₆R₇)ₙ₃NRₐ(C═O)R_{b} or (CR₆R₇)ₙ₃(C═O)NRₐR_{b}.

18. The compound of claim 1, wherein one or more occurrences of R₄ are (CR₆R₇)ₙ₃ORₐ or (CR₆R₇)ₙ₃NRₐR_{b}.

19. The compound of claim 1, wherein one or more occurrences of R₄ are ORₐ, NRₐR_{b}, —CH₂ORₐ, —CH₂NRₐR_{b}, —CH₂CH₂ORₐ, or —CH₂CH₂NRₐR_{b}.

20. The compound of claim 1, wherein at least one occurrence of R₄ is an optionally substituted 5- or 6-membered heterocycle containing 1-3 heteroatoms each selected from the group consisting of N, O, and S.

21. The compound of claim 1, wherein two R₄ taken together forming an optionally substituted carbocycle, saturated heterocycle, or heteroaryl containing 0-3 heteroatoms each selected from the group consisting of N, O, and S.

22. The compound of claim 1, wherein at least one occurrence of R₄ is CH₂OH, CH₂NH₂,

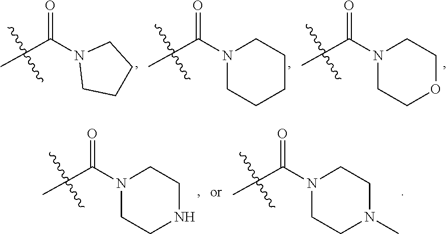

23. The compound of claim 1, wherein at least one occurrence of R₄ is a heterocycle selected from the group consisting of 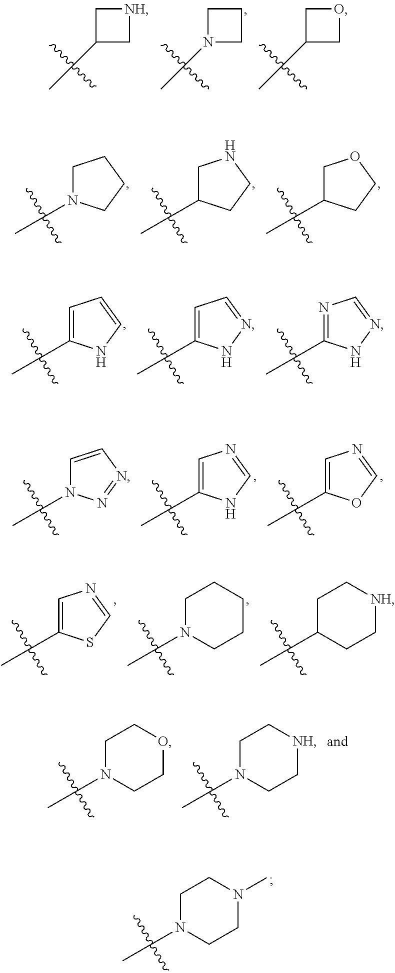
wherein the heterocycle is optionally substituted by alkyl, OH, oxo, or (C═O)C₁₋₄alkyl where valence permits.

24. The compound of claim 1, wherein at least one occurrence of R₄ is H, alkyl, cycloalkyl, optionally substituted saturated heterocycle, optionally substituted aryl, optionally substituted heteroaryl, CN, CF₃, OCF₃, ORₐ, (CR₆R₇)ₙ₃ORₐ, or oxo.

25. The compound of claim 1, wherein at least one occurrence of R₄ is (C═O)R_{b}, (C═O)OR_{b}, SO₂Rₐ, (C═O)(CR₆R₇)ₙ₃OR_{b}, (C═O)(CR₆R₇)ₙ₃NRₐR_{b}, (CR₆R₇)ₙ₃NRₐR_{b}, (CR₆R₇)ₙ₃NRₐSO₂R_{b}, (CR₆R₇)ₙ₃NRₐ(C═O)R_{b}, (CR₆R₇)ₙ₃NRₐ(C═O)NRₐR_{b}, or (CR₆R₇)ₙ₃(C═O)NRₐR_{b}.

26. The compound of claim 1, wherein at least one occurrence of R₄ is independently H or alkyl.

27. The compound of claim 1, wherein two R₄ groups taken together with the carbon atom that they are connected to form a 3-7 membered optionally substituted carbocycle or heterocycle.

28. The compound of claim 1, wherein two R₄ groups taken together with the two carbon atoms that they are connected to form a fused bicyclic system having the structure of 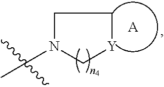
wherein A is a 3-7 membered optionally substituted carbocycle, saturated heterocycle, or heteroaryl.

29. The compound of claim 28, wherein the structural motif 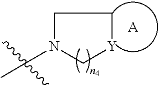
has the structure of

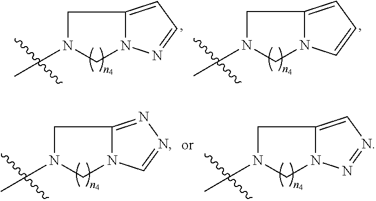

30. The compound of claim 1, wherein each occurrence of R₆ and R₇ are independently H or alkyl.

31. The compound of claim 1, wherein Z is OH or OMe.

32. The compound of claim 31, wherein Z is OH.

33. The compound of claim 1, wherein X₁ is H, CN, halogen, fluorinated alkyl, or alkyl.

34. The compound of claim 33, wherein X₁ is H, CN, Cl, Br, Me, or CF₃.

35. The compound of claim 33, wherein X₁ is H or Cl.

36. The compound of claim 1, wherein X₂ is H, CN, halogen, fluorinated alkyl, or alkyl.

37. The compound of claim 36, wherein X₂ is H, CN, Cl, Br, Me, or CF₃.

38. The compound of claim 36, wherein X₂ is H or Cl.

39. The compound of claim 1, wherein X₃ is H, halogen, CN, alkyl, or halogenated alkyl.

40. The compound of claim 39, wherein X₃ is H, Cl, Br, Me, or CF₃.

41. The compound of claim 39, wherein X₃ is H or Cl.

42. The compound of claim 1, wherein the structural moiety 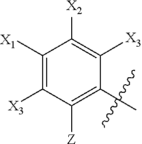
has the structure of

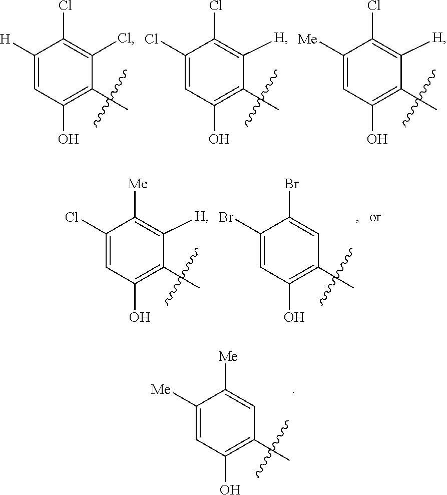

43. The compound of claim 1, wherein n₁ is 0, 1, 2, or 3.

44. The compound of claim 1, wherein each occurrence of n₃ is independently 0, 1, or 2.

45. The compound of claim 1, wherein n₄ is 1 or 2.

46. The compound of claim 1, wherein at least one occurrence of Rₐ or R_{b} is independently H, alkyl, cycloalkyl, saturated heterocycle, aryl, or heteroaryl.

47. The compound of claim 46, wherein at least one occurrence of Rₐ or R_{b} is independently H, Me, Et, Pr, or a heterocycle selected from the group consisting of 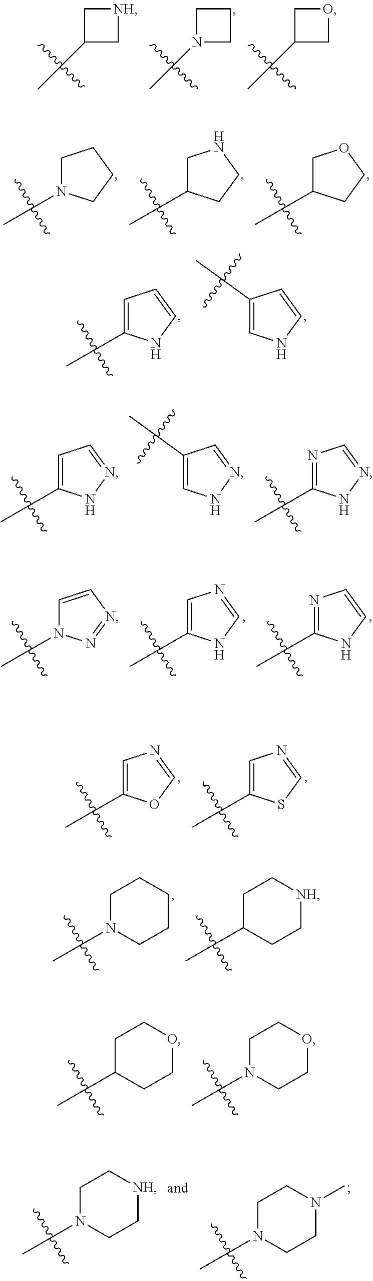
wherein the heterocycle is optionally substituted by alkyl, OH, oxo, or (C═O)C₁₋₄alkyl where valence permits.

48. The compound of claim 1, wherein Rₐ and R_{b} together with the nitrogen atom that they are connected to form an optionally substituted heterocycle comprising the nitrogen atom and 0-3 additional heteroatoms each selected from the group consisting of N, O, and S.

49. The compound of claim 1, wherein the compound is selected from the group consisting of compounds 1-66 as shown in Table 1.

50. A pharmaceutical composition comprising at least one compound according to claim 1 or a pharmaceutically acceptable salt thereof and a pharmaceutically acceptable carrier or diluent.

51. A method of treating a condition in a mammalian species in need thereof, comprising administering to the mammalian species a therapeutically effective amount of at least one compound according to claim 1 or a pharmaceutically acceptable salt thereof, wherein the condition is selected from the group consisting of cancer, an immunological disorder, a Central Nerve System (CNS) disorder, an inflammatory disorder, a gastroenterological disorder, a metabolic disorder, a cardiovascular disorder, and a kidney disease.

52. The method of claim 51, wherein the immunological disorder is transplant rejection or an autoimmune disease.

53. The method of claim 52, wherein the autoimmune disease is rheumatoid arthritis, multiple sclerosis, systemic lupus erythematosus, or Type I diabetes mellitus.

54. The method of claim 51, wherein the Central Nerve System (CNS) disorder is Alzheimer's disease.

55. The method of claim 51, wherein the inflammatory disorder is an inflammatory skin condition, arthritis, psoriasis, spondylitis, parodontitis, or an inflammatory neuropathy.

56. The method of claim 51, wherein the gastroenterological disorder is an inflammatory bowel disease.

57. The method of claim 51, wherein the metabolic disorder is obesity or Type II diabetes mellitus.

58. The method of claim 51, wherein the cardiovascular disorder is an ischemic stroke.

59. The method of claim 51, wherein the kidney disease is chronic kidney disease, nephritis, or chronic renal failure.

60. The method of claim 51, wherein the condition is selected from the group consisting of cancer, transplant rejection, rheumatoid arthritis, multiple sclerosis, systemic lupus erythematosus, Type I diabetes mellitus, Alzheimer's disease, inflammatory skin condition, inflammatory neuropathy, psoriasis, spondylitis, parodontitis, Crohn's disease, ulcerative colitis, obesity, Type II diabetes mellitus, ischemic stroke, chronic kidney disease, nephritis, chronic renal failure, and a combination thereof.

61. The method of claim 51, wherein the mammalian species is human.

62. A method of blocking Kv1.3 potassium channel in a mammalian species in need thereof, comprising administering to the mammalian species a therapeutically effective amount of at least one compound according to claim 1 or a pharmaceutically acceptable salt thereof.

63. The method of claim 62, wherein the mammalian species is human.
